(19) United States
(12) Patent Application Publication
(10) Pub. No.: US 2026/0051406 A1
(43) Pub. Date: Feb. 19, 2026

(54) PROTEIN BIOSENSOR SYSTEMS TO DETECT MUTATED COVID FROM SPUTUM AND BLOOD

(71) Applicant: Dmytro Kviatkovskyi, Bootle (GB)

(72) Inventor: Dmytro Kviatkovskyi, Bootle (GB)

(21) Appl. No.: 19/317,049

(22) Filed: Sep. 2, 2025

Related U.S. Application Data
(63) Continuation-in-part of application No. 18/423,781, filed on Jan. 26, 2024.
Application No. 18/423,781 is a continuation of application No. 17/975,105, filed on Oct. 27, 2022, now abandoned.
(60) Provisional application No. 63/294,909, filed on Dec. 30, 2021.

(51) Int. Cl. G16H 50/20; G01N 33/569; G16H 50/50

(52) U.S. Cl. CPC G16H 50/20; G01N 33/56983; G16H 50/50; G01N 2333/165; G01N 2469/20

(57) ABSTRACT

Systems for determining COVID include a protein biosensor that interacts with human biological sample and outputs the first and second signals; a wireless communication device; a memory device with detectors integrated into the memory device. The protein biosensor is configured to use two different biological components separated by an internal membrane within the protein biosensor. The concentration of the first biological component is the limit concentration for the first virus strain and the concentration of the second biological component is the limit concentration for the second virus strain. The memory device is configured to receive the first and second signals and set the first and second signal thresholds for each integrated detector. The memory device identifies the presence of the first or second virus strain in response to integrated detectors are detecting a value that is greater than or less than the first or second signal threshold.

### BACKGROUND OF THE INVENTION

### Field of the Invention

The present invention relates to diagnosing an individual's health using technical means, and more particularly, to sensor systems for detecting viral infections, e.g., COVID variants.

### Description of the Related Art

Coronavirus disease 2019 or COVID-19 (original SARS-CoV-2 virus strain) and the many thousands of SARS-CoV-2 virus strains have spread worldwide, leading to an ongoing global pandemic. The COVID pandemic has swept the entire world, becoming a fundamental global tragedy, as it is virtually impossible to isolate oneself from the virus. Viruses tend to mutate, and SARS-CoV-2 virus strain is no exception. As a consequence, severe acute respiratory syndrome coronavirus SARS-CoV-2 has many strains. Original SARS-CoV-2 virus strain is an RNA virus. Each time the virus copies itself, the RNA sequence may change, which causes mutations. The virus' traits, such as its contagiousness and lethality, also change. Currently, there are thousands of coronavirus variants.

At the end of November 2021, new dangerous strain emerged—B.1.1.529, which received the name Omicron and it has been assessed as highly dangerous by the World Health Organization. The WHO supposes that, after a few mutations, all current diagnostic means, vaccines, and coronavirus drugs may become ineffective against the Omicron strain. The Omicron strain has been found to better evade antibodies than the Delta strain, which is now the most widespread COVID strain in the world. The Omicron strain has more than 50 mutations from the original SARS-CoV-2 virus strain, and most mutations are in the gene encoding the spike protein, which is the target of most vaccines.

Over time, other very dangerous mutated COVID virus strains have sprouted up all around the world. These new variants have a different chromosomal genome structure, behave differently, and affect different vital organs in the human body, such as the Brazilian strain and Centaurus strain, which were first detected in July 2022. In August 2022, a Deltacron strain, which is a hybrid of strains Delta and Omicron, was first detected in Russia. The detection of the Pirola strain in the UK first became known on Aug. 18, 2023. The Pirola has a number of additional mutations compared to the previously identified Omicron strain, and is more contagious than its predecessors. New COVID strains continue to emerge through mutations in 2025. These variants can bind more quickly to cells, making them more transmissible. In early 2025, a new COVID strain called XEC has become the dominant global strain. Due to mutation two Omicron subvariants (KS.1.1 and KP.3.3), XEC was first detected in Germany and has since spread rapidly.

Therefore, we are facing a unique and tragic reality, in which original SARS-CoV-2 virus strain is constantly mutating, spawning new autonomous strains that may be even more hazardous for people than the original strain. While many SARS-CoV-2 virus strains have vaccines, they do not exist in quantities that might be needed and may not be located where an outbreak of a new mutated SARS-CoV-2 virus strain was to occur. Therefore, there remains a need to develop modern systems for detecting new SARS-CoV-2 virus strains with a mutated genome. Timely detection of new SARS-CoV-2 virus strains and their effective treatment is possible only if a viral disease is diagnosed comprehensively and systematically, using new methods for detecting new SARS-CoV-2 virus strains. A number of existing drawbacks of the modern medical practice can be overcome using the proposed multi-component systems of the present invention for detecting new mutated SARS-CoV-2 virus strains from human blood samples.

### SUMMARY OF THE INVENTION

The system for determining COVID disease in a person comprises a biosensor utilizing two different biological components that interacts with a blood (or sputum) sample and outputs the first and second signals; a wireless communication device configured to communicate using a wireless peer-to-peer or machine-type-communication protocol; a memory device with detectors integrated in the memory device configured to receive the first and second signals from the biosensor using one or more detectors integrated into the memory device. The blood samples include a whole blood, blood plasma, serum samples, isolated antibodies, blood compositions, blood substitutes. The sputum samples include a nasal swab, nasopharyngeal swab, oropharyngeal swab, throat swab, deep airway material, saliva. In some aspects, the biosensor includes a microfluidic chip that allows only small blood (or sputum) molecules to pass through to the biosensor and does not allow large blood (or sputum) molecules to pass through. In some aspects, the biosensor uses graphene coatings for binding to the blood (or sputum) sample.

The biosensor of present invention is configured to include the first biological component that represents proteins (or protein structures) or aptamers and the second biological component. The second biological component within the biosensor may be lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc. The first biological component and the second biological component are coupled to the same transducer within the biosensor. The first biological component and the second biological component are separated from each other by an internal membrane (e.g., a semi-permeable membrane, multilayer graphene membrane).

The concentration of the first biological component corresponds to the threshold for the first SARS-CoV-2 virus strain, and the concentration of the second biological component corresponds to the threshold for the second SARS-CoV-2 virus strain with a mutated virus genome code. The first biological component and the second biological components interact with the blood (or sputum) sample and change the chemical structure of the blood (or sputum) sample. The biosensor outputs a first signal when the first biological component interacts with the blood (or sputum) sample and changes the chemical structure of the sample, and outputs a second signal when the second biological component interacts with the blood (or sputum) sample and changes the chemical structure of the sample.

The memory device then receives the first signal and the second signal from the biosensor using one or more detectors integrated into the memory device. One or more detectors integrated into the memory device (e.g., electrochemical immunosensors, atomic magnetometers (AM), graphene-based sensors, ion drift sensors, molecular electric transducers (MET), oscillator-based sensors, flame ionization sensors, spectrometers, fluorescence microscopes, micro temperature sensors, motion sensors, conductivity sensors, electrical conductivity sensors, electrodermal activity (EDA) sensors, ECG sensors, EMG sensors, etc.) decrypt the first and second signals into values. Each detector is embedded in the memory device and coupled to the wireless communication device via the detector output. The memory device is coupled to the wireless communication device and configured to determine respective thresholds for each detector integrated into the memory device, identify that the person has the first or second SARS-CoV-2 virus strain in response to the detector are detecting a value that is greater than or less than the respective thresholds for each detector, and transmit an indication that the person has contracted the SARS-CoV-2 virus strain, via the wireless communication device, to another device.

In some aspects, the memory device controller using the controller command decoder determines a first signal threshold and a second signal threshold for each of the one or more detectors integrated into the memory device. The memory device transmits the indication to the wireless communication device via the detector output responsive to a determination that a value detected by the detector is greater than or less than the first respective threshold and responsive to a determination that a value detected by the detector is greater than or less than the second respective threshold. In some aspects, the memory device controller updates the first signal threshold and second signal threshold for each of the one or more detectors integrated into the memory device based on the detected change in the electrical, magnetic, or optical characteristics of the blood (or sputum) sample. In some aspects, the memory device controller updates the first signal threshold and second signal threshold for each of the one or more detectors integrated into the memory device based on the detected change in the temperature characteristic or characteristic of relative molecular motion of the blood (or sputum) sample.

These and other systems, methods, objects, features, and advantages of the present invention will be apparent to those skilled in the art from the following detailed description of the preferred embodiment and the drawings.

Additional features and advantages of the invention will be set forth in the description that follows, and in part will be apparent from the description, or may be learned by practice using the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed. All documents mentioned herein are hereby incorporated in their entirety by reference.

### BRIEF DESCRIPTION OF THE ATTACHED FIGURES

The accompanying drawings, which are included to provide a further understanding of the invention and are incorporated in and constitute a part of this specification, illustrate embodiments of the invention and, together with the description, serve to explain the principles of the invention.

In the drawings:

FIG. 1 illustrates components of the biosensor utilizing two different biological components for implementing the invention.

FIG. 2 is a diagram the operation of the biosensor utilizing two different biological components.

FIG. 3 illustrates an example of the composition of two different biological components within the biosensor.

FIG. 4 illustrates another example of the composition of two different biological components within the biosensor.

FIG. 5 illustrates an example of the cross-sectional of the internal membrane within the biosensor according to a first embodiment of the invention.

FIG. 6 illustrates an example of the cross-sectional of the internal membrane within the biosensor according to a second embodiment of the invention.

FIG. 7 illustrates an example of the cross-sectional of the internal membrane within the biosensor according to a third embodiment of the invention.

FIG. 8 illustrates an example of the cross-sectional of the internal membrane within the biosensor according to a fourth embodiment of the invention.

FIG. 9 is a diagram illustrating an example of the general system for detecting COVID variants of the invention.

FIG. 10 is a diagram illustrating another example of the general system for detecting COVID variants of the invention.

FIG. 11 is a diagram illustrating an example of the system with memory device sensors for implementing the invention.

FIG. 12 is a diagram illustrating another example of the system with memory device sensors for implementing the invention.

FIG. 13 is a flowchart illustrating the steps of using memory device sensors according to a first embodiment of the invention.

FIG. 14 is a flowchart illustrating the steps of using memory device sensors according to a second embodiment of the invention.

FIG. 15 is a diagram illustrating an example of the detector embedded in the internal membrane for implementing the invention.

FIG. 16 is a diagram illustrating an another example of the detector embedded in the internal membrane for implementing the invention.

FIG. 17 illustrates an example of the components of the positioner for embedding a detector into an internal membrane.

FIG. 18 illustrates another example of the components of the positioner for embedding a detector into an internal membrane.

FIG. 19 is a diagram illustrating components of the data transmission operation.

FIG. 29 illustrates components of the merging module for transmitting data.

FIG. 21 illustrates an example of the metric of differentials according to a first embodiment of the invention.

FIG. 22 illustrates examples of the metrics of differentials according to a second embodiment of the invention.

FIG. 23 illustrates examples of the metrics of differentials according to a third embodiment of the invention.

FIG. 24 illustrates examples of the metrics of differentials according to a fourth embodiment of the invention.

FIG. 25 is a diagram illustrating the medical analytics platform of the invention.

FIG. 26 is a diagram illustrating components for implementing the medical analytics platform.

FIG. 27 is a diagram illustrating the analysis of medical data of the invention.

FIG. 28 is a diagram illustrating hardware components for implementing the combinatorial data analysis.

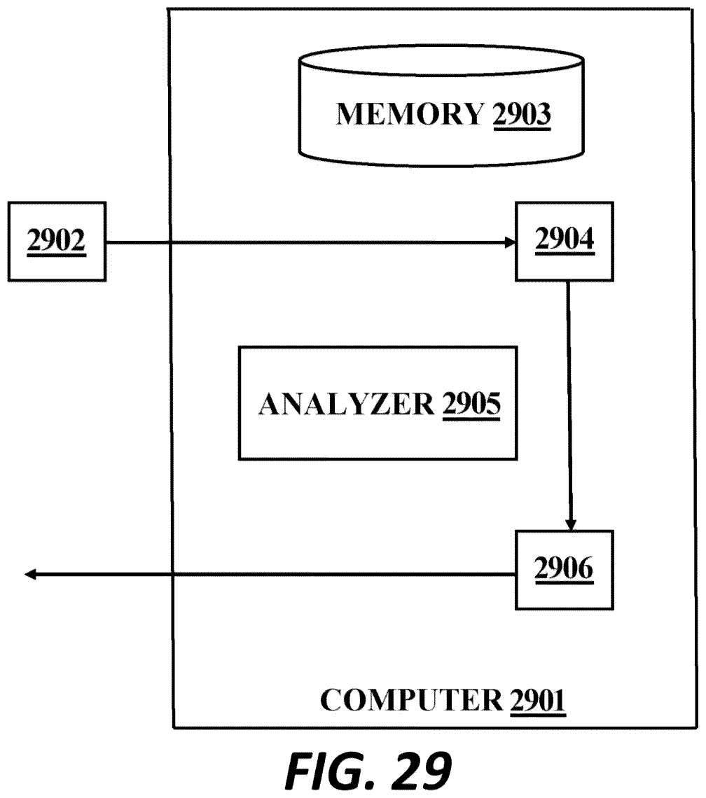

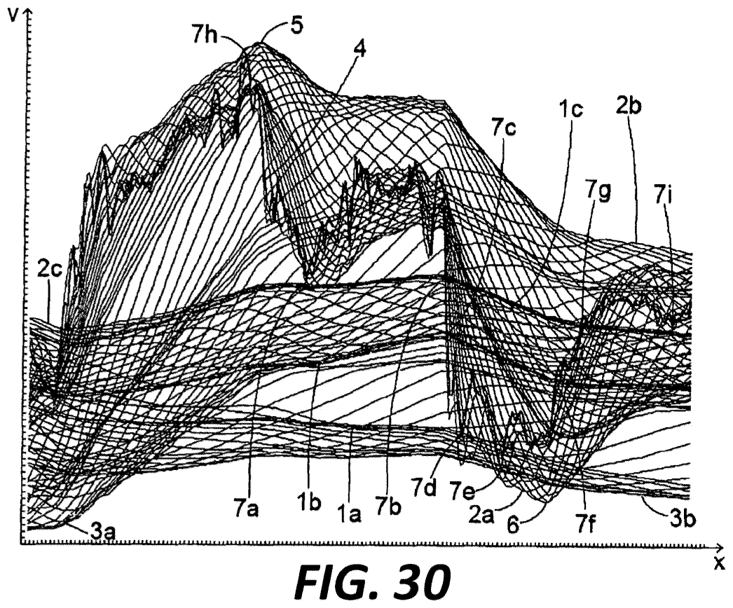

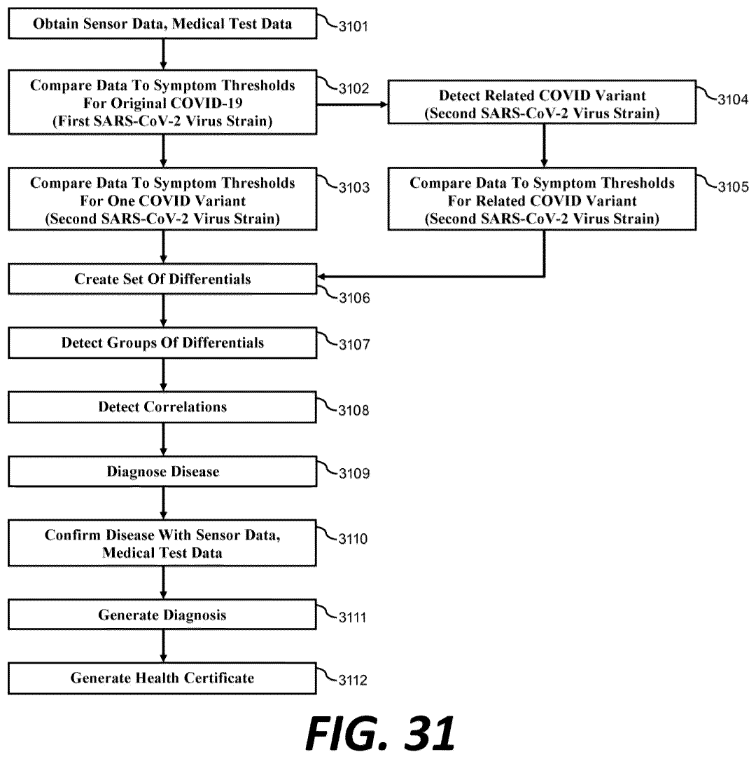

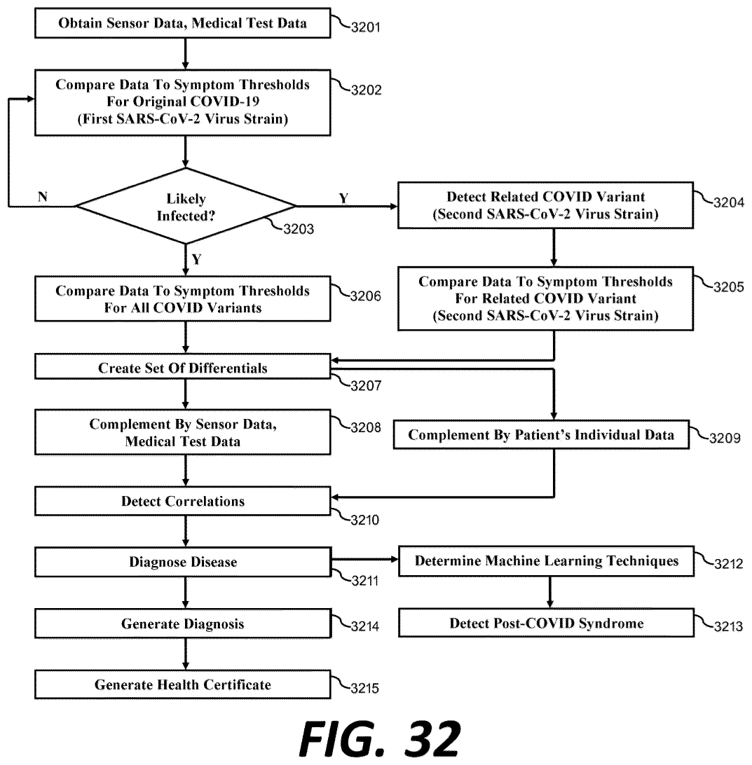

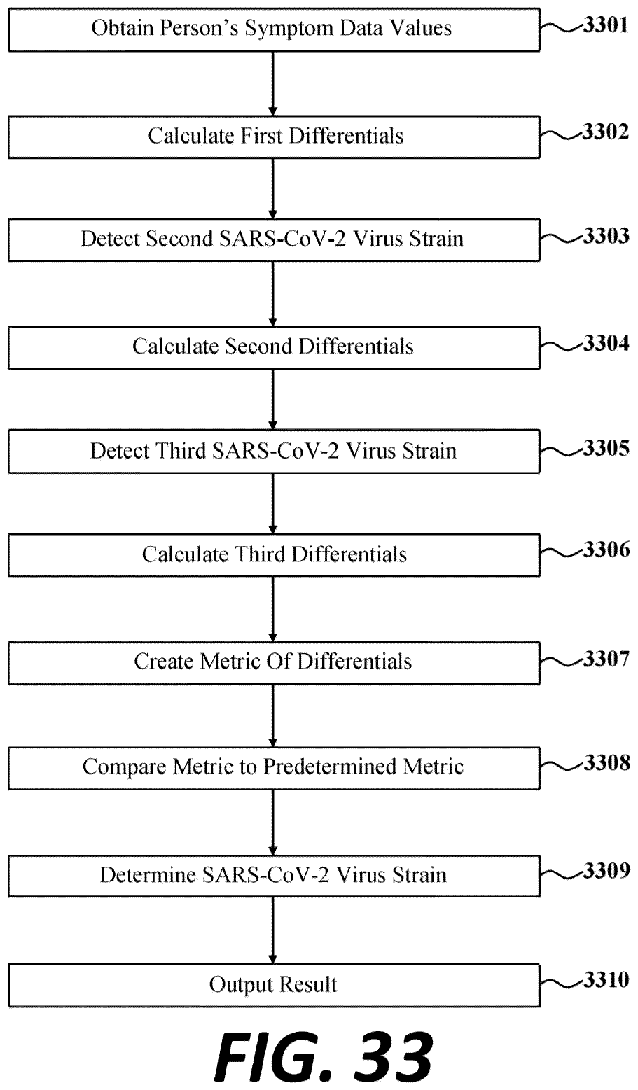

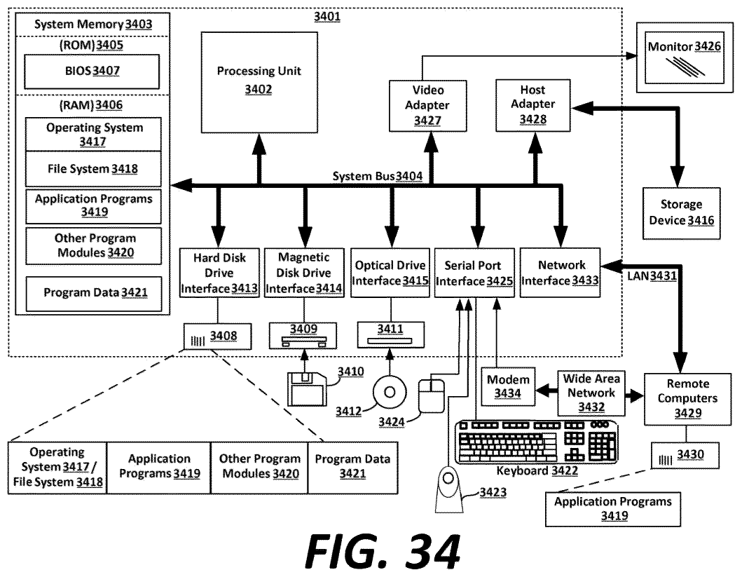

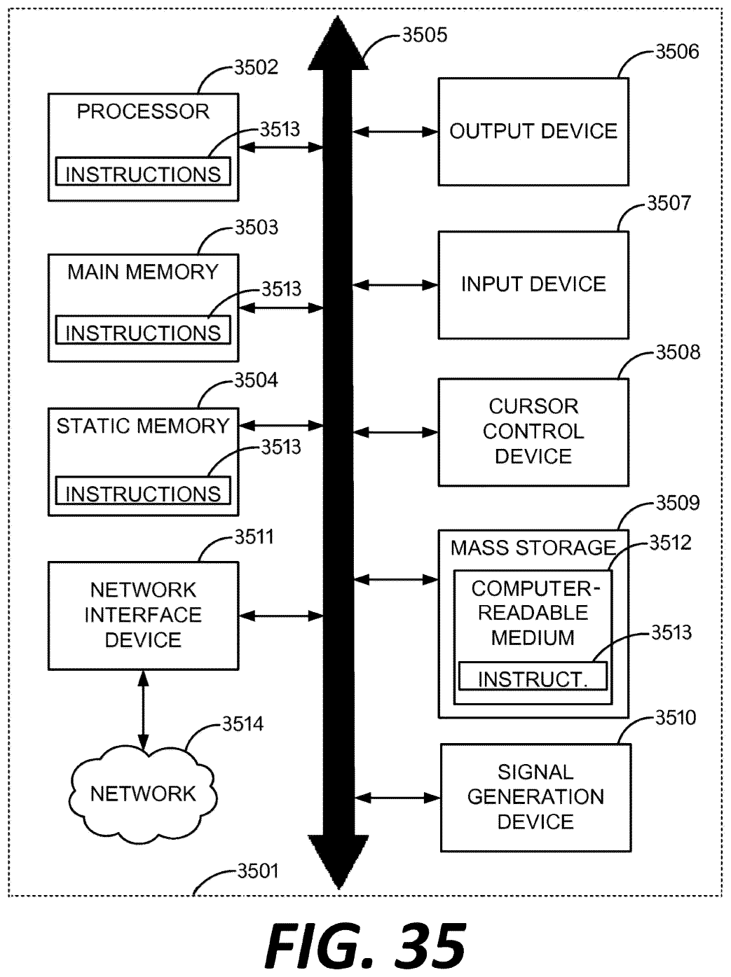

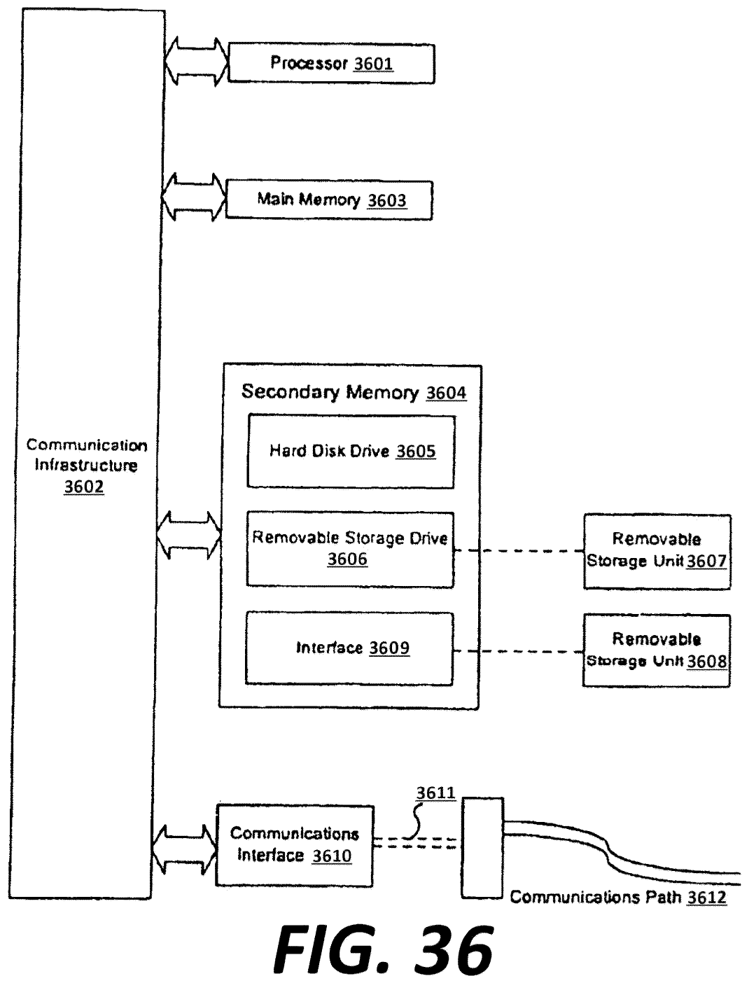

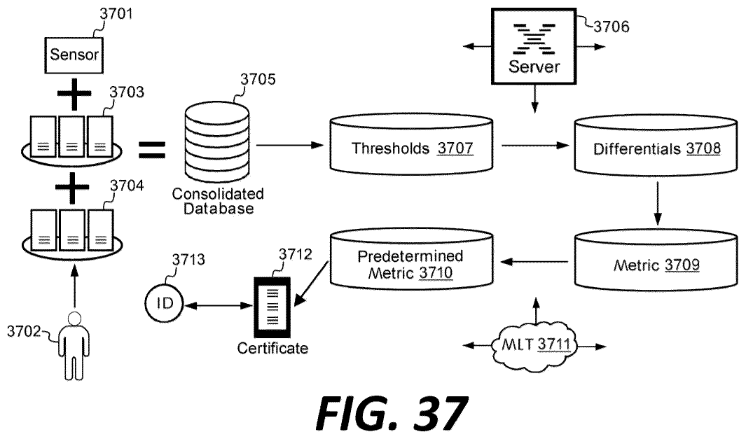

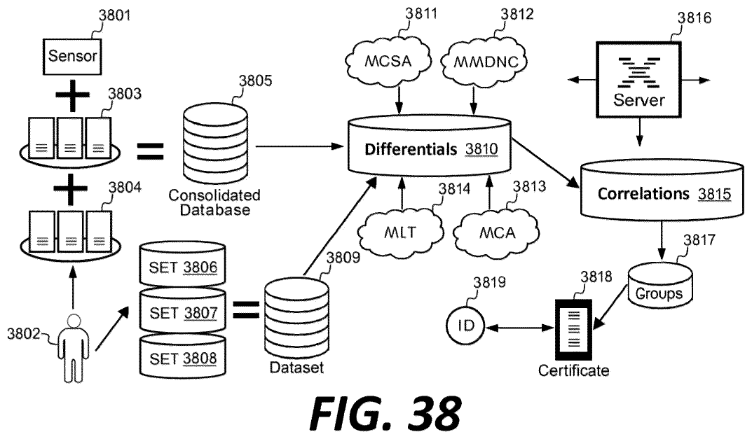

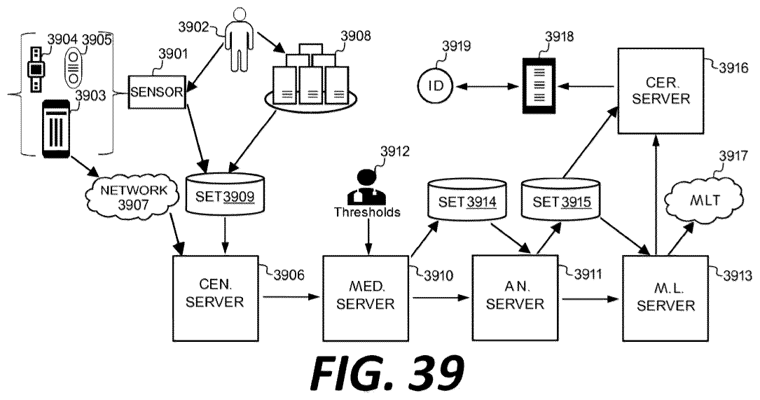

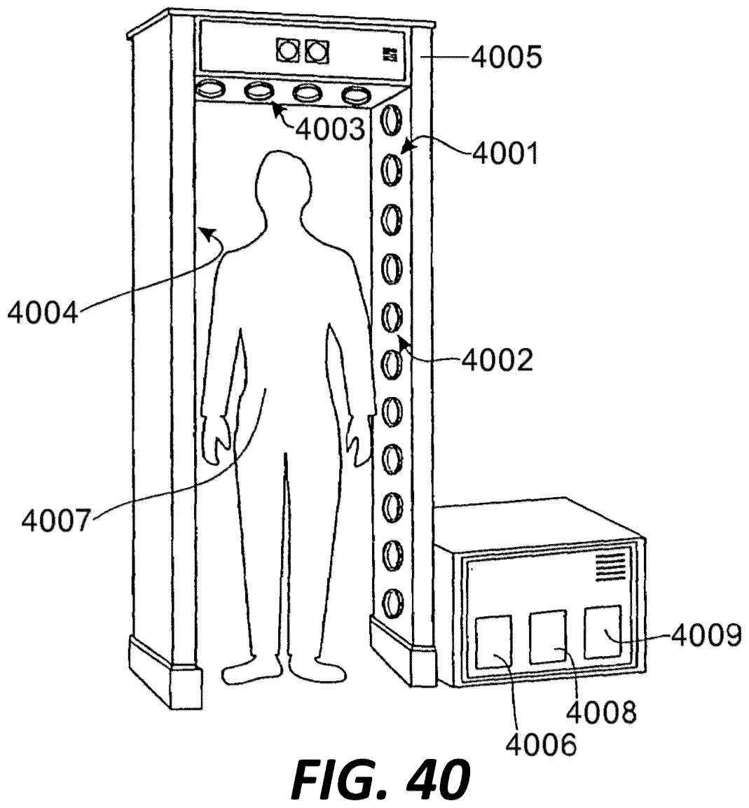

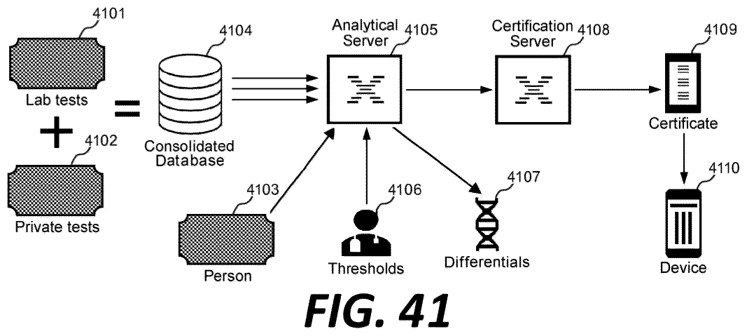

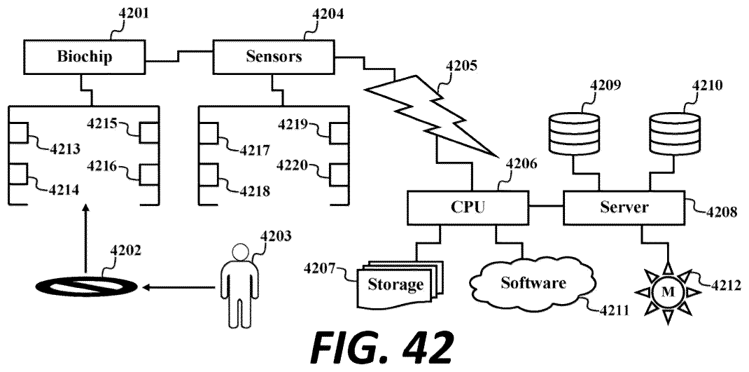

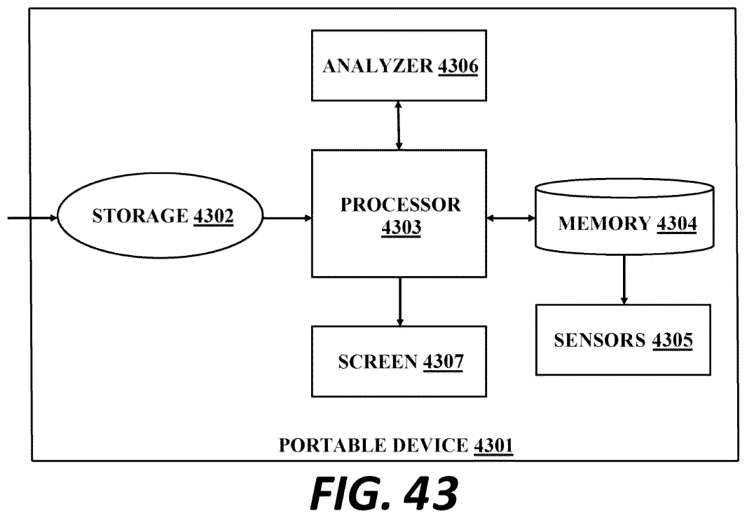

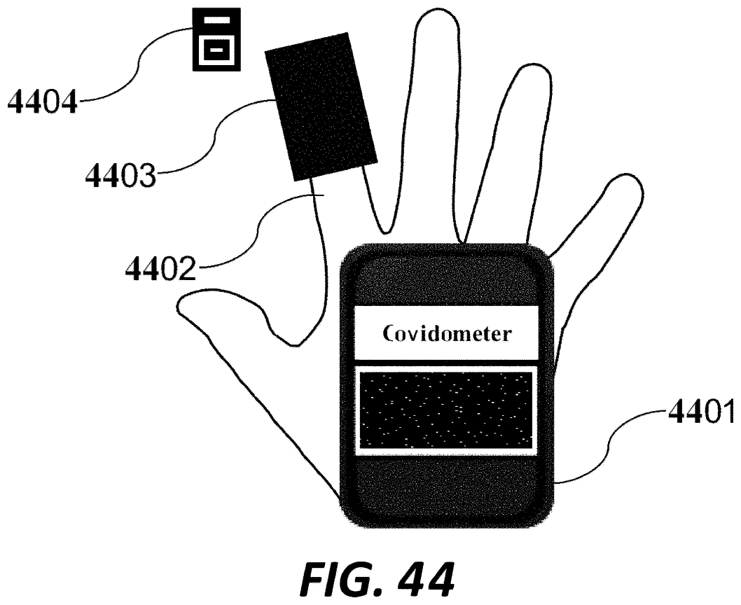

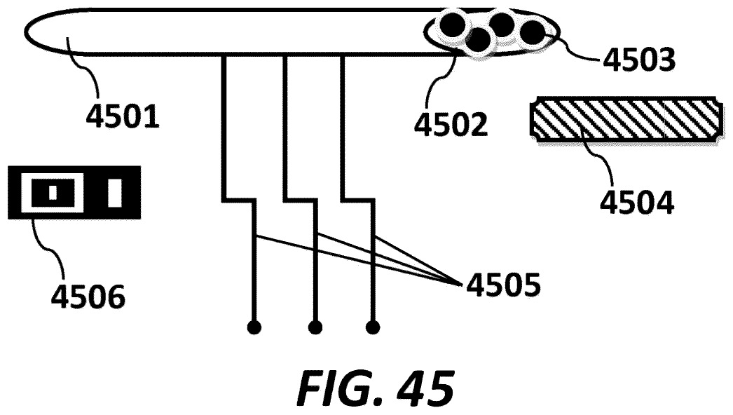

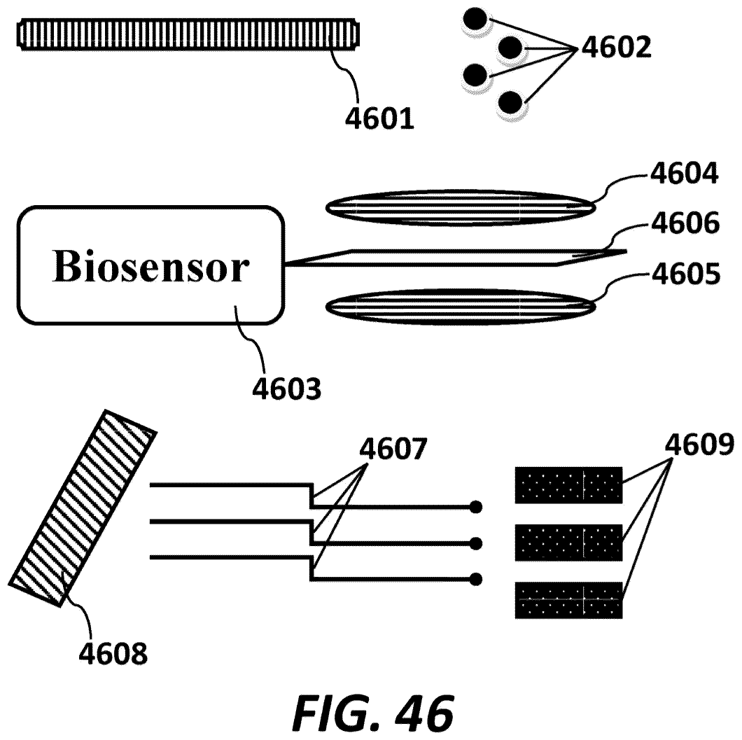

FIG. 29 is a diagram illustrating hardware components for implementing the regression data analysis.

FIG. 30 illustrates a graphical example of using the regression data analysis.

FIG. 31 is a flowchart illustrating the steps of the algorithm according to a first embodiment of the invention.

FIG. 32 is a flowchart illustrating the steps of the algorithm according to a second embodiment of the invention.

FIG. 33 is a flowchart illustrating the steps of the algorithm according to a third embodiment of the invention.

FIG. 34 is a diagram illustrating an example of the computer system for implementing the invention.

FIG. 35 is a diagram illustrating another example of the computer system for implementing the invention.

FIG. 36 is a diagram illustrating yet another example of the computer system for implementing the invention.

FIG. 37 is a diagram illustrating the system according to a first embodiment of the invention.

FIG. 38 is a diagram illustrating the system according to a second embodiment of the invention.

FIG. 39 is a diagram illustrating the system according to a third embodiment of the invention.

FIG. 40 is a diagram illustrating the system according to a fourth embodiment of the invention.

FIG. 41 is a diagram illustrating the system according to a fifth embodiment of the invention.

FIG. 42 is a diagram illustrating the system for detecting COVID variants implemented in the device of the “Covidometer.”

FIG. 43 is a diagram illustrating hardware components for implementing the device of the “Covidometer.”

FIG. 44 is a diagram illustrating an example of the operation of the device of the “Covidometer.”

FIG. 45 is a diagram illustrating another example of the operation of the device of the “Covidometer.”

FIG. 46 is a diagram illustrating yet another example of the operation of the device of the “Covidometer.”

### DETAILED DESCRIPTION OF THE PREFERRED EMBODIMENTS

Reference will now be made in detail to the preferred embodiments of the present invention, examples of which are illustrated in the accompanying drawings.

The invention relates to systems for detecting, analyzing, and diagnosing new COVID variants. Below are the main terms used in the present invention.

A virus is an infectious agent that replicates inside the living cells of an organism and infects all life forms, from plants and animals to humans. Examples of common human diseases caused by viruses include the common cold, influenza, chickenpox, and cold sores. Many serious diseases such as rabies, Ebola virus disease, AIDS (HIV), avian influenza, and SARS are caused by viruses. Viruses spread in many ways. Many viruses, including influenza viruses, SARS-CoV-2, chickenpox, smallpox, and measles, spread in the air by coughing and sneezing.

Coronaviruses are a group of related RNA viruses that cause severe acute respiratory syndrome diseases. COVID-19 is a viral disease caused by severe acute respiratory syndrome coronavirus 2 (SARS-CoV-2). The COVID-19 is the original SARS-CoV-2 virus strain, which is the base for new SARS-CoV-2 virus strains having a changed (mutated) virus genome code.

Major SARS-CoV-2 virus strains include the SARS-CoV-2 virus strains of concern currently recognized by the World Health Organization, SARS-CoV-2 virus strains of interest which are or were recognized by the World Health Organization, other notable SARS-CoV-2 virus strains. SARS-CoV-2 virus strains of concern—Alpha (lineage B.1.1.7), B.1.1.7 with E484K, Beta (lineage B.1.351), Gamma (lineage P.1), Delta (lineage B.1.617.2). SARS-CoV-2 virus strains of interest—Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529). Other notable SARS-CoV-2 virus strains—Lineage B.1.1.207, Lineage B.1.1.317, Lineage B.1.616, Lineage B.1.618, Brazilian variant, Centaurus variant, Deltacron variant.

Predetermined symptom threshold values for SARS-CoV-2 virus strains are predetermined actual limits for specific symptoms provided in medical literature, which, when exceeded, show that the person has been infected by a COVID disease. Medical guidelines are well established, documented in medical literature, and famous scientific facts, which indicate predetermined symptom threshold values for SARS-CoV-2 virus strains.

A differential is a positive or negative difference between the values of the patient's biochemical and biophysical data obtained and predetermined symptom threshold values for SARS-CoV-2 virus strains.

Correlation is any mathematical or logical relationship (dependence) between two random variables that is based on causation. A tendency is a special case of correlation and shows a possible direction among random variables.

Sensors include the devices which collect the patient's biochemical and biophysical data for detecting respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

Biosensors are analytical devices which convert a biological response into an electrical, magnetic or optical signal and combine a biological component with a physicochemical detector. The sensitive biological element, e.g., tissue, microorganisms, organelles, cell receptors, enzymes, antibodies, nucleic acids, etc., is a biologically derived material or biomimetic component that interacts with, binds with, or recognizes the analyte (the blood or sputum sample) under study.

Laboratory medical tests include the reverse transcription polymerase chain reaction (RT-PCR) test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay, etc. Laboratory medical examinations include chest CT scans, checking for elevated body temperature, checking for low blood oxygen levels, etc.

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural and logical changes may be made without departing from the spirit and scope of the present invention. The following detailed description is, therefore, not to be taken in a limiting sense, and the scope of the present invention is defined by the appended claims and their equivalents.

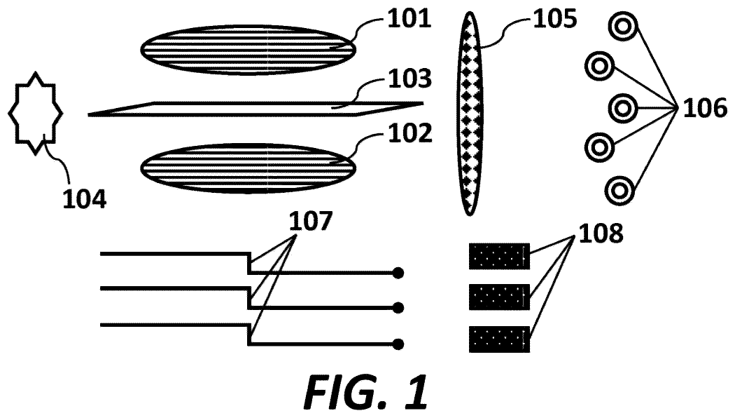

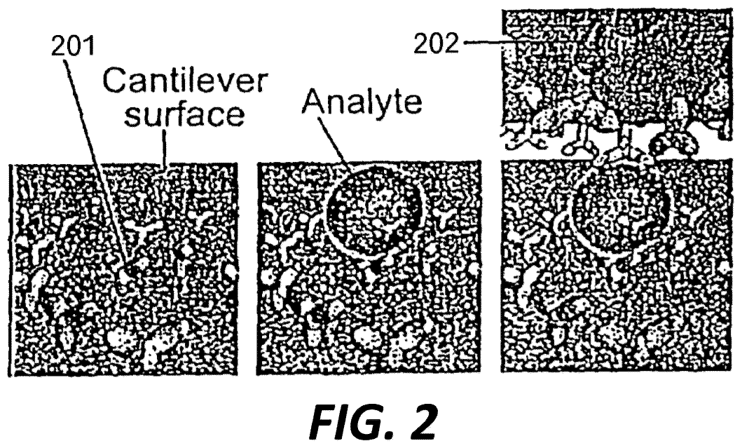

FIGS. 1-2 illustrate components and the operation of the biosensor utilizing two different biological components for implementing the present invention. In general, a biosensor is an analytical device which converts a biological response into a signal and combines a biological component with a physicochemical detector. The biologically responsive material, e.g., proteins (or protein structures), aptamers, lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc., is a biomimetic component that interacts with, binds with, or recognizes the analyte (blood or sputum sample) under study. The types of biological blood samples of a person that can be used for collection of the person's symptom data values may include a whole blood, blood plasma, serum samples, isolated antibodies, blood compositions, blood substitutes. The sputum samples include a nasal swab, nasopharyngeal swab, oropharyngeal swab, throat swab, deep airway material, saliva.

The types of biosensors of the present invention use the chemical or biological agent that generates a reaction of: 1) heat output (or heat absorption) by the reaction (calorimetric biosensor), 2) changes in distribution of charges causing an electrical potential to be produced (potentiometric biosensor), 3) movement of electrons produced within a redox (reduction oxidation) reaction (amperometric biosensor), 4) light output during a reaction or a light absorbance difference between the reactants and products (optical biosensors), 5) effects observed due to the mass of the reactants (piezo electric biosensor).

It will be understood that various other types of biological or chemical sensors may be employed within the scope of the present invention. For example, the biosensors can produce an electrical signal detectable by the sensors capable of distinguishing IgM and IgG antibodies from each other (e.g., a graphene-based sensors or electrochemical immunosensors capable of distinguishing IgM and IgG antibodies from each other.) In some aspects, the signal may be magnetic or optical (e.g., fluorescent emission.) If a biosensor outputs a magnetic signal as data, then sensors based on the atomic magnetometer (AM) or ion drift sensor are used to receive and register it. Optical sensors (e.g., a spectrometer or a fluorescence microscope) are used to register and analyze the fluorescent signal.

Sensors of present invention used to detect electrical, magnetic or optical signals or perform electrical, magnetic or optical measurements within the blood or sputum sample are electrochemical immunosensors, atomic magnetometer-based sensors, spectrometers, fluorescence microscopes, graphene-based sensors, ion drift sensors. Sensors of present invention used to detect characteristics of temperature or relative molecular motion or perform measurements of temperature or relative molecular motion within the blood or sputum sample are molecular electric transducers, oscillator-based sensors, flame ionization sensors, spectrometers, graphene-based sensors, ion drift sensors. Those skilled in the art will recognize that other sensors, not limited to those listed above, may also be used.

An electrochemical immunosensor uses an immunological reaction to determine the concentration of a certain substance in a sample. An electrochemical immunosensor combines the principles of immunology and electrochemistry, where an immunological reaction (for example, the binding of an antigen to an antibody) leads to a change in electrical properties.

An atomic magnetometer-based sensor measures extremely weak magnetic fields. An atomic magnetometer measures the magnitude of a magnetic field by measuring the polarization vector of an atomic spin in an external magnetic field.

Graphene-based sensors use graphene to measure various physical quantities such as humidity, blood glucose levels, pressure, and others. Graphene with defects can be used to create electrochemical sensors. Magnetic sensors have also been created on its basis.

The ion-drift sensor is used to detect and determine the amount of various gaseous substances, including explosives. The ion-drift sensor operates on the principle of ion mobility spectrometry, providing detection of vapors of substances.

A molecular electric transducer uses molecules to control electric current. The molecular electric transducer is based on the use of molecules that can change their electron conductivity under the influence of external factors, such as voltage, light or chemicals.

Oscillatory sensors (or sensors with oscillating elements) use oscillatory processes to measure various physical quantities or generate signals. For example, a change in the capacitance of a capacitor caused by a change in a physical quantity (e.g. pressure, position, displacement) leads to a change in the oscillator frequency.

A flame ionization sensor is a metal electrode whose operation is based on the ionization effect of gases when they pass into a plasma state during combustion. For example, an ionization electrode measures the ion current passing through a flame.

A spectrometer is used to measure the frequency and density of radiation, as well as to measure the spectra of electromagnetic radiation. The main function of a spectrometer is to record and accumulate a light spectrum, digitize the received signal depending on the wavelength and then analyze it.

A fluorescence microscope determines and details fluorescent colors, color brightness and purity, color saturation and saturation. A fluorescence microscope uses the method of luminescence of excited atoms when studying biological samples, which creates the phenomenon of fluorescence in the form of visible light with a longer wavelength.

The present invention proposes to use specially developed the biosensor utilizing two different biological components. Biosensors of the present invention have two different biological components (e.g., proteins (or protein structures), aptamers, lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc.) that are separated by an internal membrane and coupled to a transducer or amplifier. Common to all types of biosensors utilizing two different biological components are recognition elements used as the first or second biological component: proteins, immunoglobulins (antibodies), enzymes (or homogenates of microbial cells), nucleic acids (DNA, RNA, PNA), microbial cells (microorganisms) and aptamers (short DNA and RNA oligonucleotides capable of specifically binding to certain target molecules.)

As shown in FIG. 1, the biosensor utilizing two different biological components, where the first biological component 101 includes proteins or aptamers. The first biological component 101 and second biological component 102 are separated by an internal membrane 103 (e.g., a semi-permeable membrane, multilayer graphene membrane, ceramic membrane, iron membrane, silicone membrane, Teflon membrane, polymer membrane, rubber membrane, glass membrane, quartz membrane, etc.) The first biological component and the second biological component are coupled to the same transducer 104 within the biosensor. The biosensor may include a microfluidic chip 105 that allows only small molecules of the blood or sputum sample to pass through to the biosensor and does not allow large molecules to pass through.

The first biological component (proteins (or protein structures) or aptamers) 101 and second biological component (e.g., lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc.) 102 interact with, binds with, or recognizes the blood or sputum sample (the analyte) under study. As a result of a change in the chemical composition of a blood or sputum sample, reactions are formed when the first and second biological components chemically interact with sample molecules 106. These reactions are determined and sensed by a transducer 104 which converts it to signals 107 (e.g., an electrical, magnetic, and/or optical signal). The sensors 108 will then receive, decipher and analyze the data on signals from the biosensor.

As shown in FIG. 2, for each SARS-CoV-2 virus strain to be identified, specific molecules will be identified. A protein 201 capable of recognizing a target substance will then be generated for each specific molecule and these proteins will be contained in the biosensor utilizing two different biological components. A sample will be delivered to the biosensor and moved past the proteins. The proteins 201 will bind to the target molecules to be identified in the blood or sputum solution. Thereafter, beads 202 will be brought past the biosensor. The beads 202 have covalently bound proteins that attach to the target molecules. The number of beads may be counted by sensors. The number of beads will indicate the concentration of the target molecules.

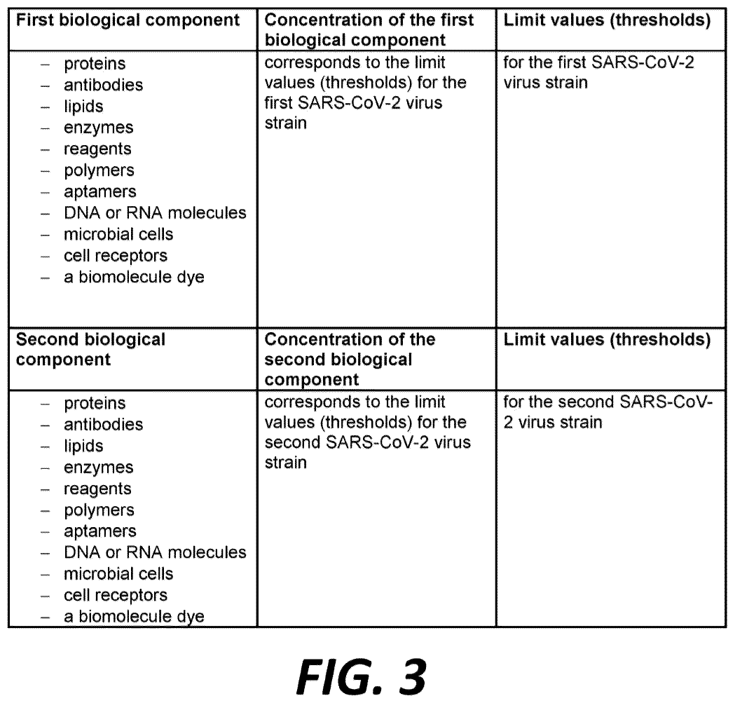

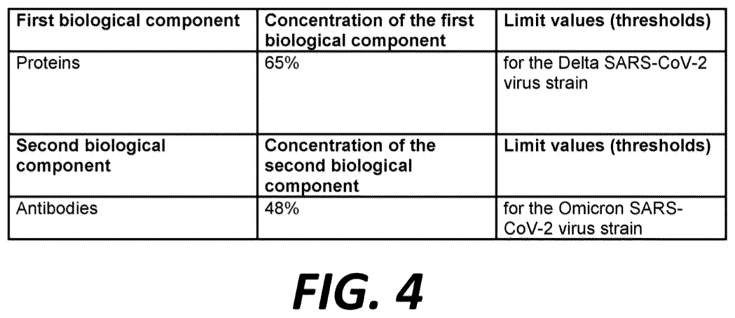

FIGS. 3-4 illustrate examples of the compositions of two different biological components within the biosensor. As noted above, the biosensor of the present invention uses two different biological components with the first and second biological components separated by an internal membrane (e.g., a semi-permeable membrane or multilayer graphene membrane membrane). These biological components are proteins (or protein structures), aptamers, lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye. When interacting with blood or sputum samples, the first biological component reacts chemically with the sample and changes the sample chemistry, and the second biological component also reacts chemically with the sample and also changes the sample chemistry.

As shown in FIG. 3, the concentration of each biological component (e.g., proteins (or protein structures), aptamers, lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye) is adjusted in such a way that when mixed with blood or sputum samples obtained from a person, this biological component enters into a chemical reaction with the sample and this chemical reaction is pronounced for fixation (e.g., by detectors), only if the sample contains the presence of the SARS-CoV-2 virus strain. Therefore, the concentration of the first biological component corresponds to the limit values (thresholds) for the first SARS-CoV-2 virus strain within the blood or sputum. Accordingly, the concentration of the second biological component corresponds to the limit values (thresholds) for the second SARS-CoV-2 virus strain within the blood or sputum.

FIG. 4 illustrates an example of a composition of the first and second biological components, which are separated from each other by an internal membrane inside the biosensor. The first biological component represents proteins. The concentration of proteins in relation to their absolute specific gravity (100%) in the entire composition is 65%. This concentration of proteins means that if the Delta SARS-CoV-2 virus strain (the first SARS-CoV-2 virus strain) is present in the blood or sputum sample, then the proteins, whose concentration is 65%, will enter into a chemical reaction with the sample. This reaction will be clearly expressed and will be recorded by detectors.

The second biological component represents antibodies. The concentration of antibodies in relation to their absolute specific gravity (100%) in the entire composition is 48%. This concentration of antibodies means that if the Omicron SARS-CoV-2 virus strain (the second SARS-CoV-2 virus strain) is present in the blood or sputum sample, then the antibodies, whose concentration is 48%, will enter into a chemical reaction with the sample. This reaction will be clearly expressed and will be recorded by detectors.

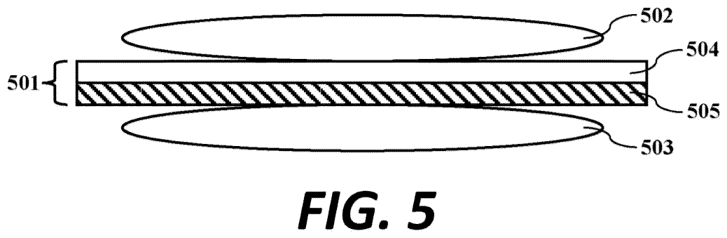

FIG. 5 illustrates an example of the cross-sectional view of an internal membrane that separates two biological components from each other within the biosensor of the present invention. The membrane 501 separates the first biological component 502 and the second biological component 503 within the biosensor. The membrane 501 is connected to a transducer inside the biosensor. The first biological component 502 and the second biological component 503 are connected to a transducer within the biosensor. The membrane 501 comprises one or more layers 504. The layers 504 may contain graphene, ceramic, iron, silicone, Teflon, polymer, rubber, glass, quartz. The membrane 501 also contains one or more semi-permeable layers 505. The semi-permeable layers 505 act as matrices to host the one or more layers 504.

A layer 504 may comprise graphene, ceramic, iron, silicone, Teflon, polymer, rubber, glass, quartz. In addition, the materials in the layer 504 may be an enzyme or oxidizing agent, for example. The layer 504 may be a separate layer within the membrane 501 or may be part of a semi-permeable layer 505. The one or more semi-permeable layers 505 may also be a layer 504 that is made of graphene, ceramic, iron, silicone, Teflon, polymer, rubber, glass, quartz, or contains additional material such as an enzyme or oxidizing agent.

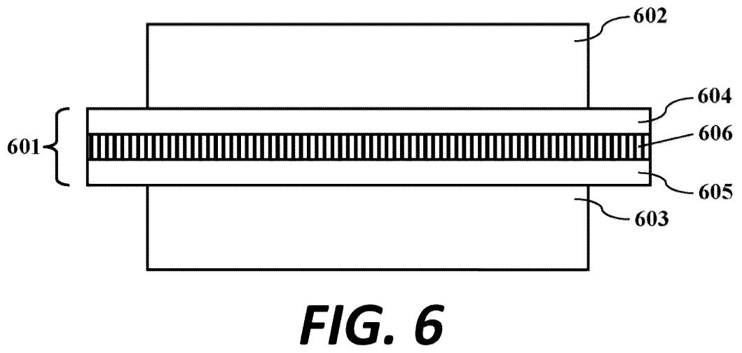

FIG. 6 illustrates another example of the cross-sectional view of an internal membrane that separates two biological components from each other within the biosensor of the present invention. A biosensor utilizing two different biological components separated by a multilayer membrane 601 within the biosensor comprises a contact surface 602 and a contact surface 603. The surface 602 of the membrane 601 contacts the first biological component of the biosensor. The surface 603 of the membrane 601 contacts the second biological component of the biosensor.

A multilayer membrane 601 contacts surfaces 602 and 603 and comprises layers 604 and 605. Layers 604 and 605 can be semi-permeable layers. Between layers 604 and 605 there is an optimum buffering zone 606. The buffering zone 606 may contain graphene, ceramic, iron, silicone, Teflon, polymer, rubber, glass, quartz, enzymes, or oxidizing agents. The combination of layers 604 and 605 can be specifically chosen to provide an optimum composition for buffering zone 606, for example, to increase the buffering species within the multilayer membrane 601.

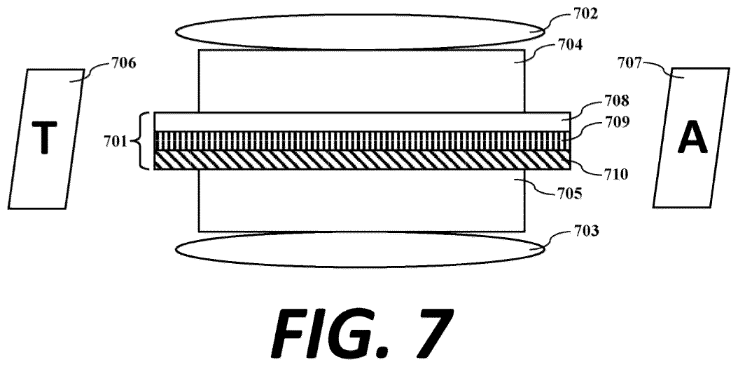

FIG. 7 illustrates a cross-sectional view of the multilayer semi-permeable membrane that separates two biological components from each other within the biosensor of the present invention. A protein biosensor contains an internal membrane 701 and two different biological components 702 and 703 separated from each other by a membrane 701 within the biosensor. The membrane 701 comprises two contact surfaces 704 and 705. The surface 704 of the membrane 701 contacts the first biological component 702 of the biosensor. The surface 705 of the membrane 701 contacts the second biological component 703 of the biosensor.

The membrane 701 separates the first biological component 702 and the second biological component 703 within the biosensor and comprises charged layers 707 and 708. A membrane 701 is in direct contact with a transducer 706, which transforms a biorecognition from analyte 707 (blood or sputum) into a signal, such as an electrical, magnetic, or optical signal. A signal is produced by the transducer 706 in response to chemical interaction of the first and second biological components 702 and 703 with the analyte 707 (blood or sputum).

The membrane 701 comprises polyelectrolyte or polymeric layers and conversion layers, which may be oppositely charged layers 708 and 709. A polyelectrolyte is a polymer whose repeating units bear an electrolyte group. Examples of polyelectrolytes are polysodium styrene sulfonate (PSS) and polyacrylic acid (PAA). Examples of materials which may be used to make the membrane include vinyl polymers having vinyl ester monomeric units. Natural polymers such as cellulosic and protein based materials, and mixtures or combinations thereof can also be used as the flux-limiting layer.

In one aspect, the material that comprises the membrane may be a vinyl polymer that allows relevant compounds to pass through it, for example, to allow an oxygen molecule to pass through in order to reach the active enzyme (the second biological component 703) or electrochemical electrodes (which can be located on the transductor 706 or the membrane 701 itself). In another aspect, the flux-limiting membrane substantially excludes condensation polymers such as silicone and urethane polymers and/or copolymers or blends thereof. Such excluded condensation polymers typically contain residual heavy metal catalytic material.

A conversion layer may comprise a conversion species. A conversion species may be an enzyme or oxidizing agent, for example. The conversion layer may be a separate layer from the polyelectrolyte layers or may be part of a polyelectrolyte layer. One or more of the polyelectrolyte layers may also be a conversion layer or comprise a conversion species. The alternating layers within the membrane 701 may be oppositely charged or create oppositely charged regions within membrane 701. For example, a positive conversion layer may be placed next to a positive polymeric layer (creating a positive region), which is then placed next to a negative polymeric layer.

The combination of polyelectrolyte layers and conversion layers can be specifically chosen to provide a buffering substance for the target biorecognition. One or more polyelectrolyte layers that alternate within the membrane 701 are purposely selected to create a buffering substance within the membrane 701. For example, polyelectrolytes may be chosen such that their pKa values are below the physiological condition and the operating pH of the protein biosensor. The polyelectrolytes pKa value may be two pH units or more below the physiological condition and operating pH of the protein biosensor.

In one embodiment, the buffering substance for blood analyte 707 is carbonate ion or can be a negatively charged polyelectrolyte membrane, such as a polyacrylic acid with a pKa around 4.5. The protein biosensor can detect the hydrogen ions produced by a reaction with glucose oxidase and operate within the pH range of about 5 to about 7.4. A strong negatively charged polyelectrolyte, such as polysodium sulfonate with a pKa of about 2, would decrease the buffering substance movement into the membrane 701 and therefore increase the signal output from transductor 706 of the protein biosensor.

In another embodiment, hydroxide ions are produced by the reaction with analyte 707 in a protein biosensor and the operating pH is around 7.4 to about 9. Polyacrylic acid could then be chosen to provide a buffering substance. By using a positively charged polyelectrolyte, such as polylysine, the membrane 701 would attract more buffering substance, such as carbonate, into the membrane and decrease the signal output from transductor 706 of the protein biosensor.

Thus, the membrane 701 can serve one or more functions including, for example, a) limiting of the flow of ions between the first biological component 702 and the second biological component 703; or b) reducing or eliminating the flux of interferents the first biological component 702 and the second biological component 703. A multilayer membrane 701 formed from an EVA polymer may serve as a flux limiter at the top of the membrane, but also serve as a sealant or encapsulant between the first biological component 702 and the second biological component 703.

The membrane 701 separating the first 702 and second 703 biological components within the protein biosensor includes at least three, and typically at least six, twelve, or eighteen layers. In some aspects, membrane 701 is formed using alternating polycationic and polyanionic layers. Typically, these layers are formed using polymers. Suitable polycationic polymers include, for example, polyallylamine hydrochloride (PAm), poly(4-vinylpyridine) quaternized by reacting about one third to one tenth of the pyridine nitrogens with 2-bromoethylamine (PVPEA), polyethylene imine, and polystyrene modified with quaternary ammonium functions. Suitable polyanionic polymers include, for example, poly(acrylic acid) (PAc), poly(methacrylic acid), partially sulfonated polystyrene, polystyrene modified with functions having carboxylate anions, and DNA (deoxyribonucleic acid) or RNA (ribonucleic acid) strands, fragments or oligomers.

The charged layers 708 and 709 can also include a conductive material. In some aspects, one 710 of the conversion layers within the membrane 701 is a graphene complex or compound as a conductive material, because graphene has excellent thermal conductivity properties in addition to unique electronic characteristics. The graphene complex or compound 710 may be incorporated or disposed only into or onto a portion of the membrane 701 adjacent to the interacting region with the analyte 707 (blood or sputum), or over the entire surface membrane 701. The graphene complex or compound 710 can be deposited in or on the membrane 701, for example, by coating, filling, solvent casting, or sorption of the graphene complex or compound 710 into the membrane 701.

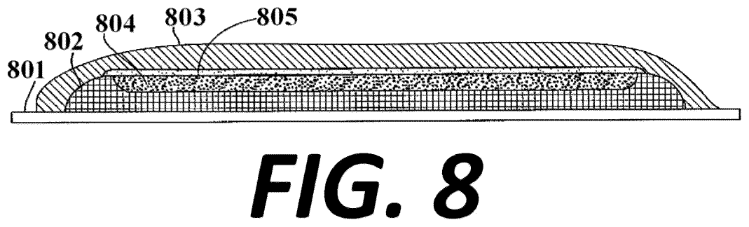

FIG. 8 illustrates a cross-sectional view of the semi-permeable graphene membrane that separates two biological components from each other within the biosensor of the present invention. The internal membrane of the present invention includes a porous polymer substrate 801 and a coating layer 802 formed on the porous polymer substrate 801 wherein the coating layer 802 is composed of graphene oxide. The porous polymer substrate 801 is made of a polymer selected from the group consisting of polysulfone, polyethersulfone, polyimide, polyetherimide, polyamide, polyacrylonitrile, cellulose acetate, cellulose triacetate, and polyvinylidene fluoride. The graphene oxide is functionalized graphene oxide prepared by the conversion of the hydroxyl, carboxyl, carbonyl or epoxy groups present in the graphene oxide to ester, ether, amide or amino groups.

The membrane may include a thin charged (selectively permeable) layer 802, e.g., porous graphene, on a porous (broadly permeable) substrate 801. A membrane may be prepared with thin charged layers 802 and 803, for example, ranging from about 500 angstroms to about 1 micrometers—as thin as possible, since resistance to the flow of ions from the first biological component to the second biological component or vice versa may scale linearly with membrane thickness.

The membrane comprises one or more active charged layers 802 and 803 of graphene or graphene oxide which can be bonded to a porous substrate 801. The charged layers 802 and 803 may be disposed on top of each other to minimize the uncovered area of the porous substrate 801 and may also beneficially mitigate defects present in the other active charged layers by covering them.

In an aspect, a membrane may include a porous substrate 801 and at least one charged layer 802 disposed on the porous substrate 801. The at least one charged layer 802 may include pores 804. In some aspects, a membrane may include a porous substrate 801 and at least one charged layer 802 disposed on the porous substrate 801. The at least one charged layer 802 may include pores 804 and may comprise at least one 805 of graphene and graphene oxide.

In another aspect, a membrane may include a porous substrate 801 and a first charged layer 802 disposed on the porous substrate 801. A second charged layer 803 may be disposed on the first charged layer 802. A plurality of pores 804 may be formed in the first 802 and second 803 charged layers, and the plurality of pores 804 may pass through both the first charged layer 802 and the second charged layer 803.

In yet another aspect, a membrane may include a porous substrate 801 and a first charged layer 802 disposed on the porous substrate 801. The first charged layer 802 may comprise at least one of graphene and graphene oxide 805. A second charged layer 803 may be disposed on the first charged layer 802 and may comprise at least one 805 of graphene and graphene oxide. A plurality of pores 804 may be formed in the first 802 and second 803 charged layers, and the plurality of pores 804 may pass through both the first charged layer 802 and the second charged layer 803.

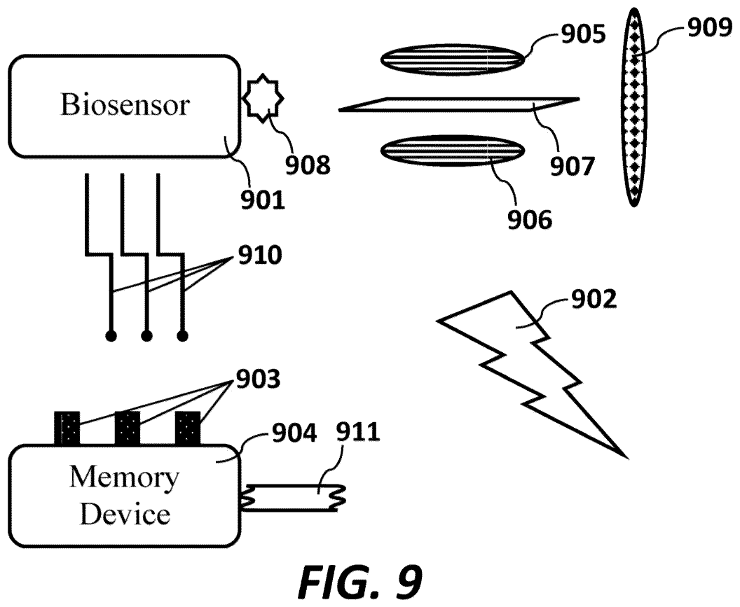

FIG. 9 is a diagram illustrating an example of the general system for detecting COVID variants of the invention. The system for determining COVID disease in a person comprises a protein biosensor 901 utilizing two different biological components that interacts with a blood or sputum sample obtained from a person; a wireless communication device 902 configured to communicate using a wireless peer-to-peer or machine-type-communication protocol; detectors 903 integrated into the memory device 904 that are configured to receive, decrypt and analyze data on signal from the biosensor 901 utilizing two different biological components; a memory device 904 coupled to the wireless communication device 902 and configured to determine signal thresholds for each detector 903 integrated into the memory device 904.

The detectors 903 integrated into the memory device 904 of the present invention may include electrochemical immunosensors, atomic magnetometers (AM), graphene-based sensors, ion drift sensors, molecular electric transducers (MET), oscillator-based sensors, flame ionization sensors, spectrometers, fluorescence microscopes, micro temperature sensors, motion sensors, conductivity sensors, electrical conductivity sensors, electrodermal activity (EDA) sensors, ECG sensors, EMG sensors, etc. The detector 903 integrated into the memory device 904 receive, decipher and analyze data from the protein biosensor 901. The protein biosensor 901, when interacting with the analyte (blood or sputum sample), enter into a chemical interaction with it, as a result of which they change the chemical structure of the analyte (blood or sputum sample). The change in the physical and chemical data as a result of chemical reactions with the analyte (blood or sputum sample), is recorded and received by detector 903 integrated into the memory device 904.

The biosensor 901 utilizing two different biological components is configured to include the first biological component 905 that represents proteins (or protein structures) or aptamers and the second biological component 906. The second biological component 906 within the biosensor 901 may be lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc. The first and second biological components are separated from each other by an internal membrane 907 (e.g., a semi-permeable membrane, multilayer graphene membrane, ceramic membrane, iron membrane, silicone membrane, Teflon membrane, polymer membrane, rubber membrane, glass membrane, quartz membrane.) The first and second biological components are coupled to the same transducer 908 within the biosensor 901.

The first biological component 905 within the biosensor 901 interacts with the blood or sputum sample and changes the chemical structure of the sample. The second biological component 906 within the biosensor 901 interacts with the blood or sputum sample and changes the chemical structure of the sample. The blood samples include a whole blood, blood plasma, serum samples, isolated antibodies, blood compositions, blood substitutes. The sputum samples include a nasal swab, nasopharyngeal swab, oropharyngeal swab, throat swab, deep airway material, saliva. In some aspects, the biosensor 901 includes a microfluidic chip 909 that allows only small sample molecules to pass through to the biosensor 901 and does not allow large molecules to pass through. In some aspects, the biosensor 901 uses graphene coatings for binding to the sample.

The concentration of the first biological component 905 corresponds to the threshold for the first SARS-CoV-2 virus strain, and the concentration of the second biological component 906 corresponds to the threshold for the second SARS-CoV-2 virus strain with a mutated virus genome code. The biosensor 901 outputs a first signal when the first biological component 905 changes the chemical structure of the blood or sputum sample, and outputs a second signal when the second biological component 906 changes the chemical structure of the sample.

Thus, the biosensor 901 output a first and second signals 910 (e.g., an electrical signals, magnetic signals, optical signals) when the first biological component 905 and second biological component 906 changes the chemical structure of the sample. The detectors 903 integrated into the memory device 904 then receive, decrypt and analyze the first and second signals 910 from the biosensor 901. In an aspect, the detectors 903 include electrochemical immunosensors or graphene-based sensors. In another aspect, the detectors 903 include sensors based on the atomic magnetometer (AM) or oscillator-based sensors. In yet another aspect, detectors 903 include spectrometers or fluorescence microscopes.

Each detector 903 integrated into the memory device 904 is embedded in the memory device 904 and coupled to the wireless communication device 902 via the detector output. The memory device 904 coupled to the wireless communication device 902 and configured to determine signal thresholds for each detector 903 integrated into the memory device 904, identify that the person has the first or second SARS-CoV-2 virus strain in response to the detector 903 are detecting a value that is greater than or less than the signal thresholds for each detector 903, and transmit an indication that the person has contracted the SARS-CoV-2 virus strain, via the wireless communication device 902, to another device.

In some aspects, the controller 911 is additionally installed to the memory device 904 and determines, by controller command decoder, the first signal threshold and the second signal threshold for each detector 903 integrated into the memory device 904. The memory device 904 transmits the indication to the wireless communication device 902 via the detector output responsive to a determination that a value detected by the detector 903 is greater than or less than the first signal threshold and responsive to a determination that a value detected by the detector 903 is greater than or less than the second signal threshold.

In some aspects, the controller 911 updates the first signal threshold and second signal threshold for each detector 903 integrated into the memory device 904 based on the detected change in the electrical, magnetic, and/or optical characteristics of the blood or sputum sample. In some aspects, the controller 911 updates the first signal threshold and second signal threshold for each detector 903 integrated into the memory device 904 based on the detected change in the temperature characteristic and characteristic of relative molecular motion of the blood or sputum sample (e.g., by using molecular electronic transducers (MET) as motion sensors integrated into a memory device.)

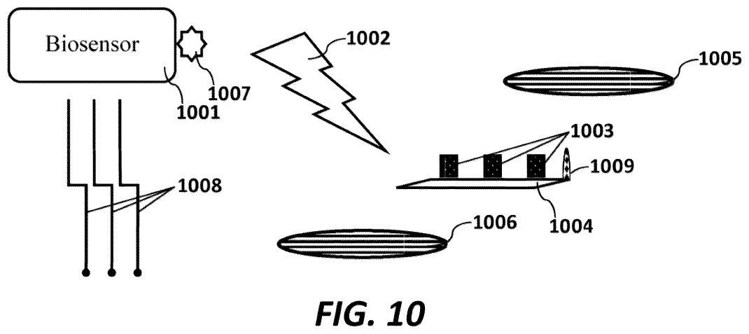

FIG. 10 is a diagram illustrating another example of the general system for detecting COVID variants of the invention. The system for determining COVID disease in a person comprises a protein biosensor 1001 utilizing two different biological components that interacts with a blood or sputum sample obtained from a person; a wireless communication device 1002 configured to communicate using a wireless peer-to-peer or machine-type-communication protocol; detectors 1003 integrated into the internal membrane 1004 that are configured to receive, decrypt and analyze data on signal from the biosensor 1001 utilizing two different biological components; an internal membrane 1004 separating the first and second biological components from each other within the biosensor 1001 and configured to determine signal thresholds for each detector 1003 integrated into the internal membrane 1004.

The detectors 1003 integrated into the internal membrane 1004 of the present invention may include electrochemical immunosensors, atomic magnetometers (AM), graphene-based sensors, ion drift sensors, molecular electric transducers (MET), oscillator-based sensors, flame ionization sensors, spectrometers, fluorescence microscopes, micro temperature sensors, motion sensors, conductivity sensors, electrical conductivity sensors, electrodermal activity (EDA) sensors, ECG sensors, EMG sensors, etc. The detector 1003 integrated into the internal membrane 1004 receive, decipher and analyze data from the protein biosensor 1001. The protein biosensor 1001, when interacting with the analyte (blood or sputum sample), enter into a chemical interaction with it, as a result of which they change the chemical structure of the analyte (blood or sputum sample). The change in the physical and chemical data as a result of chemical reactions with the analyte (blood or sputum sample), is recorded and received by detectors 1003 integrated into the internal membrane 1004.

The biosensor 1001 utilizing two different biological components is configured to include the first biological component 1005 that represents proteins (or protein structures) or aptamers and the second biological component 1006. The second biological component 1006 within the biosensor 1001 may be lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc. The first and second biological components are separated from each other by an internal membrane 1004 (e.g., a semi-permeable membrane, multilayer graphene membrane, ceramic membrane, iron membrane, silicone membrane, Teflon membrane, polymer membrane, rubber membrane, glass membrane, quartz membrane.) The first and second biological components are coupled to the same transducer 1007 within the biosensor 1001.

The first biological component 1005 within the biosensor 1001 interacts with the blood or sputum sample and changes the chemical structure of the sample. The second biological component 1006 within the biosensor 1001 interacts with the blood or sputum sample and changes the chemical structure of the sample. The blood samples include a whole blood, blood plasma, serum samples, isolated antibodies, blood compositions, blood substitutes. The sputum samples include a nasal swab, nasopharyngeal swab, oropharyngeal swab, throat swab, deep airway material, saliva. The biosensor 1001 outputs a first signal when the first biological component 1005 changes the chemical structure of the blood or sputum sample, and outputs a second signal when the second biological component 1006 changes the chemical structure of the sample.

Thus, the biosensor 1001 output a first and second signals 1008 (e.g., an electrical signals, magnetic signals, optical signals) when the first biological component 1005 and second biological component 1006 changes the chemical structure of the sample. The detectors 1003 integrated into internal membrane 1004 then receive, decrypt and analyze the first and second signals 1008 from the biosensor 1001. In an aspect, the detectors 1003 include electrochemical immunosensors or graphene-based sensors. In another aspect, the detectors 1003 include sensors based on the atomic magnetometer (AM) or oscillator-based sensors. In yet another aspect, detectors 1003 include spectrometers or fluorescence microscopes.

Each detector 1003 is embedded in the internal membrane 1004 within the biosensor 1001. The internal membrane 1004 configured to determine signal thresholds for each detector 1003 integrated into the internal membrane 1004, identify that the person has the first or second SARS-CoV-2 virus strain in response to the detector 1003 are detecting a value that is greater than or less than the signal thresholds for each detector 1003, and transmit an indication that the person has contracted the SARS-CoV-2 virus strain, via the wireless communication device 1002, to another device.

In some aspects, the controller 1009 is additionally installed to the internal membrane 1004 and determines, by controller command decoder, the first signal threshold and the second signal threshold for each detector 1003 integrated into the internal membrane 1004. The internal membrane 1004 transmits the indication to the wireless communication device 1002 via the detector output responsive to a determination that a value detected by the detector 1003 is greater than or less than the first signal threshold and responsive to a determination that a value detected by the detector 1003 is greater than or less than the second signal threshold.

In some aspects, the controller 1009 installed to the internal membrane 1004 updates the first signal threshold and second signal threshold for each detector 1003 integrated into the internal membrane 1004 based on the detected change in the electrical, magnetic, and/or optical characteristics of the blood or sputum sample. In some aspects, the controller 1009 installed to the internal membrane 1004 updates the first signal threshold and second signal threshold for each detector 1003 integrated into the internal membrane 1004 based on the detected change in the temperature characteristic and characteristic of relative molecular motion of the blood or sputum sample (e.g., by using molecular electronic transducers (MET) as motion sensors integrated into an internal membrane.)

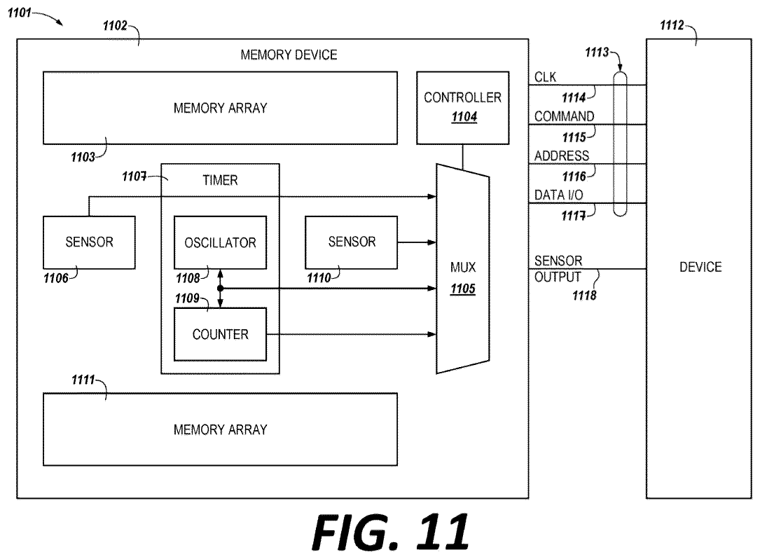

FIG. 11 is a diagram illustrating an example of components of the system with memory device sensors for implementing the invention. Experts in this area will recognize the sensors could also be incorporated and embedded into the internal membrane of the biosensor, similar to the technologies described in FIG. 11. A computing system 1101 that includes memory device 1102. The memory device 1102 can include memory array 1103 and memory array 1111 which may be collectively referred to herein as the memory array 1103/1111. The memory device 1102 can include a controller 1104 coupled to a multiplexer (MUX) 1105. The MUX 1105 can be coupled to one or more sensors embedded in circuitry of the memory device 1102. For example, the MUX 1105 can be coupled to an electrochemical immunosensor 1106, a timer 1107 (e.g., for self-refresh control), an oscillator (oscillator-based sensor) 1108, a counter 1109, and a sensor based on the atomic magnetometer (AM) 1110, which may be collectively referred to as the sensor or the sensors 1106/1110. Although specific types of sensors are mentioned herein, the present invention is not so limited and other sensors can be used (e.g., a graphene-based sensor, ion drift sensor, molecular electric transducer (MET), flame ionization sensor, spectrometer, fluorescence microscope, micro temperature sensor, motion sensor, conductivity sensor, electrical conductivity sensor, electrodermal activity (EDA) sensor, ECG sensor, EMG sensor, etc.)

The memory device 1102 can include volatile or non-volatile memory. For example, the memory media of the memory device 1102 can be volatile memory media such as DRAM. DRAM can include a plurality of sensors which can be at least one of an electrochemical immunosensor, a sensor based on the atomic magnetometer, an oscillator (oscillator-based sensor), a timer, or a combination thereof. The memory device 1102 can be coupled to another device 1112 via a bus 1113. The bus 1113 can include a clock line (CLK) 1114, a command line 1115 to transmit commands, an address line 1116 to determine where commands should be sent, and a data input/output (data I/O) 1117. The other device 1112 can be a central processing unit (CPU), a graphics processing unit (GPU), an application specific integrated circuit (ASIC), an edge computing device, etc. The other device 1112 can be a host and/or included as part of another device such as a workstation.

A host (e.g., a processor, a CPU, a computing system, etc.) can be a host system such as a processor within a wireless communication device, a processor within a personal laptop computer, a processor within a vehicle, a processor within a desktop computer, a processor within a digital camera, a processor within a mobile telephone, a processor within an IoT enabled device, or a processor within a memory card reader, a processor within graphics processing unit (e.g., a video card), among various other types of host systems. As used herein an “IoT enabled device” can refer to devices embedded with electronics, software, sensors, actuators, and/or network connectivity which enable such devices to connect to a network and/or exchange data.

The other device 1112 can include a system motherboard and/or backplane and can include a number of memory access devices, e.g., a number of processing resources (e.g., one or more processors, microprocessors, or some other type of controlling circuitry). One of ordinary skill in the art will appreciate that “a processor” can intend one or more processors, such as a parallel processing system, a number of coprocessors, etc. The other device 1112 can be coupled to memory device 1102 by the bus 1113. The controller 1104 can include a command decoder which can receive commands from the command line 1115 of the bus 1113. The command to read data from a sensor 1106/1110 can be received by the controller 1104. The command can be a mode register type command from the other device 1112 which can include information related to which sensor needs to output sensor data using the sensor output 1118. The MUX can be a device that selects between analog and digital input signals received from selection pins and forward the signal to the sensor output 1118.

As mentioned, the computing system 1101 includes a sensor 1106/1110 embedded in circuitry the memory device 1102. The sensor 1106/1110 can be configured to collect data related to the device 1112. For example, the device 1112 can be a part of and/or coupled to another device such as a workstation. The sensor 1106/1110 can be embedded in the memory device 1102 such as including memory such as DRAM and collect data corresponding to the electrical, magnetic and optical characteristics of the blood or sputum sample. Said differently, the embedded sensor 1106/1110 can be an electrochemical immunosensor 1106 which can generate a sensor data value (e.g., a particular electrical signal value) in the form of a temperature of the memory device 1102 coupled to another device such as a workstation. The memory device 1102 can be configured to transmit the sensor 1106/1110 data to the device 1112 using the sensor output 1118. For example, the sensor output 1118 coupled can be coupled to one or more of the sensors 1106/1110 and to the other device 1112 to transmit the sensor data collected by the sensor 1106/1110 to the other device 1102. The sensor output can be dedicated to the sensor embedded in the memory device 1102. In this way, embedded sensors 1106/1110 can be accessible by end applications to provide sensor generated sensor data.

In some aspects, the MUX 1105 can receive sensor data (electrical, magnetic and optical data of a sample) from multiple sensors 1106/1110 responsive to receiving a command from the controller 1105. For example, the controller 1105 can receive a request from the other device 1112 via the bus 1113 to read sensor data from one or more sensors 1106/1110. Responsive to receiving the request, the controller 1104 can transmit a command to the MUX 1105 to select and forward sensor data from the electrochemical immunosensor 1106 and the sensor based on the atomic magnetometer 1110, where the sensor based on the atomic magnetometer 1110 and the electrochemical immunosensor 1106 are both embedded in circuitry of the of the memory device 1102. The MUX 1105 can transmit the sensor data form the electrochemical immunosensor 1106 and the electrochemical immunosensor 1110 to the other device 1112 via the sensor output 1118. It will be understood by those skilled in the art that the sensors could also be embedded into the internal membrane of the biosensor and operate, similar to the technologies described in FIG. 11.

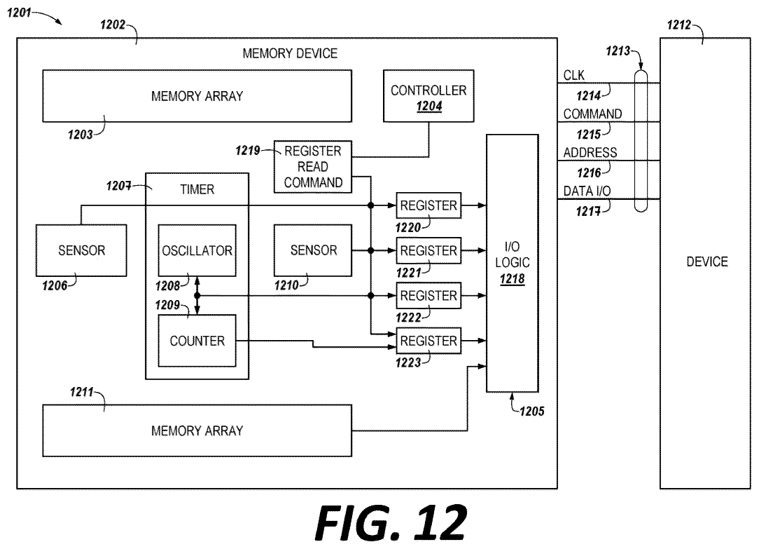

FIG. 12 is a diagram illustrating another example of components of the system with memory device sensors for implementing the invention. Experts in this area will recognize the sensors could also be incorporated and embedded into the internal membrane of the biosensor, similar to the technologies described in FIG. 12. A computing system 1201 includes memory device sensors 1206/1210 and includes memory device 1202 and be analogous to the memory device 1102 of FIG. 11. The memory device 1202 can include memory array 1203 and memory array 1211 which may be collectively referred to herein as the memory array 1203/1211 and be analogous to the memory array 1103/1111 of FIG. 11. The memory device 1202 can include controller 1204 which can be analogous to controller 1104 of FIG. 11.

The controller 1204 can be coupled to registers 1220, 1221, 1222, and 1223 and be collectively referred to herein as registers 1220-1223. The registers 1220-1223 can each be coupled to one or more sensors embedded in circuitry of the memory device 1202. For example, the register 1220 can be coupled to an electrochemical immunosensor 1206, the register 1221 can be coupled to a sensor based on the atomic magnetometer (AM) 1210, the registers 1222 and 1223 can be coupled to a timer 1207 via an oscillator (oscillator-based sensor) 1208 and/or a counter 1209, which may be collectively referred to as the sensor or the sensors 1206/1210. Although specific types of sensors are mentioned herein, the present invention is not so limited and other sensors can be used (e.g., a graphene-based sensor, ion drift sensor, molecular electric transducer (MET), flame ionization sensor, spectrometer, fluorescence microscope, micro temperature sensor, motion sensor, conductivity sensor, electrical conductivity sensor, electrodermal activity (EDA) sensor, ECG sensor, EMG sensor, etc.) that detected a change in the electrical, magnetic and optical characteristics of the sample or a change in the temperature characteristic and characteristic of relative molecular motion of the sample.

The memory device 1202 can be coupled to another device 1212 via a bus 1213. The bus 1213 can include a clock line (CLK) 1214, a command line 1215 to transmit commands, an address line 1216 to determine where commands should be sent, and a data input/output (data I/O) 1217. The other device 1212 can be a central processing unit (CPU), a graphics processing unit (GPU), an application specific integrated circuit (ASIC), an edge computing device, etc. The other device 1212 can be included as part of another device such as a workstation. The bus 1213 can be coupled to an input/output logic (I/O logic) 1218. The I/O logic 1218 can be a communication between the memory device 1202 and the other device 1212. The I/O logic 1218 can include hardware to perform input and output operations 1205 for the memory device 1202. The I/O logic 1218 can receive information (e.g., electrical, magnetic and optical data of a ample) from the imbedded sensors 1206/1210 and transmit them to the other device 1201 via the bus 1213.

FIG. 12 illustrates an example of another device 1212 and memory device 1202 coupled to the other device 1212. The memory device 1202 includes a plurality of sensors 1206/1210 embedded in the memory device 1202, and a plurality of registers 1220-1223 each respectively coupled to one of the plurality of sensors 1206/1210, the controller 1204 (e.g., a command decode) to transmit commands to read one or more of the plurality of registers, and a data output (data I/O) 1217 coupled to the plurality of registers 1220-1223 (e.g., via the I/O logic 1218) to transmit the sensor data from the plurality of registers 1220-1223 to the other device 1212. The signal (electrical, magnetic or optical signal) representing sensor data transmitted from the sensors 1206/1210 to respective registers 1220-1223 can be data of an operation of the sensor 1206/1210. This data can be used to detect change in the electrical, magnetic and optical characteristics of the ample. For example, the electrochemical immunosensor 1206 can generate an electrical signal value and transmit the electrical signal value to the register 1220, the embedded timer 1207 can include an oscillator (oscillator-based sensor) 1208 and/or a counter 1209 which can transmit a signal representing sensor data to register 1222 and/or 1223, the embedded sensor based on the atomic magnetometer 1210 can transmit magnetic signal value to the register 1221.

The embedded timer can include the oscillator (oscillator-based sensor) 1208 which can produce a periodic signal to transmit to the register 1222 and/or to the counter 1209. The counter 1209 can (independently or concurrently with the oscillator (oscillator-based sensor) 1208) transmit a quantity of incidences of sensor data collected by one or more of the sensors 1206/1210. Said differently, the oscillator (oscillator-based sensor) 1208 can work with the counter 1209 to periodically generate a signal which can report a quantity of sensor data signals generated from any of the sensors 1206/1210. In contrast, the oscillator (oscillator-based sensor) 1208 and the counter 1209 can operate independently to transmit respective sensor data to respective registers. In some aspects, the controller 1202 can configure the sensors 1206/1210 to generate sensor data based on the detected change in the electrical, magnetic and optical characteristics of the ample. For example, the controller 1204 can configure the sensors 1206/1210 to generate sensor data to the respective registers 1220-1223 when the other device 1212 is located in the blood or sputum sample. The controller 1204 can generate a register read command 1219 to read the sensor data stored in the respective registers and the I/O logic 1218 can transmit a signal representing sensor data from the registers 1220-1223 to the other device 1212.

The controller 1204 can receive an indication from the other device 1212 located in the blood or sputum sample, and the controller 1204 can configure the sensors 1206/1210 to generate sensor data about the change in the electrical, magnetic and optical characteristics of the sample. For example, the controller 1204 can receive an indication that the other device 1212 is located in the blood or sputum sample or the change in the temperature characteristic and characteristic of relative molecular motion of the sample. The controller 1204 can configure the electrochemical immunosensor 1206 to generate an electrical signal value (e.g., an encoded 8-bit binary string) and transmit the electrical signal value to the register 1220. Responsive to a register read command 1219 transmitted from the controller 1204, the I/O logic 1218 can transmit the sensor data from the register 1220 including the electrical signal value to the other device 1212. Said differently, the I/O logic 1218 can transmit the values related to the respective operations of the plurality of sensors 1206/1210 to the other device 1212. Using these methods, the electrical signal value generated by the embedded electrochemical immunosensor 1206 can be accessible to the other device 1212 and/or user.

In some aspects, the embedded timer 1207 (using an embedded oscillator (oscillator-based sensor) 1208 and/or an embedded counter 1209) can produce a timer output with a fixed period, for example, the controller 1204 may be configured to generate a register read command 1219 when a quantity of seconds have elapsed. The controller 1204 can program the memory device 1202 to generate sensor outputs to the respective registers 1220-1223 based on the quantity of seconds that have elapsed. As mentioned, the sensor based on the atomic magnetometer 1210 can be embedded in the circuitry of the memory device 1202 and can detect a change in magnetic signal within the sample.

The controller 1204 can receive an indication from the other device 1212 related to the detected change in the electrical, magnetic and optical characteristics of the sample, and the controller 1204 can configure the sensors 1206/1210 to generate sensor data about the detected change in the electrical, magnetic and optical characteristics of the sample. For example, the controller 1204 can receive an indication that the other device 1212 is located in the blood or sputum sample. The controller 1204 can configure the sensor based on the atomic magnetometer 1210 to generate a particular magnetic signal value is detected in the environment. Responsive to a register read command 1219 transmitted from the controller 1204, the I/O logic 1218 can transmit the sensor data from the register 1221 including the particular magnetic signal value to the other device 1212.

Similarly, the controller 1204 can receive an indication from the other device 1212 related to the detected change in the change in the temperature characteristic and characteristic of relative molecular motion of the sample (e.g., by using molecular electronic transducers (MET) as motion sensors embedded into a memory device.) The controller 1204 can then configure the sensors 1206/1210 to generate sensor data about the detected change in the temperature characteristic and characteristic of relative molecular motion of the sample.

In some aspects, multiple embedded sensors 1206/1210 can be used in combination to provide information to the user via the other device 1212. For example, the other device 1212 can be coupled to a wireless communication device which can initiate an operation responsive to transmission of the signal representing sensor data (e.g., from one or more of the sensors 1206/1210) from the plurality of registers 1220-1223 to the other device 1212. The wireless communication device can include the other device 1212 and can make decisions based on the received sensor data. For example, the wireless communication device may be a smartphone, and the other device 1212 coupled to the smartphone may receive an electrical signal value from the electrochemical immunosensor 1206, and the sensor based on the atomic magnetometer 1210 embedded in the memory device 1202 of the smartphone. Based on the receipt of the electrical signal value and the magnetic signal value, the other device 1212 may initiate the smartphone to change an operation. Using these methods, users can gain access to embedded sensor data and avoid the need for external sensor installations. It will be understood by those skilled in the art that the sensors could also be embedded into the internal membrane of the biosensor and operate, similar to the technologies described in FIG. 12.

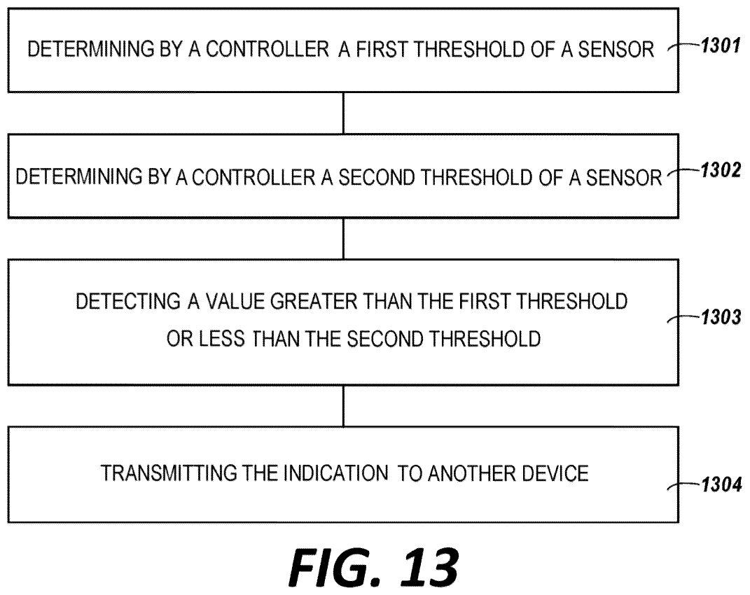

FIG. 13 is a flowchart illustrating the steps of using memory device sensors according to a first embodiment of the invention. In step 1301, a first threshold of a sensor (e.g., sensors 1106/1110 of FIG. 11) embedded in the memory device is determined by the controller (e.g., the controller 1104 of FIG. 11) coupled to a memory device (e.g., a memory device 1102 of FIG. 11) using the controller command decoder. The sensors can be embedded in the memory device and enabled to generate values (e.g., electrical signal values, magnetic signal values, optical signal values) which can be provided to another device and be accessible to users, or end applications. The sensor can be an electrochemical immunosensor embedded in the memory device and the first threshold can be a high electrical signal threshold. The controller can configure the electrochemical immunosensor to include a high electrical signal threshold and/or a low electrical signal threshold.

In step 1302, a second threshold of the sensor embedded in the memory device is determined by the controller using the controller command decoder. In this example, the second threshold can be a low electrical signal threshold. The controller can, via the memory device, transmit an indication to the other device responsive to the electrochemical immunosensor detecting an electrical signal that is greater than or less than the first threshold and the second threshold. In step 1303, the memory device transmits an indication responsive to the sensor detecting a value greater than the first threshold or less than the second threshold. In an aspect, the second threshold can be a low electrical signal threshold. To provide the other device and/or the workstation with the indication and/or the sensor data values from the embedded sensors, the memory device can transmit the indication (or sensor data values) via a sensor output dedicated to the embedded sensors of the memory device.

In step 1304, the indication is transmitted via a sensor output to another device. The other device may be a part of a workstation or a computing device that includes hardware and/or software to control the operations of the workstation. The other device can be directly or indirectly coupled to the sensors embedded in the memory device via the sensor output (e.g., the sensor output 1118 of FIG. 11). In some aspects, the memory device can alter the first and the second threshold of the embedded sensor based on the detected change in the electrical, magnetic, and optical characteristics of the sample. In some aspects, the memory device can alter the first and the second threshold of the embedded sensor based on the detected change in the temperature characteristic and characteristic of relative molecular motion of the sample.

For example, the system further is detected by the controller a change in the electrical, magnetic and optical characteristics of the sample, altering, by the controller, the first threshold of the sensor embedded in the memory device; and altering, by the controller, the second threshold of the sensor embedded in the memory device, where the first threshold and the second threshold are altered based at least in part on the detected change in the characteristics of the blood or sputum sample. The alteration of the sensor threshold can include disabling one or more sensors embedded in the memory device. While the examples of FIG. 13 describe the utilization of a sensor output, the present invention is not so limited. The examples, described in connection with FIG. 13 can utilize registers (e.g., registers 1220-1223 of FIG. 12) and an I/O logic (e.g., an I/O logic 1218 of FIG. 12). It will also be understood by those skilled in the art that the sensors could also be embedded into the internal membrane of the biosensor and operate, similar to the technologies described in FIG. 13.

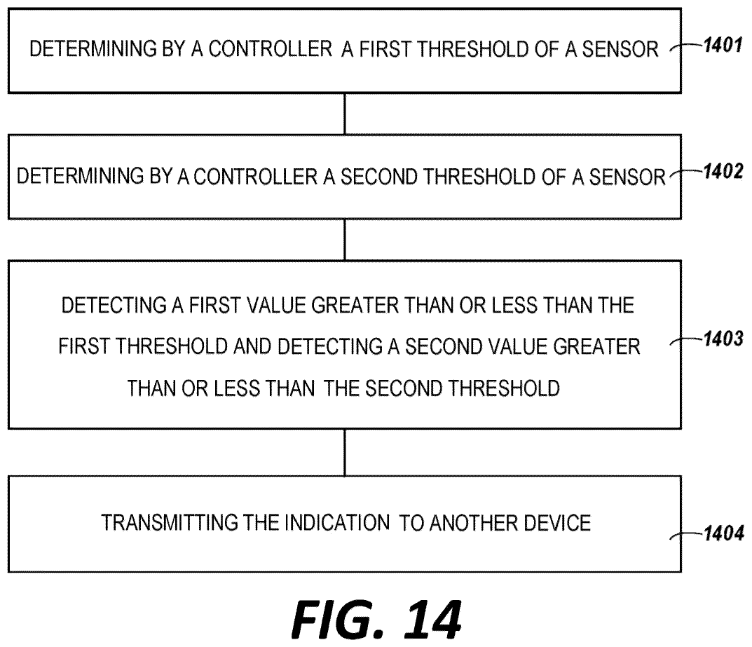

FIG. 14 is a flowchart illustrating the steps of using memory device sensors according to a second embodiment of the invention. In step 1401, a first threshold of a first sensor embedded in a memory device is determined by a controller using the controller command decoder. The sensors can be embedded in the memory device and enabled to generate values (e.g., electrical signal values, magnetic signal values, optical signal values s) which can be provided to another device and be accessible to users, or end applications. In step 1402, a second threshold of a second sensor (e.g., a sensor 1206/1210 of FIG. 12) embedded in the memory device is determined by the controller using the controller command decoder. The memory device and/or the controller included in the memory device can be configured to transmit an indication about the values (or the values themselves) generated by the sensors embedded in the memory device to the other device.

In step 1403, the memory device transmits an indication responsive to the first sensor detecting a first value greater than or less than the first threshold and responsive to the second sensor detecting a second value greater than or less than the second threshold. The indication can be transmitted via a sensor output. Using this method, the sensors embedded in the memory device can transmit the sensor data values generated to the other device coupled to the workstation, and/or an indication about the values detected by the embedded sensors can be transmitted to the other device coupled to the a workstation and/or another device.

For example, in step 1404, the indication is transmitted to another device, via a sensor output coupling the first sensor and the second sensor to the other device, wherein the indication is based on the first value and the second value. The indication can be an alert indicating that a value collected by the sensors embedded in the memory device is greater than or less than the respective configured thresholds. While the examples of FIG. 14 describe the utilization of a sensor output, the present invention is not so limited. The examples, described in connection with FIG. 14 can utilize registers (e.g., registers 1220-1223 of FIG. 12) and an I/O logic (e.g., an I/O logic 1218 of FIG. 12). While example of a workstation is used herein, other examples are contemplated. It will also be understood by those skilled in the art that the sensors could also be embedded into the internal membrane of the biosensor and operate, similar to the technologies described in FIG. 14.

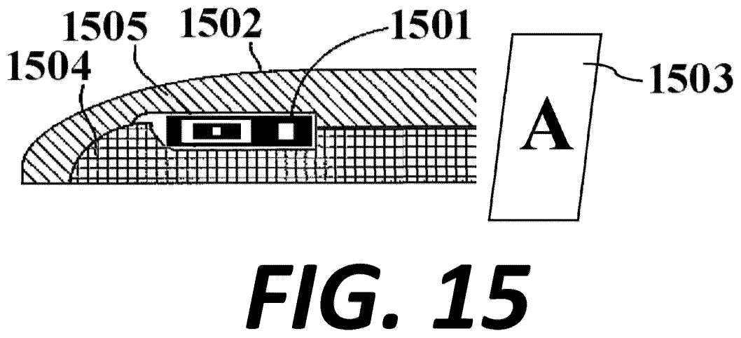

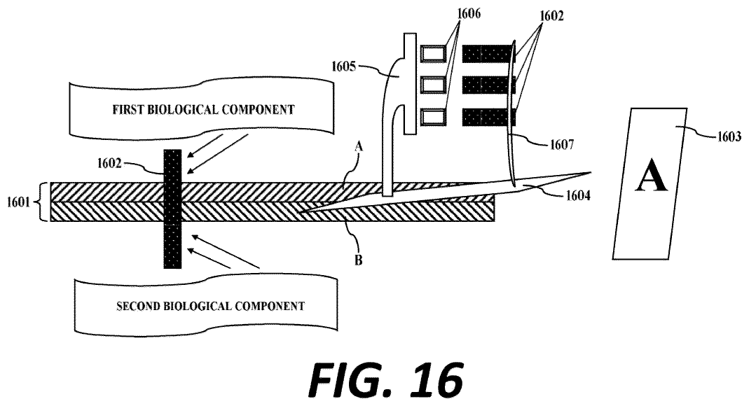

FIGS. 15 and 16 are diagrams illustrating examples of the detector embedded in the internal membrane for implementing the invention. As shown in FIG. 15, the detector 1501 is embedded inside the membrane 1502 which is located between the first biological component and the second biological component within the biosensor. When the biosensor interacts with the analyte (blood or sputum) 1503, the first and second biological components enter into a chemical reaction with the analyte, as a result of which the physical parameters of the first and second biological components change. The membrane with the detector embedded inside it contacts the first and second biological components, so the detector will directly record changes in the physical parameters and collect data from the first and second biological components.

For example, an ion drift sensor 1501 can be embedded inside the membrane 1502 and is configured to interact with one or more layers (semi-permeable layers, conversion layers, polyelectrolyte layers, polymer layers, graphene layers, etc.) within the membrane. When the conductivity of the membrane layer changes due to an increase in the number of ions passing through the semi-impermeable membrane layer from the first biological component to the second biological component due to the chemical interaction of the first and second biological components with blood or sputum 1503, the ion drift sensor will record the transition of ions through the membrane and measure their quantity. An increased amount of ions measured by the ion drift sensor embedded in the membrane may indicate that the threshold has been exceeded, which indicates the presence of the virus (the SARS-CoV-2 virus strain) in the blood or sputum sample.

The membrane 1502 is a multilayer membrane. A multilayer membrane comprises semi-permeable layers. One of the semi-permeable layers 1504 represents a polyelectrolyte (polyelectrolyte layer). A polyelectrolyte is a polymer (e.g., vinyl polymer). Another membrane layer 1505 is graphene or graphene complex (e.g., graphene oxide). The detector (ion drift sensor) 1501 can be deposited in or on the membrane 1502 by 1) placing a first membrane layer (vinyl polymer) 1504 by coating, filling, pouring with solvent or sorption, 2) installation of a detector 1501 on the first membrane layer 1504, 3) placing a second membrane layer (graphene oxide) 1505 on top of the detector 1501 by coating, filling, pouring with solvent or sorption. The detector 1501 may be incorporated or disposed only into or onto a portion of the membrane adjacent to the interacting region with the analyte (blood or sputum) 1503, or over the entire surface membrane. The detector 1501 may be covered with a special outer housing or film 1506, which may be formed from any suitable material (e.g., silicone) to ensure safe contact of the detector with the membrane layers 1504 and 1505.

FIG. 16 shows an internal membrane 1601 and two different biological components separated from each other by an internal membrane 1601 within the biosensor. The membrane 1601 comprises two contact surfaces. One surface of the membrane contacts the first biological component of the biosensor. Another surface of the membrane contacts the second biological component of the biosensor. The detector 1602 is built into an internal membrane 1601 that is located between the first and second biological components within the biosensor and prevents them from contacting each other. The membrane, with a detector built into it, serves as a physical barrier between the first and second biological components.

The detector 1602 collects data from the first biological component and the second biological component, since the membrane 1601 with the detector 1602 embedded therein is in contact with the first biological component and the second biological component within the biosensor. In some aspects, 1602 may be a set of detectors, which represent a sensor chip (several detectors that are connected to each other in a group) that is placed inside the membrane 1601. The sensor chip includes at least one hardware processor, memory, and wireless transmitter. Each detector within the sensor chip integrated into the membrane is coupled to the wireless transmitter that outputs, via direct, short range, wireless communication signals (e.g., Bluetooth), information from the detectors to another device (server).

The detector or sensor chip 1602 embedded in the membrane 1601 may include a electrochemical immunosensor, atomic magnetometer (AM), graphene-based sensor, ion drift sensor, molecular electric transducer (MET), oscillator-based sensor, flame ionization sensor, spectrometer, fluorescence microscope, micro temperature sensor, motion sensor, conductivity sensor, electrical conductivity sensor, electrodermal activity (EDA) sensor, ECG sensor, EMG sensor, etc. Detectors integrated into the membrane that separates the first and second biological components from each other receive, decipher and analyze data from the first biological component and the second biological component.

The first and second biological components, when interacting with the analyte (blood or sputum) 1603, enter into a chemical interaction with it, as a result of which they change the chemical structure of the analyte 1603. The change in the physical and chemical data of the first and second biological components, when they enter into chemical reactions with the analyte 1603, is recorded and received by detectors 1602 embedded into the membrane 1601, which contacts the first and second biological components.

The internal membrane 1601 represents a multilayer membrane and comprises semi-permeable layers, conversion layers, polyelectrolyte layers, polymer layers, graphene layers, etc. Embedding the detector 1602 into the internal membrane 1601 within the protein biosensor comprises the steps of: 1) disposing a detector or a group of detectors (a sensor chip) 1602 on a board 1604; 2) disposing and binding a first membrane layer A to one outer surface of the board 1604; 3) disposing and binding a second membrane layer B to another outer surface of the board 1604.

In one aspect, the detectors 1602 are directly embedded in the board 1604 disposed between the membrane layers A and B for receiving data from the first and second biological components that contact the membrane. In another aspect, a positioner (the sensor positioner) 1605 is built into the board 1604 and is configured to secure the detector(s) 1602 at least partially inside the membrane layers for receiving data from the first and second biological components that contact the membrane. The positioner 1605 comprises one or more connectors (caps) 1606 for fixing and protecting detectors 1602 to the positioner.

The board 1604 also comprises a controller 1607, which is configured to interact with layers within the membrane (semi-permeable layers, conversion layers, polyelectrolyte layers, polymer layers, graphene layers, etc.) and to receive data from the detectors 1602 embedded into the membrane. The controller 1607 is coupled to each of the detectors 1602 embedded into the membrane and is configured to set and update parameters for detectors 1602 based on detected changes within the analyte (blood or sputum) 1603 from interaction with the first and second biological components.

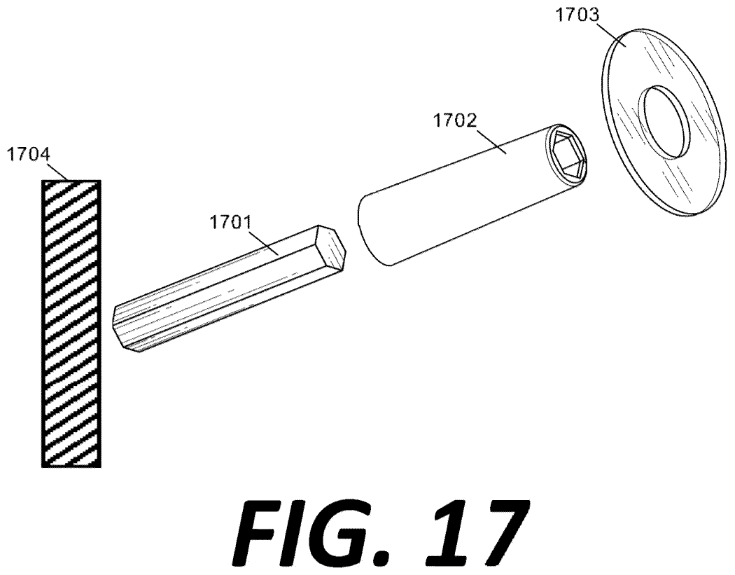

FIG. 17 illustrates a configuration of the positioner (sensor positioner) 1616 of FIG. 16 for disposing a detector to board in the internal membrane within the biosensor. The positioner includes a straight assembly 1701 and an outer housing 1702 with connector (cap) 1703 for fixing and protecting the detector. The positioner includes a straight assembly 1701 suitably sized and shaped for insertion into a membrane layer 1704 (a semi-permeable layer, conversion layer, polyelectrolyte layer, polymer layer, graphene layer, etc.) An outer housing 1702 is attached to the straight assembly 1701, surrounding and protecting the positioner with the detector located on it. The outer housing 1702 may be formed from any suitable material (e.g., silicone) to ensure safe contact with the membrane layer 1704.

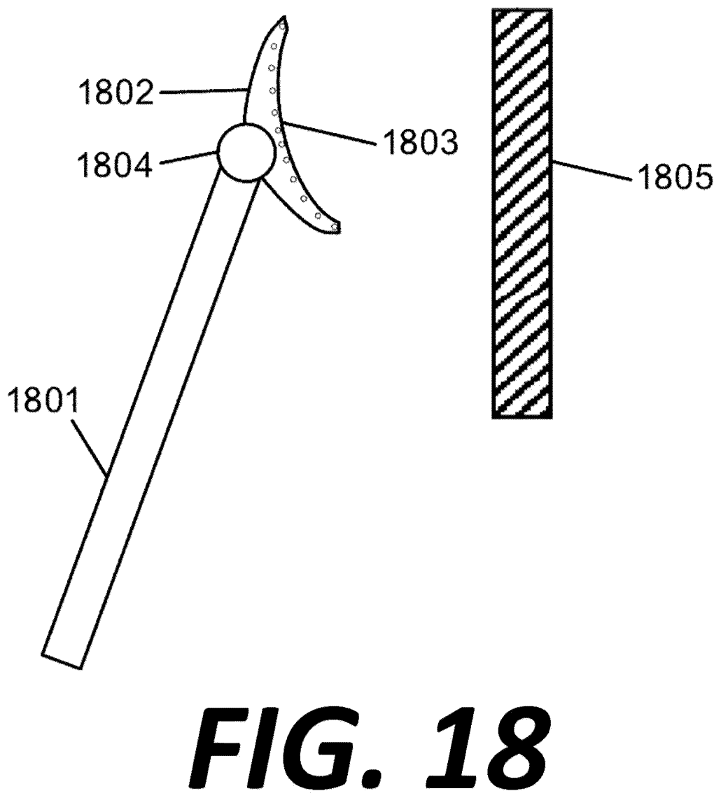

In the embodiment illustrated in FIG. 18, the positioner (sensor positioner) includes a body 1801 and a concave surface 1802 including one or more connectors (caps) 1803 for fixing and protecting detectors. The positioner includes a body 1801 that may be rigid, semi-rigid, or articulated. A joint 1804 may be arranged between body 1801 and a concave surface 1802. The concave surface 1802 has arranged therein or thereon the one or more connectors (caps) 1803, which may be encapsulated in membrane layers 1805 (semi-permeable layers, conversion layers, polyelectrolyte layers, polymer layers, graphene layers, etc.) and are designed to secure and protect detectors.

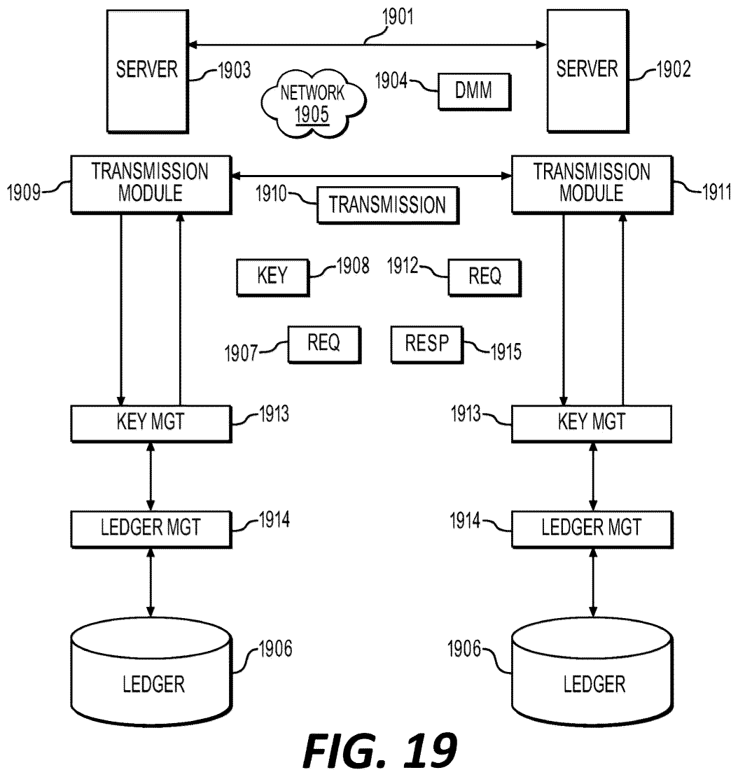

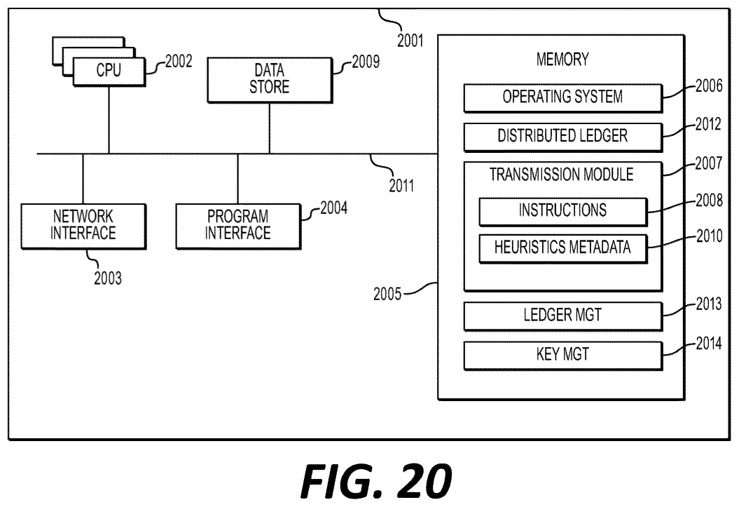

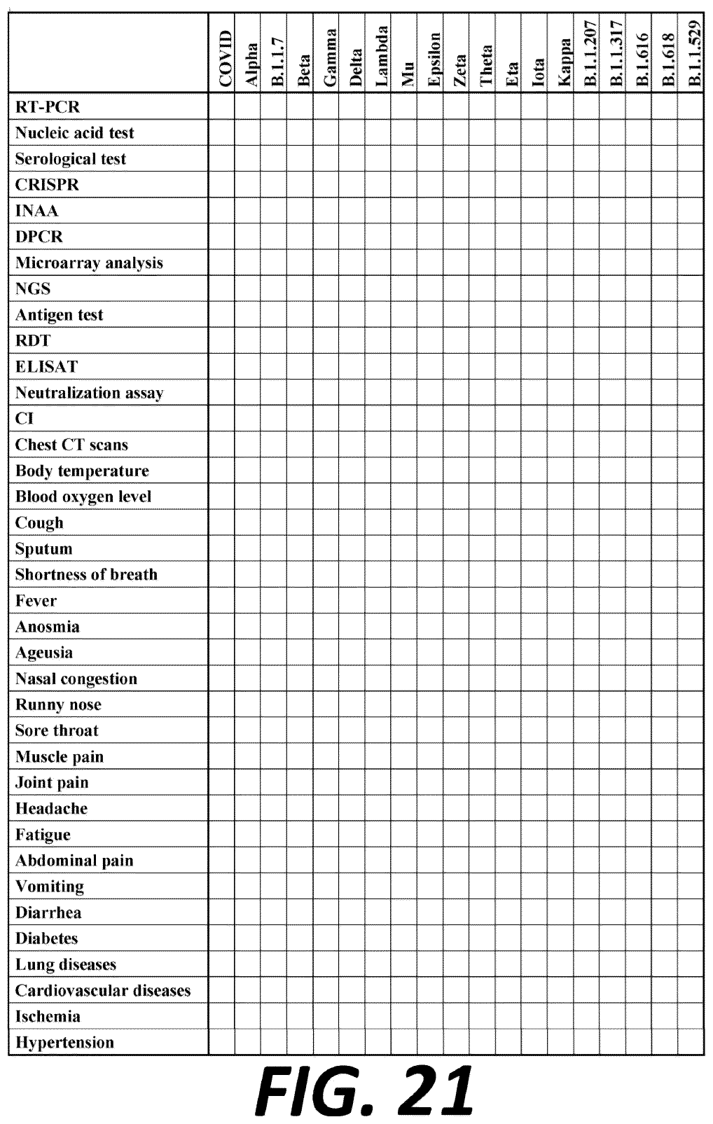

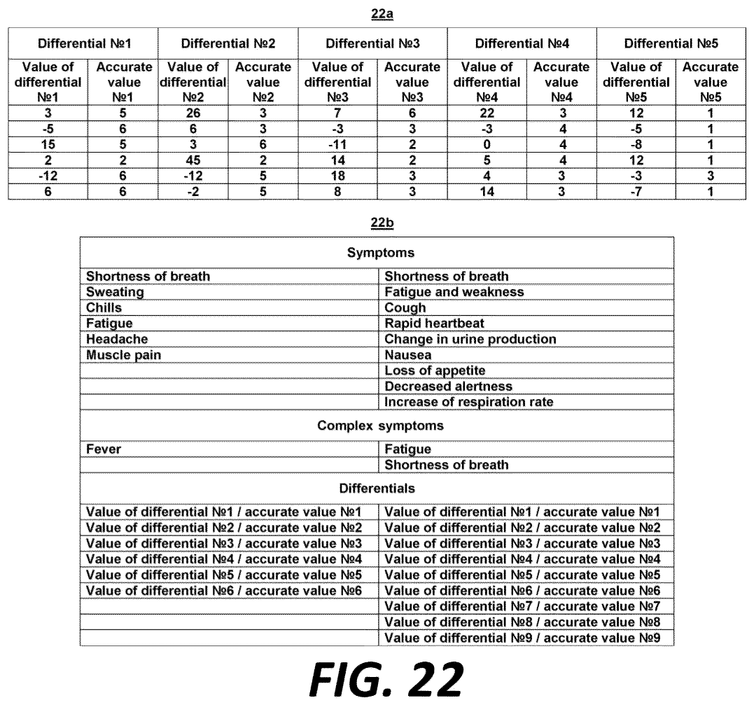

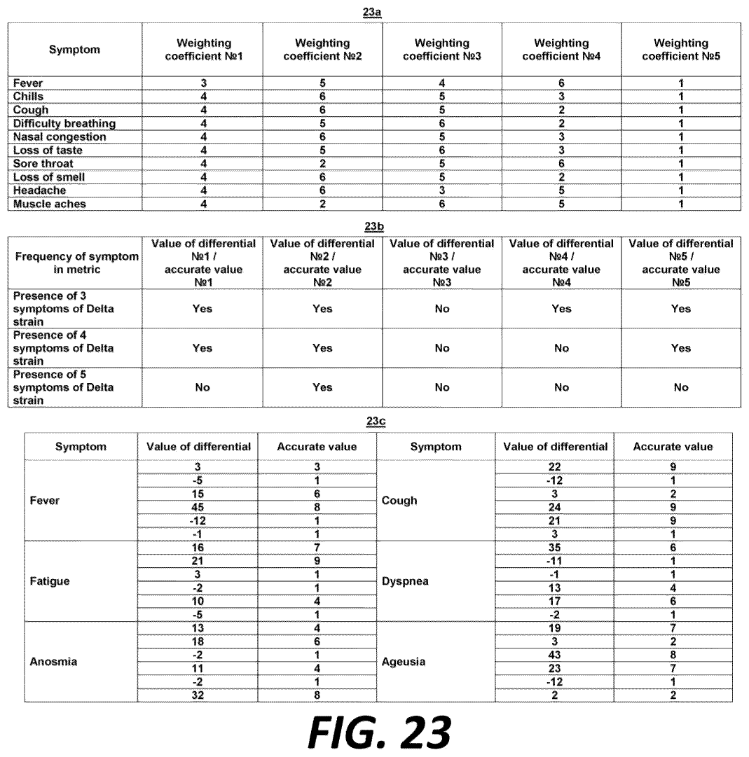

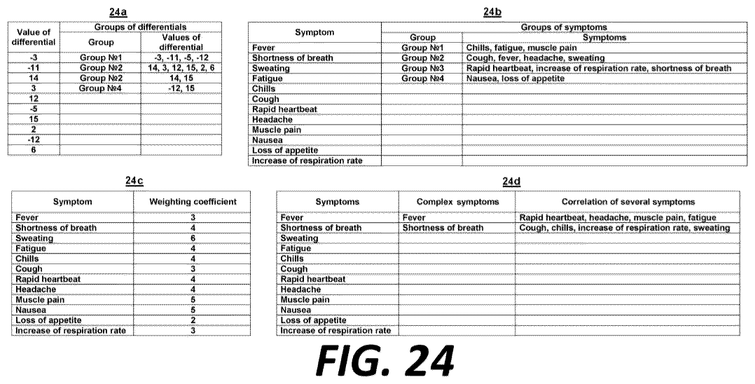

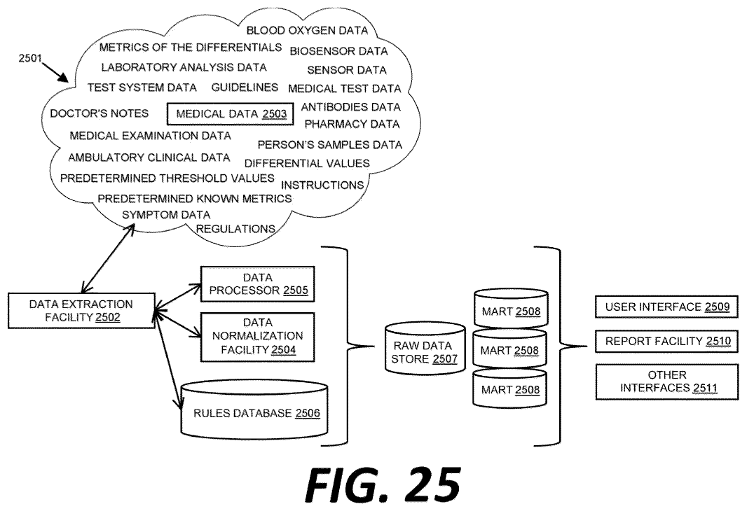

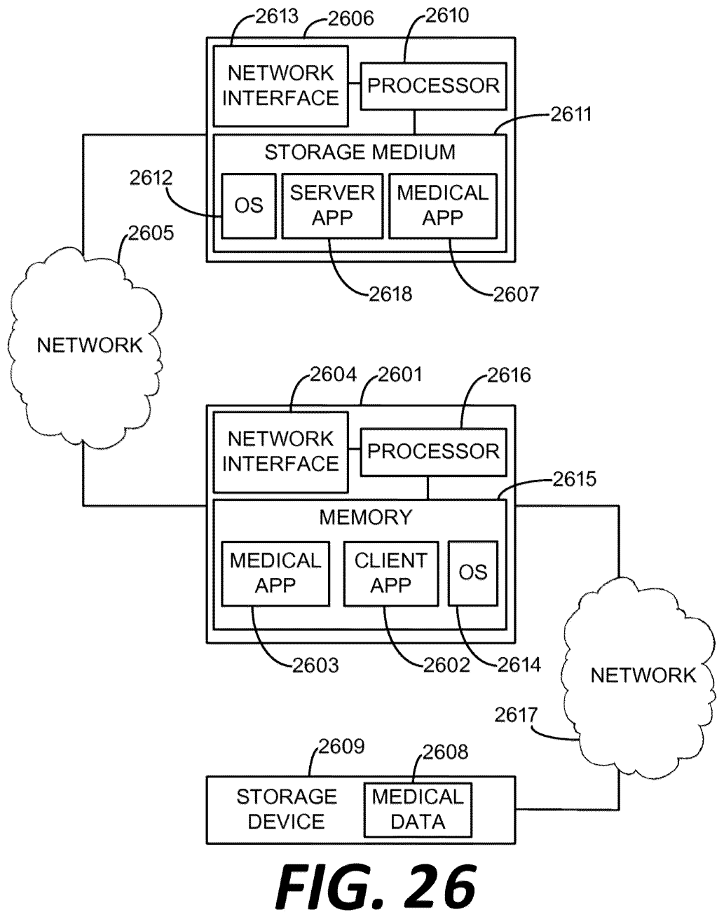

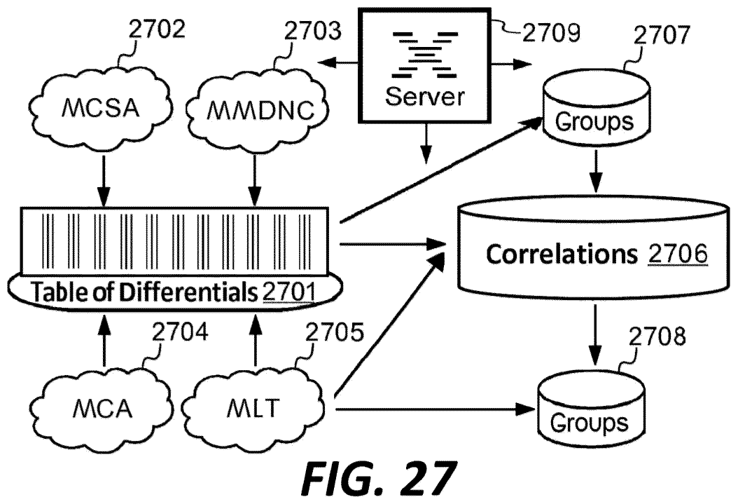

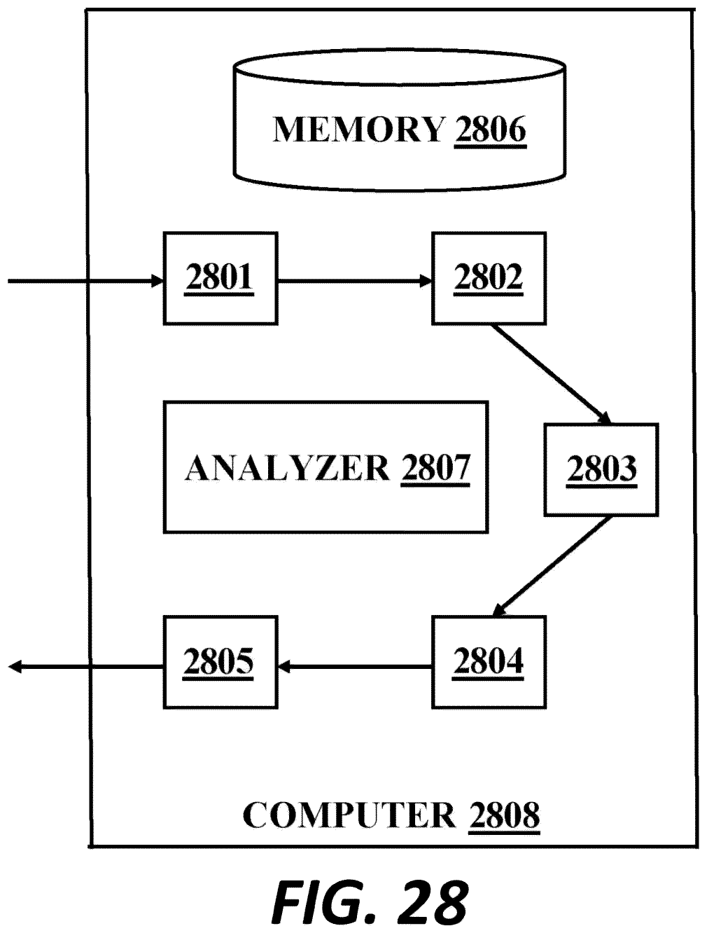

FIG. 19 is a diagram illustrating components for implementing the data transmission operation for implementing the invention. The components provide the data transmission operation between the sensor or biosensor and server. The data transmission operation 1901 in an embodiment of the present invention is carried out by using end-to-end encryption of the data by creating a key. This seems appropriate because some of the data transferred (e.g., data on the person's current illnesses or person's symptom data values) are personal data received and secure methods of data transfer between servers will provide protection against possible hacking and loss of person's personal data.

In operation, the servers 1902 and 1903 enable secure transmission of data between or on behalf of their hardware and software components through the use of data merging module (DMM) 1904 connected by network 1905. The servers 1902 and 1903 cooperate with the DMM 1904 to generate and maintain a distributed ledger 1906. The distributed ledger 1906 stores metadata associated with hardware and software components of servers 1902 and 1903. The distributed ledger 1906 implements a data structure that includes various blocks, with each block holding a batch of individual transmissions and including a timestamp indicating block inclusion in the ledger 1906.

Each server 1902 and 1903 may include a ledger management module, and a key management module. The ledger management modules manage the distributed ledger 1906. For example, the ledger management modules may propose new blocks for the distributed ledger 1906 (each proposed block containing one or more transmissions.) The ledger management module further performs operations to ensure that the network node includes an updated copy of the distributed ledger 1906. Generally, the ledger management module serves as an interface for the distributed ledger 1906. For example, the key management module may access the distributed ledger 1906 by way of the key management module. In operation, a transmission module of server 1903 may initiate a transmission with the server 1902. To execute this transmission, the transmission module of server 1903 first may query the distributed ledger 1906 to determine if a certification transmission is stored therein that would satisfy access requirements.

The distributed ledger 1906 may contain the certification transmission but not the required key. Alternatively, the distributed ledger 1906 may contain both the key and the certification transmission. Assuming only the key is not available, the server 1903 may request the server 1902 provide the required key. In response, the transmission module transmits a key request 1907 to the key management module. The key request 1907 indicates a requested transmission type, e.g., health certification (i.e., the key request 1907 is for a health credential that the transmission module requires to complete the transmission.)

The key management module provides a key 1908 in response to receiving the key request 1907. The key management module determines whether the key request 1907 is a valid request. The key management module accesses the distributed ledger 1906 to determine whether the key request 1907 satisfies one or more validation criterion. For example, the key management module may query the distributed ledger 1906 to determine whether the requested time duration, the requested number of transmissions, and/or the requested transmission type are permitted.

The key management module synthesizes the key 1908. The key 1908 may include a session key, a pair of keys (e.g., a public key and a private key.) In some examples, the pair of keys is asymmetric or a single shared key. For example, the key management module may employ a variety of symmetric-key algorithms, such as Data Encryption Standard (DES) and Advanced Encryption Standard (AES), to generate the key 1908. Alternatively, the key management module employs a variety of public-key algorithms, such as RSA, to generate the key 1908. In an aspect, the key 1908 includes a random number. In another aspect, the key 1908 is the output of a hash function, where the hash function is a hash of the names of the entities, a time of day, and/or a random number. In yet another aspect, the key 1908 includes a credential.

The key 1908 is associated with a key identifier (ID) that identifies the key, and a validity period that indicates a time duration during which the key 1908 is valid. The validity period may be equal to requested time duration. However, if the requested time duration is greater than a threshold time duration, the validity period may be limited to the threshold time duration. In an aspect, the key 1908 may be associated with a validity number that indicates the number of transmissions that can be completed with the key 1908. The validity number may be equal to a requested number of transmissions. However, if the requested number of transmissions is greater than a threshold number of transmissions, the validity number may be limited to the threshold number of transmissions. In another aspect, the key 1908 is associated with a validity type that indicates a transmission type that may be completed with the key request 1907. The validity type may be the same as a requested transmission type.

The transmission module 1909 may employ the key 1908 to synthesize the transmission data 1910. In an aspect, the transmission module 1909 signs the transmission data 1910 (e.g., a hash of the transmission data) with the key 1908. In another aspect, the transmission data 1910 includes encrypted data. The transmission module 1909 employs the key 1908 to encrypt the transmission data 1910. When encrypted, the transmission module 1909 transmits the transmission data 1910. The transmission module 1911 receives the transmission data 1910 and completes the transmission based on the transmission data 1910.

The transmission module 1911 may determine whether the transmission data 1910 is valid by, for example, determining whether the key 1908 employed to synthesize the transmission data 1910 is valid. As such, the transmission module 1911 transmits a validation request 1912 to the key management module 1913. In an aspect, the validation request 1912 includes the key 1908 (e.g., when the transmission data 1910 includes the key 1908). In another aspect, the validation request 1912 includes the key ID. In yet another aspect, the validation request 1912 includes only the transmission data 1910.

The key management module 1913 receives the validation request 1912 and determines whether the key 1908 employed to synthesize the transmission data 1910 is valid by, for example, querying the distributed ledger 1906 with the key 1908 and/or the key ID. The second key management module 1914 then transmits a validation response 1915 to the transmission module 1911. The validation response 1915 indicates a validity status of the key 1908. For example, the validation response 1915 may indicate the validity period, the validity number, and/or the validity type associated with the key 1908 are satisfied.

Based on the validation response 1915, transmission module 1911 employs the transmission data 1910 to complete the transmission. For example, the transmission module 1911 may complete the transmission if the validation response 1915 indicates that the transmission data 1910 was synthesized with a valid key (e.g., the key 1908 is valid.) In another aspect, the transmission module 1911 may access the distributed ledger 1906 to determine whether the transmission is permitted. If the distributed ledger 1906 indicates that the transmission is permitted, the second transmission module 1911 completes the transmission.

FIG. 20 illustrates components of the data merging module of FIG. 19, which is used to transmitting data between the sensor or biosensor and server. The data merging module (DMM) includes server sub-system 2001. Server sub-system 2001 in turn includes one or more CPUs 2002, network interface 2003, program interface 2004, and memory 2005. Memory 2005 is a non-transitory computer-readable memory. Memory 2005 includes server operating system (OS) 2006 and transmission module 2007. Transmission module 2007 includes machine instructions 2008, which may be loaded from non-transitory computer-readable storage medium (i.e., data store) 2009, and heuristics and metadata 2010. The CPUs 2002, network interface 2003, program interface 2004, memory 2005, and data store 2009 communicate over system bus 2011. The operating system 2006 includes procedures for handling various basic system services and for performing hardware-dependent tasks.

The transmission module 2007 manages transmissions between the sensor or biosensor and server. For example, the transmission module 2007 may transmit a key request to a network node within a cluster of network nodes that are configured to maintain a distributed ledger. The transmission module 2007 receives a key in response to transmitting the key request and synthesizes transmission data with the key. The transmission module 2007 transmits the transmission data to another entity. The transmission module 2007 receives transmission data, transmits a validation request to determine whether the key utilized to synthesize the transmission data is valid, receives a validation response, and utilizes the transmission data to complete a transmission if the validation response indicates that the key is valid. To that end, the transmission module 2007 includes machine instructions 2008, and heuristics and metadata 2010.

The memory 2005 and/or the data store 2009 also stores programs, modules, and data structures to enable a distributed ledger 2012, a ledger management module 2013, and a key management module 2014. The distributed ledger 2012 may be distributed over various network nodes. In some aspects, each network node stores a local copy of the distributed ledger 2012. The distributed ledger 2012 may store information regarding transmissions between the sensor or biosensor and server. In some aspects, the distributed ledger 2012 stores a batch of transmissions in a block. In some aspects, each block is a timestamped.

The ledger management module 2013 manages the distributed ledger 2012. For example, the ledger management module 2013 functions to ensure that the local copy of the distributed ledger 2012 is synchronized with the local copy of the distributed ledger 2012 at other network nodes. The ledger management module 2013 participates in consensus protocols associated with the distributed ledger 2012. For example, the ledger management module 2013 may propose new blocks for the distributed ledger 2012 and/or votes on block proposals received from other network nodes. To that end, the ledger management module 2013 includes machine instructions, and heuristics, and metadata.

The key management module 2014 receives a key request from an entity, determines whether the key request is valid, synthesizes a key if the key request is valid, transmits the key to the entity, and stores the key in the distributed ledger 2012. The key management module 2014 determines whether the key request is valid by determining whether one or more validation criterion stored in the distributed ledger 2012 is satisfied. For example, the key management module 2014 receives a validation request from an entity, accesses the distributed ledger 2012 to determine whether the key utilized to synthesize the transmission data is valid, and transmits a validation response that indicates the validity status of the key to the entity. To that end, the key management module 2014 includes machine instructions, heuristics, and metadata.

FIG. 21 illustrates an example of a metric of differentials that includes the values of the differentials for major SARS-CoV-2 virus strains. The left-hand column of the metric contains the results of laboratory medical tests that include tests for COVID disease (e.g., antigen test, molecular test, antibody test), and laboratory medical examinations required to obtain the values of the person's biochemical and biophysical data in relation to the symptoms of the major SARS-CoV-2 virus strains. Symptoms of the SARS-CoV-2 virus strains are variable, but in general include fever, cough, headache, fatigue, breathing difficulties, and loss of smell and taste. The severity of mutated SARS-CoV-2 virus strains varies and symptoms of the mutated SARS-CoV-2 virus strains are variable. Common symptoms include headache, loss of smell and taste, nasal congestion and a runny nose, a cough, muscle pain, a sore throat, a fever, diarrhea, and breathing difficulties. People with the same infection may have different symptoms, and their symptoms may change over time.

Laboratory medical tests listed in the left-hand column of the metric include reverse transcription polymerase chain reaction (RT-PCR) test, nucleic acid test, serological test, molecular test CRISPR, isothermal nucleic acid amplification (INAA), digital polymerase chain reaction (DPCR), microarray analysis, next-generation sequencing (NGS), antigen tests for antigen proteins, rapid diagnostic test (RDT), enzyme-linked immunosorbent assay test (ELISA), neutralization assay, chemiluminescent immunoassay (CI). Blood samples for these tests can be a whole blood, blood plasma, serum samples, isolated antibodies, blood compositions, blood substitutes. Sputum samples for these tests can be a nasal swab, nasopharyngeal swab, oropharyngeal swab, throat swab, deep airway material, saliva. Laboratory medical examinations include chest CT scans, checking for an elevated body temperature, and checking for low blood oxygen levels.

The left-hand column of the metric further contains names of symptoms and diseases, for which the person's biochemical and biophysical data is gathered by a plurality of sensors (including biosensors) for detecting respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

It should be obvious to those skilled in the art which sensors can be used for each individual symptom, and therefore, it is pointless to list all these sensors in the present invention, especially when new and enhanced sensors are continuously being introduced in medical practice (e.g., biosensors which convert a biological response into an electrical signal and combine a biological component with a physicochemical detector.) It is also obvious that as many available sensors should be used and as many laboratory medical tests, laboratory medical examinations should be run as possible to obtain maximum data. Sensors for other symptoms not mentioned above can also be used, if necessary, such as blood sugar sensors, etc.

The header of the metric contains the names of the major SARS-CoV-2 virus strains: original SARS-CoV-2 virus strain, Alpha (lineage B.1.1.7), B.1.1.7 (E484K), Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Lineage B.1.1.207, Lineage B.1.1.317, Lineage B.1.616, Lineage B.1.618, Omicron (lineage B.1.1.529). This list is not complete and can be further expanded by adding new COVID variants discovered later.

Based on the scientific medical literature, medical guidelines provide predetermined symptom threshold values that can be used to identify major SARS-CoV-2 virus strains (e.g., those listed in the header of the table.) Therefore, it is possible to calculate the actual differentials (differences) between the values of the person's biochemical and biophysical data obtained from a plurality of sensors, through laboratory medical tests, laboratory medical examinations (listed in the left-hand column of the metric), and the predetermined symptom threshold values that would help to identify the major SARS-CoV-2 virus strains. These differentials are added to the metric, so the metric contains the values of the differentials that represent differences between the values of the person's biochemical and biophysical data and predetermined symptom threshold values for the major SARS-CoV-2 virus strains.

The differentials mentioned in the present invention may be negative. For example, a healthy person's temperature is about 36.6° C., while the temperature indicative of the Delta SARS-CoV-2 virus strain is 40° C. According to conventional medical practice, if the person's temperature is 38° C., for instance, then the differential is not calculated, as the 40° C. threshold value hasn't been reached. Further diagnosis of the Delta SARS-CoV-2 virus strain is not carried out. According to the present, differentials are always calculated, and the diagnosis is carried out, even if the difference is negative (−2° C.). It is possible that differentials of other symptoms all point to the fact the person has Delta SARS-CoV-2 virus strain, while the insufficiently high temperature is due to the person's individual physiological parameters. Therefore, the data in the metric of FIG. 21 includes differentials, both positive and negative, between the values of the person's biochemical and biophysical data and predetermined symptom threshold values for the major SARS-CoV-2 virus strains.

For each of a plurality of the values of differentials within the metric of FIG. 21, which are calculated by comparing the values of data received from a person to predetermined symptom threshold values for the SARS-CoV-2 virus strains (e.g., for the first original SARS-CoV-2 virus strain and for the second mutated SARS-CoV-2 virus strain), the medical analytics platform of the present invention can identify an accurate value indicative of the likelihood that the person is experiencing the symptom of the SARS-CoV-2 virus strain (e.g., the symptom of the first original SARS-CoV-2 virus strain or the symptom of the second mutated SARS-CoV-2 virus strain) and the severity this symptom. Thereafter, it is created a metric of all differentials in which the differentials (e.g., the first and second differentials) are ordered in their values (accurate value) relative to symptoms of the SARS-CoV-2 virus strains (e.g., the symptom of the first original SARS-CoV-2 virus strain or the symptom of the second mutated SARS-CoV-2 virus strain.)

FIG. 22 illustrates examples of the metrics of differentials that includes the accurate values and complex symptoms. The accurate value indicates of the likelihood that the person is experiencing the symptom and the severity this symptom. By relying on well established, medically documented, facts and characteristics for symptoms of the SARS-CoV-2 virus strains (and discretizing measures that calibrate them), and, e.g., machine learning techniques, the accurate value (accurate values No 21-5 in 22a) can be computed for each of a plurality of the differentials (values of differential No 1-5 in 22a). Thus, accurate values for differentials that indicate the likelihood and severity (e.g., uninfected, mild, moderate, and severe) of symptom of the SARS-CoV-2 virus strain can be established.

In an aspect, machine learning techniques are used to determine accurate values for differentials that indicate the likelihood and severity of symptoms of the SARS-CoV-2 virus strains. The medical analytics platform may use a (deep or shallow) machine learning process to definite accurate values for the differentials. As additional studies are released, it is possible to update the accurate values for the differentials describing the likelihood and severity of each of the symptoms above. In another aspect, the accurate values for the differentials can be the original values of the differentials obtained by comparing the values of data received from a person representing their symptoms to predetermined symptom threshold values for the SARS-CoV-2 virus strain. In this case, the accurate values for the differentials and values of the differentials are the same.

Thus, the metrics of the differentials stores a plurality of records of values (or accurate values) of differentials, each associating a symptom with possible symptoms of the SARS-CoV-2 virus strains. It is also possible to determine complex symptoms within the plurality of the symptoms of the SARS-CoV-2 virus strains. The complex symptom is the symptom that shows the correlation of the several symptoms of the SARS-CoV-2 virus strains. For example, as shown in 22b, if symptoms comprise shortness of breath, sweating, chills, fatigue, headache, muscle pain, the possible complex symptom comprises fever that may cause the above symptoms, and the differentials lists a plurality of values of differentials (accurate values) calculated for these symptoms.

The metric of 22b also can include functions to determine a possible cause or causes of a symptom. Such functions may be in forms of an expert system or a decision tree. Examples of web-based expert system include the software owned and managed by EasyDiagnosis, a division of MatheMEDics such as www.easydiagnosis.com. It is noted that 22b is merely illustrative of a logical record. Many representations to store, retrieve, search for, or modify all or parts of the illustrated stored content are well known in the state of the art.

The metric of the differentials includes complex symptoms indicated or associated with the Delta SARS-CoV-2 virus strain. As shown in 22b, symptoms associated with the Delta SARS-CoV-2 virus strain may include shortness of breath, especially with activity, or when lying down, swelling of feet and ankles, fatigue and weakness, persistent cough or wheezing cough that may be accompanied by white or blood-tinged phlegm, rapid weight gain, irregular or rapid heartbeat, change in urine production (increase or decrease, need to urinate at night), nausea, loss of appetite, decreased alertness, and increase of respiration rate.

Fatigue and shortness of breath can be distinguished as complex symptoms that show the correlation of the noted above symptoms of the Delta SARS-CoV-2 virus strain for the indicated example. Each of these complex symptoms may be evaluated based on a well-known scale. For example, the fatigue and shortness of breath may be characterized to show the severity on a New York Heart Association scale of 0-10 (10 being very severe fatigue or shortness of breath.) Using complex symptoms within the metrics of the differentials, it may find that the person has contracted the Delta SARS-CoV-2 virus strain, if for example, feels fatigue very easily, and experiences shortness of breath.

The comprehensiveness of the information about the values (or accurate values) of the differentials and the complex symptoms within the metrics of the differentials allows to evaluate the health situation of the person and make a conclusion about a presence or absence of SARS-CoV-2 disease in a person. The record of complex symptom in 22b may include a summary of the complex symptom, possible causes of the symptom and the correlation of the several symptoms. As will be appreciated, this data can be also grouped and weighted.

FIG. 23 illustrates examples of the metrics of differentials that include the weighting coefficients. The severity of symptom of COVID disease is described by the weighting coefficient. The weighting coefficient characterizes the severity level of symptom. The weighting coefficients for symptoms of the SARS-CoV-2 virus strains may be determined, based on the latest medical documentation, indicative of higher/lower likelihoods that the person is experiencing more/less severe symptoms of the SARS-CoV-2 virus strains. The weighting coefficients for symptoms of the SARS-CoV-2 virus strains may be compared with each other. For each value (or accurate value) of differential within the metric of differentials, a higher weighting coefficient is indicative of a more severe the respective symptom of the SARS-CoV-2 virus strain. Thereafter, the values (or accurate values) of the differentials within the metric of the differentials are then ordered relative to the weighting coefficients for symptoms.

The weighting coefficient of the symptom is established by weighing the severity level of this symptom. The each symptom may be weighted based on the prevalence and correlation between each symptom and the SARS-CoV-2 virus strains, as identified in the latest medical documentation, such that a weighting coefficient of symptom below the lowest thresholds is indicative of a low likelihood that the person has contracted the SARS-CoV-2 virus strain. And vice versa, a weighting coefficient of symptom above the highest thresholds is indicative of a high likelihood that the person has contracted the SARS-CoV-2 virus strain.

23a illustrates an example of weighting coefficients for ten symptoms of the most common SARS-CoV-2 virus strains: fever, chills, cough, difficulty breathing, nasal congestion, loss of taste, sore throat, loss of smell, headache, muscle aches. As noted above, the SARS-CoV-2 symptoms within the metric of the differentials may be weighted differently when evaluating possible diseases of the SARS-CoV-2 virus strains. Thus, the weighting coefficient is determined for each above symptom that characterizes the severity level of symptom.

Thereafter the values (or accurate values) of the differentials within the metric are ordered relative to these weighting coefficients. The hierarchy of weighting symptoms of the SARS-CoV-2 virus strains in 23a is based on the utilizing a scoring/weighting range of 1-6. A score of 1 indicates the highest confidence level for a SARS-CoV-2 symptom with a score of 6 having the least reliable value for validating or verifying the symptom. It is possible to generate a weighting coefficients No 1, No 2, No 3, No 4, No 5, based on the values (or accurate values) of the differentials and the weight of symptom it has been assigned.

23b illustrates exemplary rules for weighting the SARS-CoV-2 symptoms that can be used, for example, in conjunction with the weighting coefficients identified in the metric of 23a to determine a confidence level that a SARS-CoV-2 disease is present. In order for a symptom of the SARS-CoV-2 virus strain to be deemed to have a high severity, certain combinations of weighting coefficients must exist. If the weight level based on the weighting coefficients exceeds a predetermined weight level, the condition is considered “confirmed” that the person has been diagnosed with that SARS-CoV-2 symptom.

The weight level may be determined using the weighted method using machine learning techniques or based on the presence of a certain number of weighting coefficients. For example, a symptom of the SARS-CoV-2 virus strain could be confirmed if any three of the weighting coefficients are found into the metric of the differentials. As shown in 23b, for the Delta SARS-CoV-2 virus strain, if three (or more) weighted coefficients for the Delta strain at the same time are present, the weight level is considered to be surpassed and the symptom of the Delta SARS-CoV-2 virus strain is considered to be validated or confirmed.

After performing the above steps to determine symptoms accurate values for differentials, weighting coefficients for symptoms of the SARS-CoV-2 virus strains and complex symptoms of the SARS-CoV-2 virus strains, the metric of differentials is created. The values (or accurate values) of the differentials within the metric are ordered in their values relative to symptoms of the SARS-CoV-2 virus strains (e.g., to symptoms of the first SARS-CoV-2 virus strain and symptoms of the second SARS-CoV-2 virus strain.)

The metrics of the differentials are then compared to the predetermined metric that contains known values of differentials indicating that the person has contracted SARS-CoV-2 virus strains (e.g., the first original SARS-CoV-2 virus strain or the second mutated SARS-CoV-2 virus strain.) In response to this comparison, a presence or absence of SARS-CoV-2 in a person is determined. In an aspect, the determination occurs when at least one value (accurate value) within the metric of the differentials exceeds the values within the predetermined known metric. In another aspect, the determination occurs when the majority of the values (accurate values) within the metric of the differentials exceed the values within the predetermined known metric.

Other embodiments of the present invention are possible. In an aspect, the values (or accurate values) of the differentials within the metric of differentials can be combined into multiple groups based on the differences in the values (or accurate values) of the differentials, and after that the groups of the differentials within the metric of the differentials are ordered relative to symptoms of the SARS-CoV-2 virus strains. In another aspect, the similar symptoms of the SARS-CoV-2 virus strains can be combined into a group, and after that the values (or accurate values) of the differentials within the metric of the differentials are ordered relative to multiple groups of symptoms.

Additionally, the predetermined symptom threshold values for all SARS-CoV-2 virus strains and the values within the metric of differentials may be updated so that the predetermined symptom threshold values reflect the latest understanding of symptoms of the SARS-CoV-2 virus strains. As additional studies are released, it is possible to update the accurate values for differentials, weighting coefficients for symptoms of the SARS-CoV-2 virus strains, complex symptoms of the SARS-CoV-2 virus strains.

The medical analytics platform may use a (deep or shallow) machine learning process to adjust the predetermined symptom threshold values for the SARS-CoV-2 virus strains, accurate values for differentials, weighting coefficients for symptoms of the SARS-CoV-2 virus strains, complex symptoms of the SARS-CoV-2 virus strains. Therefore, the metric of differentials is flexible that may be updated to identify additional symptoms that are found to be indicative of the SARS-CoV-2 virus strains. In the event of a future epidemic or pandemic, the disclosed metric of differentials can also be used to recognize the symptoms of a future virus.

The differentials within this metric of 23c are ordered in their values (or accurate values) relative to six most common symptoms of the SARS-CoV-2 virus strains: fever, cough, fatigue, dyspnea, anosmia, ageusia. For fever detection, a direct body temperature reading is collected and compared to the predetermined temperature threshold values for the person. Additionally, an increased heart rate is an indication of fever too. Data indicative of the heart activity of the person may be received, for example, from the heart monitor. Therefore, it is possible to detect a fever by detecting the person heart rate and comparing the detected heart rate to the predetermined heart rate threshold values for the person. The differences between the values of data received from a person (namely, body temperature, heart rate) and the predetermined symptom threshold values are the values of the differentials (3, −5, 15, 45, −12, −1) which then converted into accurate values (3, 1, 6, 8, 1, 1) indicative of the presence and severity of a fever.

Cough may be detected via acoustic engineering, e.g., using sound analysis, respiratory conditions are identifiable. Fatigue can be detected based in drop skin temperature (for example, as measured by the skin temperature thermometer), galvanic skin response data (for example, as measured by the electrodermal activity (EDA) sensor), reduced heart rate (for example, as measured by the heart monitor.) The differences between the values of data received from a person (namely, respiratory conditions, skin temperature, galvanic skin response data, reduced heart rate) and the predetermined symptom threshold values are the values of the differentials (22, −12, 3, 24, 21, 3 and 16, 21, 3, −2, 10, −5) which then converted into accurate values (9, 1, 2, 9, 9, 1 and 7, 9, 1, 1, 4, 1) indicative of the presence and severity of a cough and fatigue accordingly.

Difficulty or labored breathing (dyspnea) may be detected by identifying an increased respiratory rate (for example, as measured by the respiratory sensor) and a change in blood oxygenation (for example, as measured by the pulse oximetry sensor.) The loss of the sense of smell (anosmia) and reduced ability to smell (hyposmia) have well established diagnostic tests, such as the University of Pennsylvania Smell Identification Test (UPSIT) and “Sniffin' Sticks”, a test of nasal chemosensory performance based on pen-like odor-dispensing devices. The loss of sense of taste (ageusia) and reduced ability to taste sweet, sour, bitter, or salty substances (hypogeusia) can be detected via plurality of various sensors and biosensors. The differences between the values of data received from a person (namely, respiratory rate, change in blood oxygenation, smell test data, taste sensors data) and the predetermined symptom threshold values are the values of the differentials (35, −11, −1, 13, 17, −2 and 13, 18, −2, 11, −2, 32 and 19, 3, 43, 23, −12, 2) which then converted into accurate values (6, 1, 1, 4, 6, 1 and 4, 6, 1, 4, 1, 8 and 7, 2, 8, 7, 1, 2) indicative of the presence and severity of a dyspnea, anosmia, and ageusia accordingly.

FIG. 24 illustrates other examples of the metrics of differentials of the present invention. The differentials can be combined into multiple groups based on the differences in the values of the differentials (accurate values), and after that the groups of the differentials within the metric of the differentials are ordered relative to symptoms of the SARS-CoV-2 virus strains. 24a shows example of groups of the differentials. The values of the differentials such as −3, −11, 14, 3, 12, −5, 15, 2, −12, 6 are accordingly combined into multiple groups of the differentials [symbol] o1, [symbol] o2, [symbol] o3, [symbol] o4 that include the corresponding values of the differentials: −3, −11, −5, −12 and 14, 3, 12, 15, 2, 6 and 14, 15 and −12, 15.

In other aspects, the similar symptoms of the SARS-CoV-2 virus strains can be combined into a group, and after that the values of the differentials (accurate values) within the metric of the differentials are ordered relative to multiple groups of symptoms. 24b shows example of groups of symptoms. The SARS-CoV-2 symptoms such as fever, shortness of breath, sweating, fatigue, chills, cough, rapid heartbeat, headache, muscle pain, nausea, loss of appetite, increase of respiration rate are accordingly combined into multiple groups of symptoms [symbol] o1, [symbol] o2, [symbol] o3, [symbol] o4 that include the similar symptoms: chills, fatigue, muscle pain; cough, fever, headache, sweating; rapid heartbeat, increase of respiration rate, shortness of breath; nausea, loss of appetite.

In other aspects, the weighting coefficient for each symptom of the SARS-CoV-2 virus strain is determined. The weighting coefficient characterizes the severity level of symptom. The values of the differentials (accurate values) within the metric of the differentials are then ordered relative to the weighting coefficients. 24c shows example of weighting coefficients. The symptoms of the SARS-CoV-2 virus strains such as fever, shortness of breath, sweating, fatigue, chills, cough, rapid heartbeat, headache, muscle pain, nausea, loss of appetite, increase of respiration rate have accordingly weighting coefficients 3, 4, 6, 4, 4, 3, 4, 4, 5, 5, 2, 3 that characterize the likelihood and severity these symptoms. Thereafter the values (or accurate values) of the differentials within the metric are ordered relative to these weighting coefficients.

In other aspects, it is determined the complex symptom that shows the correlation of the several symptoms of the SARS-CoV-2 virus strains. After that the values of the differentials (accurate values) within the metric of the differentials are ordered relative to complex symptoms. 24d shows example of complex symptoms within the plurality of the symptoms: fever, shortness of breath, sweating, fatigue, chills, cough, rapid heartbeat, headache, muscle pain, nausea, loss of appetite, increase of respiration rate. The complex symptoms such as fever, shortness of breath may cause the symptoms such as rapid heartbeat, headache, muscle pain, fatigue; cough, chills, increase of respiration rate, sweating.

FIG. 25 is a diagram illustrating the medical analytics platform 2501 of the present invention. A data extraction facility 2502 can extract data from a plurality of medical data 2503 to enable the real-time collection, processing, analysis and centralized storage of medical information in a databases. Real-time, continuous data ingestion may come from various medical data 2503 which may include sensor data, biosensor data, laboratory analysis data, medical test data, test system data, person's samples data, blood oxygen data, medical examination data, ambulatory clinical data, pharmacy data, doctor's notes, medical regulations, medical instructions, medical guidelines, predetermined symptom threshold values, differential values, symptom data, metrics of the differentials, predetermined known metrics, etc.

The medical analytics platform 2501 enables ingestion and analysis of the medical data by converting the data to standardized data elements using a data normalization facility 2504 and a data processor 2505. The data processor 2505 may transform data from the various formats in which it exists. The rules database 2506 may provide rules to the data processor 2505 for analysis of medical data 2503. To do this, the rules database 2506 stores rules, instructions, guidelines, attributes, characteristics, and criteria that are used in this analysis. The data are manipulated and analyzed by the medical analytics tools. Tools may enable data mining, the machine learning techniques, etc. The analytics may be modular, such as by SARS-CoV-2 virus strain, predetermined symptom threshold value, differential, symptom, etc. The analytics may generate granular comparative data. The analytics may also enable predictive modeling.

The medical analytics platform 2501 may also comprise tools for analytic model building. For example, to build a disease model for SARS-CoV-2 virus strain, aspects of the disease that might be of interest, such as person's symptom data values, may be obtained by a plurality of sensors or a test system for an indication of a viral infectious disease. These aspects may be defined as inputs to the model in terms of rules, instructions, guidelines, attributes, characteristics, criteria, etc. These inputs may be defined in a rules database 2506 and updated periodically or as needed. Data may be analyzed according to rules of the model by the medical analytics platform 2501 to enable determining a disease in a person. The data may be stored in a flexible data warehouse, such as a raw data store 2507 and a data mart 2508. The data may be certified. Interfaces to the medical analytics platform 2501, such as a user interface 2509, report facility 2510, and other interfaces 2511, may be used to search and view data, initiate analyses, visualize data, generate reports, generate a tracking page, etc.

FIG. 26 is a diagram illustrating components for implementing the medical analytics platform of FIG. 25 using one or more medical applications, including a client computer 2601 having a client application 2602 configured to control access of one or more medical applications (e.g., a medical application 2603) to one or more client computer resources, such as a network interface 2604. The client computer 2601 can include one or more client computers, or one or more other computers configured to process medical data. The client application 2602 can be configured to communicate over a network 2605 with a server 2606 having one or more medical applications, such as the medical application 2607. Further, the client application 2602 can be configured to receive information (e.g., medical data 2608) from a storage device 2609, such as a database coupled to the client computer 2601 (e.g., using a local area network, a wide area network, etc.) In an example, the client computer 2601 can include a medical application 2603, such as at least partially received (e.g., downloaded) from the server 2606, etc. In certain examples, the client computer can include a plurality of medical applications at least partially received (e.g., downloaded) from the server 2606.

The client application 2602 can be configured to provide information to one or more medical applications stored at least partially on the client computer 2601, and to control access of one or more of the medical applications 2603 to one or more client computer resources, such as a network interface 2604. In an example, the server 2606 can include a processor 2610 (e.g., one or more processors) coupled to a storage medium 2611 (e.g., one or more hard drives, an array of hard drives, etc.) The storage medium 2611 can include a general-purpose server operating system 2612 (e.g., Linux, Microsoft Windows Server, IBM Advanced Interactive eXecutive (AIX), etc.) stored or installed thereon. In certain examples, the server operating system 2612 can manage one or more server software processes, and can include commercial or open source software, such as Apache/Tomcat, JBOSS, or IIS, or others to manage server-based processes. In an example, the server 2606 can include a physical network interface 2613 coupled to the network 2605 for communication with the client computer 2601.

The client computer 2601 can include an operating system 2614 (e.g., a general purpose operating system) stored or installed in a memory 2615 and configured to be executed on a processor 2616 coupled to the memory 2615. The client operating system 2614 can include, for example, Microsoft Windows 7, Microsoft Windows XP, Linux, Redhat, Ubuntu, Apple OS X, Google Android, Apple ITunes, or one or more other client operating systems. In certain examples, the client computer 2601 can be coupled to a storage device 2609, such as via a local area network 2617. In an example, the external storage device 2609 can include a remote server, such as via a wide area network, and can include medical data 2608 stored thereon. In an example, the external storage device 2609 can include a database of medical data, a medical imaging archive, clinical informatics storage, a laboratory/pathology system, an imaging modality, or other clinical users and information resources.

A physical network interface 2604 can be coupled to the processor 2616. In certain examples, the physical network interface 2604 can be configured to couple the client computer 2601 to the network 2605, such as for communication with server 2606. In an example, the physical network interface 2604 can couple the client computer 2601 to the network 2617, such as for communication with the external storage device 2609. In an example, the operating system 2614 can include one or more client operating system resources, such as a network interface. In an example, the operating system 2614 can include a resource configured to control access to the physical network interface 2604.

The client computer 2601 can include a client workstation running a Windows based, or other, operating system, a medical device (e.g., a magnetic resonance imaging (MRI) scanner) including a general purpose processor or memory, a mobile device (e.g., a laptop), an etc. In certain examples, the client computer 2601 can include one or more inputs (e.g., keyboard, mouse, etc.) configured to receive user requests, such as a user request for a specific medical application, a user request to process data on a medical application, account information, etc. In an example, the storage medium 2611 on the server 2606 can include one or more medical applications (e.g., a medical application 2607) stored thereon. In an example, the medical application 2607 can include a software application or executable configured to perform one or more actions on information, such as one or more items of medical data.

The client computer 2601 can include the client application 2602 stored on the memory 2615 and configured to initiate and control the execution of one or more medical applications, such as the medical application 2607. In other examples, the client application 2602 can include an executable image. In another example, the client application 2602 can include one or many binary libraries or intermediate library objects controlled by a higher-level executable image. The client application 2602 can communicate with a server application 2618 for download or control of the medical application 2607 as discussed in more detail below.

Although FIG. 26 illustrates the client computer 2601 as a single client computer, in other examples, multiple client computers can be coupled to the server 2606 over the network 2605. Accordingly, because the client computer 2601 can include multiple client computers having an instance of the client application 2602 executing thereon, multiple instances of the client application 2602 can communicate simultaneously with server application 2618. Additionally, although the server 2606 illustrated in FIG. 26 includes a single server, in certain examples, the server 2606 can include a distributed server, and can include multiple sites having synchronized databases coupled to the network 2605.

In an example, the medical application 2607 can include a virtualized application configured to be executed on a virtual platform. In an example, the medical application 2607 can include a software application that is fully installed within a container file that includes a complete run-time environment for the application. The container can include a virtualized operating system having a conventional medical application installed thereon, including an application .exe and .dll components, along with other related services including a database management system. In an example, the medical application 2607 can include a VMWare, CITRIX, or other equivalent based construct or virtual operating system.

FIG. 27 is a diagram illustrating the analysis of medical data of the present invention. The metrics of differentials of FIGS. 21-24 are grouped into the table of differentials 2701 and stored on a server 2709 or a Cloud server. Table 2701 and the metrics of FIGS. 21-24 comprises all differentials obtained for major COVID variants, which are then processed using the method of combinatorial statistical analysis 2702, the mathematical method of dense network of curves 2703, the methods of cluster analysis 2704, the machine learning techniques 2705 to detect tendencies and correlations 2706. In another aspect, the differentials within the table of differentials 2701 are combined into multiple groups of differentials 2707 based on the differences in the differentials. The differentials that were not included in the groups 2707 are not taken into account in the further analysis of the set of differentials. Based on the multiple groups of differentials 2707 detected, the set of differentials is analyzed using these methods to detect tendencies and correlations 2706 indicative of relationships between the groups of the differentials.

A set of the all detected tendencies and correlations 2706 are created. The correlations within the set are further analyzed by using the methods of cluster analysis 2704 to define the same or similar correlations and combining these correlations into a group 2708. The correlations that were not included in the groups are not taken into account in the further determination of COVID disease in a patient. Thus the multiple groups of correlations 2708 having same or similar correlations are created. All the data is stored in the databases on the server 2709. Then, these databases are analyzed by using machine learning techniques 2705 that are applied on the data saved on the databases. In another embodiment of the present invention, the databases are uploaded to the cloud server that is shared by multiple computers.

FIG. 28 is a diagram illustrating a hardware system for implementing the method of combinatorial statistical data analysis (MCSA) 2702 of FIG. 27. The system consists of 5 software components: 1) unit 2801 for lists selection (or for at least one database selection) allowing the user to enter the values and/or terms of interest in a combinatorial fashion in different lists, 2) a co-occurrence frequency retrieval unit 2802 wherein the unit extracts the co-occurrence and separately occurring statistics of the values and/or terms of interest in a combinatorial fashion from the databases, 3) a normalization unit 2803 wherein the ratio of co-occurrence statistics of the values and/or terms to the separately occurring statistics are calculated using various formulas, 4) data integration unit 2804 where the normalized data is integrated on a matrix, 5) the display unit 2805 where the data is displayed to the end-user in a graphical format.

The units 2801-2805 implemented in the central memory 2806 of a computer 2808 or on one of its storage units from a storage medium, for example, a CD-ROM, or through the transmission of a data feed. Actions on values and data are implemented through the analyzer 2807 that is loaded into the computer 2808. The aim of the method of combinatorial statistical analysis is to allow the user to enter lists of the numbers and/or terms in double or triple combinations, and compare it with each other to find specific tendencies and correlations indicative of the mathematical (statistical) or logical relationships between the values.

The method of combinatorial statistical analysis functions in the following fashion: 1) at least one database is chosen by the user, 2) the values and/or terms of interest are entered by the user in at least two lists with respect to the order of interest, 3) determination of co-occurrence as well as separately occurring frequencies for the values and/or terms of different lists in a combinatorial fashion, 4) data normalization via ratio calculation of the co-occurrence statistics to the separately occurring statistics using different ratio formulas, 5) elimination of errors and data normalization according to the normalization step, 6) graphical display of the results to the user.

The method of combinatorial statistical analysis allows the user to search for symptoms of major COVID variants, and to read and interpret the results in the following fashion: 1) the selection of the main database, 2) entrance of the values of differentials and values of patient data obtained into list 1 and list 2, 3) determination of the occurring frequencies of values in list 1 and list 2 separately on the database, 4) determination of the co-occurrence of frequencies of values in list 1 and values in list 2 in a combinatorial fashion, 5) ratio normalization of the values of frequencies of list 1 and list 2 in a combinatorial fashion, 6) error elimination with respect to results of the normalization, 7) integration of the obtained data on a matrix and displaying to the end-user using the color code. The results will show the user which symptoms of major COVID variants are probable based on the statistics of the data in table 2701.

Or, for example, we can compare and analyze the set of differentials and the set of predetermined symptom threshold values for major COVID variants to detect diseases of major COVID variants. For this we will use the method of combinatorial statistical analysis 2702 to search for COVID disease in the following fashion: 1) the selection of the main database, 2) entrance of the values of differentials and predetermined symptom threshold values into list 1 and list 2 (as below using the entrance unit), 3) determination of the occurring frequencies of values in the list 1 and list 2 separately on the database, 4) determination of the co-occurrence frequencies of values in list 1 and values in list 2 in a combinatorial fashion, 5) ratio normalization of the values of frequencies of list 1 and list 2 in a combinatorial fashion, 6) error elimination with respect to results of the normalization, 7) integration of the obtained data on a matrix and displaying to the end-user using the color code. The results will show the user which diseases of major COVID variants are probable based on the statistics of the data in table 2701.

The method of combinatorial statistical analysis 2702 is used to compare pairs of datasets in order to calculate statistics and find tendencies and correlations 2706 that are the mathematical (statistical) or logical relationships between the values among the following fifteen pairs of datasets: 1) differentials and patient's biochemical and biophysical data, 2) differentials and predetermined symptom threshold values for major COVID variants, 3) differentials and patient's individual physiological parameters, 4) differentials and diseases that accompany COVID-19, 5) differentials and additional patient data, 6) patient's biochemical and biophysical data and predetermined symptom threshold values for major COVID variants, 7) patient's biochemical and biophysical data and patient's individual physiological parameters, 8) patient's biochemical and biophysical data and diseases that accompany COVID-19, 9) patient's biochemical and biophysical data and additional patient data, 10) predetermined symptom threshold values for major COVID variants and patient's individual physiological parameters, 11) predetermined symptom threshold values for major COVID variants and diseases that accompany COVID-19, 12) predetermined symptom threshold values for major COVID variants and additional patient data, 13) patient's individual physiological parameters and diseases that accompany COVID-19, 14) patient's individual physiological parameters and additional patient data, 15) diseases that accompany COVID-19 and additional patient data.

In order to detect tendencies and correlations 2706 in a large array of data, e.g., comparing three or more datasets and finding tendencies and correlations there, the mathematical method of dense network of curves 2703 is used, for instance, to detect correlations between differentials, patient's biochemical and biophysical data, patient's individual physiological parameters, patient's diseases that accompany COVID-19, and additional patient data. The mathematical method of dense network of curves allows for a superior level of analysis of the aforementioned data, both qualitatively and quantitatively.

FIG. 29 is a diagram illustrating a hardware system for implementing the mathematical (regression data analysis) method of dense network of curves (MMDNC) 2703 of FIG. 27. The system of FIG. 29 includes the computer 2901, the chronological set of numerical values 2902, the central memory 2903, the unit of data entry 2904, the analyzer 2905, the display unit 2906. The chronological set of values 2902 is entered into the unit of data entry 2904 stored in the central memory 2903 of a computer 2901 or on one of its storage units from a storage medium, for example, a CD-ROM, or through the transmission of a data feed. The numerical values of the chronological set 2902 are used in the system in order to construct a dense network of curves constituting the topological structure of the set. Operations on the numerical values of the chronological set 2902 according to the mathematical formula (that be given below) are implemented through the analyzer 2905 that is loaded into the computer 2901.

The system is used to construct a dense network of curves constructed mathematically from numerical data of the chronological set 2902 (e.g., the values of the patient's biochemical and biophysical data or the values of the differentials) and defined by a primary parameter (the number of data points used) and a secondary parameter (the scale parameter). The secondary parameter (the scale parameter) can be the interval of time separating two consecutive data points, for example, minutes, hours, or days, since the onset of the illness or the patient was in quarantine. Other types of intervals can also be used. For differentials, for example, the scale can be expressed in terms of the number of same values of differentials to one patient or the number of same values of differentials to one major COVID variant. Or, for example, a dense network of curves can be constructed mathematically from the values of differentials of the chronological set 2902 and defined by a primary parameter (the number of patients with ischemic heart disease and tuberculosis) and a secondary parameter (the duration of the patient's illness or having immunity).

The system can use any of the following regressions: 1) regression of order zero, otherwise known as average, 2) first order regression, otherwise known as linear regression, 3) second order regression, otherwise known as quadratic regression, 4) regression of order greater than 2. The curves of this network belong to one of the following categories: 1) moving regression (MR) of degree zero, known as the moving average (MA), 2) MR of the first degree, known as the moving linear regression (MLR), 3) MR of the second degree, which we will call the moving quadratic regression (MQR), 4) MR of the kth degree, which we will call the moving k regression (MKR).

The present invention is based on the utilization of a dense network of MRs corresponding to a large set of values of the primary parameter, chosen according to defined criteria because in this case-characteristic figures appear strikingly on the monitor of a computer 2901. The network described in what follows is composed of MLRs. It is on the presence of these characteristic figures within the dense network that rests the ability to the analysis of the data and obtain precise and reliable information. The method can also use adjusted data, for example, averaged or weighted data.

The necessary conditions under which the characteristic figures appear in the network are the following: 1) the network must contain a large number of MLRs, greater than about 50, 2) the set of the values of the primary parameter must extend over a sufficiently large range, 3) the distribution of the values of the primary parameter must be such that the corresponding network has a uniform density on average. In practice, criterion 3 is satisfied when the values of the primary parameter constituting the set grow slowly and uniformly. Furthermore, if wished, one can slightly modify the density, for example, by making the network denser for smaller values of the primary parameter.

The algebraic formula used in the present invention is:

$$nk = n 1 + ( k - 1 ) a + k ( k - 1 ) N ( N - 1 ) [ nN - n 1 - ( N - 1 ) a ]$$

$$where :$$

$$k = \{ 1 , … N \} ,$$

N is the number of curves in the network,
n₁ is the first term of the set,
n_{N} is the N^{th} term of the set,
a is the interval between n₁ and n₂.

Taking N=100, n₁=8, n_{N}=1502, and a=8 as an example, one obtains for the primary parameter the following set of values: {8, 16, 24, 33, 41, 50, 59, 68, . . . , 1351, 1372, 1393, 1415, 1436, 1458, 1480, 1502}. This set of values generates a network of 100 MLRs which has a uniform density on average and extends over a large range. The characteristic figures seen on the monitor of the computer belong to one of the following two types: 1) cord, and 2) envelopes. A cord is a pronounced condensation of curves that stands out from a less dense background of curves of the network. An envelope outlines the boundary of a group of curves of the network. A characteristic figure attracts or repels the representative curve of the data, depending on its type, its shape and its relative position to the representative curve of the data. The more marked the characteristic figure, the stronger the attraction or the repulsion.

The analysis of the data requires the examination of the ensemble of the cords and envelopes and the representative curve of the data up to a given moment, over a sufficiently large interval of consecutive data points. An interval is considered sufficiently large when it contains a peripheral characteristic figure at the top of the network exhibiting a convex upward turning point and another one at the bottom exhibiting a convex downward turning point. The ensemble of the cords and envelopes and the representative curve of the data observed over a sufficiently large interval are referred to as a spatial configuration. Qualitative and quantitative indications are obtained from a spatial configuration by determining which characteristic figures specifically attract and which characteristic figures specifically repel the representative curve of the data, and this is achieved through the examination of numerous and varied spatial configurations.

FIG. 30 illustrates a graphical example of using the mathematical method of dense network of curves 2703 of FIG. 27 in which characteristic figures and spatial configurations appear. In FIG. 30, which represents a network of one hundred and fifty curves based on linear regressions calculated by the formula described above, one can see characteristic figures containing cords 1a, 1b, 1c, envelopes 2a, 2b, 2c, mixed figures (which is both a cord and an envelope) 3a, 3b and the representative curve of the set of values 4, in the form of a continuous curve.

The network contains on the upper part a peripheral characteristic figure presenting a maximum 5 and on the lower part a peripheral characteristic figure presenting a minimum 6. A characteristic figure will attract-repulse the representative curve of the chronological set of values according to its type, its shape, and its position in relation to the representative curve. For example, it is, at abscissa x0, the “attractive-repulsive” effect of the characteristic figures on the representative curve of the chronological set of values, without figure-crossing 7a, 7d, 7e, 7h, 7i and with figure-crossing 7b, 7c, 7f, 7g.

As shown in FIG. 27 by using the method of combinatorial statistical data analysis 2702, the mathematical (regression data analysis) method of dense network of curves 2703, the methods of cluster analysis 2704, it is possible to analyze all data stored in the databases on the server 2709 in the system of the present invention in order to find tendencies and correlations 2706. Also, the method of combinatorial statistical analysis 2702, the mathematical method of dense network of curves 2703, the methods of cluster analysis 2704 can be employed independently as machine learning techniques using the aforementioned databases on the server 2709 to predict new variants of COVID-19, or create novel learning models for detecting new COVID variants.

The methods 2702-2705 disclosed above are used to calculate statistics, and detect and analyze tendencies and correlations 2706 that are the mathematical or logical relationships between the values in the following data: 1) biochemical and biophysical data obtained from sensors and biosensors (for detecting cough, sputum, shortness of breath, fever, anosmia, ageusia, nasal congestion, runny nose, sore throat, muscle pain, joint pain, headache, fatigue, abdominal pain, vomiting, diarrhea, diabetes, lung diseases, cardiovascular diseases, ischemia, hypertension), 2) biochemical and biophysical data obtained from laboratory medical examinations (chest CT scans, checking for elevated body temperature, checking for low blood oxygen level), 3) biochemical and biophysical data obtained from laboratory medical tests (the reverse transcription polymerase chain reaction test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay), 4) patient's individual data (whether they have tuberculosis, diabetes, pregnancy, severe immunosuppression, lymphoma, oncological diseases, ulcers, ischemic heart diseases, cardiovascular pathologies, nervous diseases, as well as their sex, age, height, weight, ethnicity, area of living, quarantine stay length, etc.)

The method of combinatorial statistical analysis 2702, the mathematical method of dense network of curves 2703, the methods of cluster analysis 2704, the machine learning techniques 2705 can be used to detect tendencies and correlations 2706 in this data in order to diagnose the patient's viral disease, identify the major COVID variant that is closely related to the patient's disease, find differences between diseases caused by the major COVID variants, update and revise predetermined symptom threshold values for the major COVID variants, predict the individual traits of the course of the patient's viral disease, detect individual traits of the patient's diseases that accompany COVID-19, detect the post-COVID syndrome of the patient. Detected tendencies and correlations that are the mathematical or logical relationships between the values can then be used to create machine learning techniques for detecting new COVID variants.

The method of combinatorial statistical analysis 2702 compares and analyze the values of biochemical and biophysical data obtained from sensors and through laboratory medical tests, laboratory medical examinations with predetermined symptom threshold values indicating to any of the major COVID variants: Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc. The data is included in lists 1 and 2 respectively, and statistics are calculated using the combinatorial approach.

In cases when a sensor reading or test result exceeds the predetermined symptom threshold values, the resulting differentials will be positive. All major COVID variants are ranked according to the total number of positive differentials they have, from the higher total to the lower total. The top-ranked COVID variant, which has more positive differentials than other variants, will be the patient's diagnosed viral disease of major COVID variant (the corresponding diagnosis is provided), and the next COVID variant will be the closely related major COVID variant, which is the closest to the patient's diagnosed viral disease (the diagnosed major COVID variant that has infected the patient.)

When the mathematical method of dense network of curves 2703 is used to analyze all data stored in the databases on the server 2709 in the system of the present invention and interpret results, the primary parameter includes both values of differentials and predetermined symptom threshold values for major COVID variants, and the secondary parameter includes the values of the patient's biochemical and biophysical data, which are periodically updated. The resulting cords and envelopes for the representative curve of the data will show the diagnosed major COVID variant (i.e., the data forming the cord or envelope are located closer to the representative curve of the data) and the closely related major COVID variant (i.e., the data forming the characteristic figure are located further away from the representative curve of the data.)

The viral disease of a major COVID variant and the closely related major COVID variant can be detected separately using the method of combinatorial statistical analysis 2702, the mathematical method of dense network of curves 2703, the methods of cluster analysis 2704. Then all detected major COVID variants can be summed up and ranked using both methods, and the top two COVID variants may be interpreted as the patient's viral disease of major COVID variant, and as the major COVID variant that is closely related to the patient's disease respectively.

Using the method of combinatorial statistical analysis 2702 to calculate statistics for the values of differentials for all major COVID variants, and the values of differentials for the original COVID-19 virus strain, which are included in lists 1 and 2 respectively, the probability of the patient being infected by a major COVID variant is calculated, and the differences between this viral disease and the original COVID-19 virus strain are determined, in case the values of differentials of the diagnosed disease do not match the values of differentials or predetermined symptom threshold values for the original COVID-19 virus strain.

In the same way, individual traits of the viral disease course are determined, wherein all values of differentials and values of patient's biochemical and biophysical data obtained from sensors and through laboratory medical tests, laboratory medical examinations are included in lists 1 and 2 respectively, and the biochemical and biophysical data are periodically updated. By calculating statistics for differentials and biochemical and biophysical data obtained over the course of the patient's viral disease using the combinatorial method, it is possible to see the progress of the viral disease. For example, if the values of differentials increase over time, then the disease is intensifying. Conversely, if the values of differentials decrease over time, then the disease is abating.

The resulting statistical tendencies and correlations 2706 may be uploaded into the method of combinatorial statistical analysis 2702 again and compared, for example, with the patient's individual physiological parameters and additional patient data. Matches with certain patient's individual physiological parameters found therein might show individual traits of the patient's disease course. If the resulting tendencies and correlations 2706 are compared with the diseases that accompany COVID-19, then the combinatorial method might show individual traits of the patient's diseases that accompany COVID-19, as well as their possible post-COVID syndrome.

Also, in order to detect tendencies and correlations 2706, the mathematical method of dense network of curves 2703 is used, wherein the primary parameter includes both values of differentials and predetermined symptom threshold values for major COVID variants, and the secondary parameter includes statistical data of major COVID variants by sex, age, and region. The resulting spatial configuration represented by cords and envelopes and applied to a representative curve of the data will show the differences between major COVID variants, in case some characteristic figures will be detected that can be compared using the primary parameter and the secondary parameter data.

So, if the primary parameter includes both values of differentials and predetermined symptom threshold values for major COVID variants, the secondary parameter includes statistical data of major COVID variants by sex, age, and region, then the characteristic figures in the spatial configuration might point out predetermined symptom threshold values to be updated, in case the characteristic figures show much difference in their secondary parameters and/or their positions in relation to the representative curve of the data. When the mathematical method of dense network of curves 2703 is used to determine the post-COVID syndrome, the primary parameter includes both the data about the patient's diseases that accompany COVID-19 and the patient's individual physiological parameters, and the secondary parameter includes both the values of differentials and the values of patient's biochemical and biophysical data. The characteristic figures in the spatial configuration might point out tendencies and correlations between the data that are used to generate the characteristic figures. The values of the primary parameter for the characteristic figures will show the corresponding accompanying diseases, which, together with the patient's viral disease diagnosed as a major COVID variant, can be used to determine a possible post-COVID syndrome.

Alternatively, the primary parameter may include both the data about the patient's diseases that accompany COVID-19 and the patient's individual physiological parameters, and the secondary parameter includes the values of biochemical and biophysical data that are updated periodically. The characteristic figures in the spatial configuration might point out tendencies and correlations 2706 that may be used to determine the individual traits of the course of the patient's diseases that accompany COVID-19, based on the primary parameter with the characteristic figure, in case the data of the secondary parameter for the same characteristic figure change faster (meaning that the accompanying disease is intensifying) or slower (meaning that the accompanying disease is abating). Also, both the primary parameter and the secondary parameter may include the data from any of the detected tendencies and correlations 2706, which will be analyzed and interpreted again using the mathematical method of dense network of curves 2703.

It should be obvious to those skilled in the art that the data stored in the databases on the server 2709 can be analyzed using different mathematical methods. For example, the cluster analysis 2704 can be used for this. Cluster analysis is the task of grouping a set of objects in such a way that objects in the same group (called a cluster) are more similar to each other than to those in other groups (clusters). The cluster analysis 2704 can use the differences in the values of the all data stored in the databases on the server 2709 in the system of the present invention to define multiple groups of the values and to find tendencies and correlations 2706 in each group. For example, the cluster analysis uses the differences in the differentials within the table 2701 to define multiple groups of the differentials 2707 and to find tendencies and correlations 2706 in each group of the differentials.

FIG. 31 is a flowchart illustrating the steps of the algorithm for detecting COVID variants according to a first embodiment of the present invention. The system of the present invention for detecting new COVID variants includes a group of servers (a central server, a medical server, an analytical server, a machine learning server, a certification server), on which operations are performed according to the algorithm, comprising the following steps. At least one patient's biochemical and biophysical data is obtained in step 3101 from the sensors (including biosensors with a mixed biological component) and through the medical tests that can be tests for COVID disease (e.g., antigen test, molecular test, antibody test). The biosensors of the present invention utilizing two different biological components have the first and second biological components (e.g., proteins (or protein structures), aptamers, lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye, etc.) that are separated by an internal membrane and coupled to a physicochemical detector or amplifier within the biosensor.

The values of the patient's biochemical and biophysical data are compared to predetermined symptom threshold values for the original COVID-19 virus strain (the first SARS-CoV-2 virus strain) in step 3102 to calculate differentials (positive or negative). The values of the patient's biochemical and biophysical data are compared to predetermined symptom threshold values for at least one major COVID variant (the second SARS-CoV-2 virus strain) in step 3103 to calculate differentials (positive or negative). In another embodiment of the present invention, in step 3104, the major COVID variant (the second SARS-CoV-2 virus strain) that is closely related to the patient's disease is detected based on a correspondence of its symptoms to the differentials detected in step 3102 by comparing the values of the patient's biochemical and biophysical data to predetermined symptom threshold values for the original COVID-19 virus strain (the first SARS-CoV-2 virus strain). The values of the patient's biochemical and biophysical data are compared to predetermined symptom threshold values for the closely related major COVID variant (the second SARS-CoV-2 virus strain) in step 3105 to calculate differentials (positive or negative).

In an aspect, the closely related major COVID variant (the second SARS-CoV-2 virus strain) is detected such that its symptoms correspond to a majority of the differential detected in step 3102 by comparing the values of the patient's biochemical and biophysical data to predetermined symptom threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected in step 3102 by comparing the values of the patient's biochemical and biophysical data to predetermined symptom threshold values for the original COVID-19 virus strain (the first SARS-CoV-2 virus strain).

The set of all differentials is created in step 3106. In step 3107, the differentials are combined within the set of differentials into multiple groups based on the differences in the differentials. The differentials not included in the groups are not taken into account in the further analysis of the set of differentials. Based on the multiple groups of differentials, the set of differentials are analyzed in step 3108 to detect correlations (tendencies) indicative of relationships between the groups of the differentials. Based on the detected correlations (tendencies), a patient's viral disease is diagnosed in step 3109 in the event that at least one detected correlation (tendency) indicates that the patient is likely to have contracted the COVID disease. The determining in step 3110 that the patient has the viral disease is based on a confirmation of its symptoms with the values of the patient's biochemical and biophysical data obtained from the sensors and through the medical tests in step 3101. In response to a determination that the patient has or has not contracted the disease, a diagnosis indicating the presence or absence of a disease is generated in step 3111. The diagnosis can be a test result indicating the presence or absence of COVID disease in a patient.

Based on the received diagnosis, in step 3112, the system generates a patient's health certificate, which includes the patient's disease, the test result for the patient that indicates the presence or absence of COVID disease, the viral risk score, the difference between the disease and the original COVID-19 virus, the probability of the patient being infected by a new COVID variant, the major COVID variant that is closely related to the disease, patient's diseases that accompany COVID-19, the disease statistics by criterion (e.g., sex, age, region), the projected post-COVID syndrome for the patient. In an aspect, the patient's health certificate is storied to a database on a computer for further outputting or displaying. In another aspect, the patient's health certificate is transmitted to a mobile electronic device using end-to-end encryption. In yet another aspect, the patient's health certificate is loaded to a Cloud server shared by multiple computers.

FIG. 32 is a flowchart illustrating the steps of the algorithm for detecting COVID variants according to a second embodiment of the present invention. The patient's biochemical and biophysical data is obtained from a variety of sensors in step 3201. In another aspect, the medical institution runs laboratory medical tests for COVID disease (e.g., antigen test, molecular test, antibody test), laboratory medical examinations for COVID disease and so obtains biochemical and biophysical data in step 3201. The sensors may include a smartphone, a pulse oximeter, a body temperature thermometer, etc. The sensors also may include biosensors of the present invention utilizing two different biological components with the first and second biological components separated by an internal membrane within the biosensor. The data is transferred by the sensor using a secure encoded channel. The process may be performed by the server or central processing unit in conjunction with the user device (e.g., running a software program provided by the server or central processing unit). The sensor data may provide direct evidence the user is experiencing one of the symptoms.

Then, the values of the patient's biochemical and biophysical data obtained is compared to predetermined symptom threshold values for the original COVID-19 virus strain (the first SARS-CoV-2 virus strain) in step 3202, e.g., the values of the patient's biochemical and biophysical data obtained through laboratory medical tests are compared to positive and negative predetermined IGM and IGG antibody values indices used as references for determination viral diseases, particularly, COVID disease. Based on this comparison, the probability of the patient having this viral disease is assessed, and it is concluded whether the patient is infected with COVID disease or not in step 3203. Therefore, the patient's viral disease is identified.

However, the process does not stop here and returns to steps 3201, in which more patient's biochemical and biophysical data is obtained from sensors and through laboratory medical tests, laboratory medical examinations. This updated patient data is again compared with predetermined symptom threshold values in step 3202. Additionally, the set of the patient's individual physiological parameters, such as age, gender, blood type, blood pressure, blood sugar, immunity, vaccination history, etc., is generated. This set may also include diseases that accompany COVID-19, e.g., tuberculosis, diabetes, severe immunosuppression, lymphoma, oncological diseases, ulcers, cardiovascular pathologies, nervous diseases, etc.

Thus, the values of the patient's biochemical and biophysical data obtained from sensors and through laboratory medical tests, laboratory medical examinations are compared to predetermined symptom threshold values for the original COVID-19 virus strain (the first SARS-CoV-2 virus strain) in step 3202. Differences are calculated, and resulting differentials, both positive and negative, are recorded. The differentials are negative when values of the patient's biochemical and biophysical data obtained do not exceed the predetermined symptom threshold values, and positive when values of the patient's biochemical and biophysical data obtained exceed the predetermined symptom threshold values.

If a resulting differential is positive, it means the value of the patient's biochemical and biophysical data exceeds the predetermined symptom threshold value for the original COVID-19 virus strain (the first SARS-CoV-2 virus strain), and the diagnostic follows the algorithm disclosed herein. If a resulting differential is negative, it means the patient's biochemical and biophysical data is below the threshold, and the process returns to step 3201, in which additional data is obtained from using sensors and/or using new laboratory medical tests (and/or laboratory medical examinations), which are run by the medical institution.

In an embodiment of the present invention, in step 3204, the differentials (positive or negative) obtained in step 3202 by comparing the values of the patient's biochemical and biophysical data to predetermined symptom threshold values for the original COVID-19 virus strain (the first SARS-CoV-2 virus strain) are used to identify a major COVID variant (the second SARS-CoV-2 virus strain) that is closely related to the patient's viral disease diagnosed in step 3203. The closely related major COVID variant (the second SARS-CoV-2 virus strain) is detected based on a correspondence of its symptoms to the differentials calculated in step 3202.

In an aspect, the closely related major COVID variant (the second SARS-CoV-2 virus strain) is detected such that its symptoms correspond to a majority of the differential. In another aspect, the closely related major COVID variant (the second SARS-CoV-2 virus strain) is detected such that its symptoms correspond to a minority of the differential. Then, the values of the patient's biochemical and biophysical data is compared to predetermined symptom threshold values for the closely related major COVID variant (the second SARS-CoV-2 virus strain) in order to calculate differentials (positive or negative) in step 3205.

In another embodiment of the present invention, when obtaining differentials in step 3202 by comparing the values of the patient's biochemical and biophysical data to predetermined symptom threshold values for the original COVID-19 virus strain (the first SARS-CoV-2 virus strain), the patient data obtained is additionally compared to predetermined symptom threshold values for all major COVID variants in step 3206 to calculate differentials (positive or negative) for all major COVID variants. All differentials are grouped in step 3207 into the set of differentials (for example, differentials can be combined into the table of differentials for further analysis). In an embodiment of the present invention, in step 3208, this set of differentials is complemented by the set of the patient's biochemical and biophysical data obtained. In another embodiment of the present invention, in step 3209, this set of differentials is complemented by the set of patient's individual physiological parameters, including diseases that accompany COVID-19.

The complemented set of differentials is then analyzed in step 3210 using statistical methods (e.g., the method of combinatorial statistical analysis, the methods of cluster analysis), mathematical methods (e.g., the mathematical method of dense network of curves) to detect correlations (tendencies) within he set. The correlations (tendencies) indicative of relationships between within the values of the complemented set of differentials. Based on the detected correlations (tendencies) in step 3210 that are the mathematical or logical relationships between the values, a viral disease is diagnosed in step 3211, wherein the following processes are involved: diagnosing the patient's viral disease and getting test result indicating the presence or absence of COVID disease, detecting a major COVID variant (the second SARS-CoV-2 virus strain) that is closely related to the patient's viral disease diagnosed (the process returns to steps 3202 and 3204), keeping statistics of the viral disease course depending on the set of differentials (the process returns to steps 3203 and 3207), predicting the disease course based on the set of differentials and patient's individual physiological parameters (the process returns to steps 3207 and 3209), determining individual traits of the course of the patient's diseases that accompany COVID-19 (the process returns to steps 3203 and 3209), predicting the post-COVID syndrome and its idiosyncrasies (the process returns to steps 3209 and 3211.)

Based on the resulting diagnosis, machine learning techniques are determined in step 3212 for the set of differentials and the set of patient's individual physiological parameters, including diseases that accompany COVID-19, in order to determine individual traits of the disease course (the process can be return to steps 3203 and 3211). In another embodiment of the present invention, machine learning techniques are used to update and/or adjust predetermined symptom threshold values for all major COVID variants (the process returns to steps 3202 and 3206). In another embodiment of the present invention, machine learning techniques are used to adjust predetermined symptom threshold values for all major COVID variants, considering the patient's individual physiological parameters that include diseases that accompany COVID-19 (the process returns to steps 3206 and 3209.)

Using machine learning techniques the post-COVID syndrome that can be expected for the identified major COVID variant, taking into account the patient's individual physiological parameters, including diseases that accompany COVID-19, is predicted in step 3213. For example, the post-COVID syndrome for the Delta variant often involved increased fatigue, long-term nasopharyngeal inflammation, voice changes, impaired memory, cognitive failures (slower reaction, inability to operate properly, etc.), impaired hearing, intestinal disorders, lung and heart lesions, increased susceptibility to other infections.

A system generates a diagnosis in step 3214. The diagnosis is displayed on the smartphone screen in real-time, showing the risk of the patient being infected by a major COVID variant. In step 3215, the diagnosis is represented as a patient's health certificate, in which the patient's viral disease is given. The patient's health certificate comprises the representation of the biometric sample of the patient. The biometric sample is one or more of a thumbprint set recorded from the patient, a retina scan recorded from the patient, and a DNA sample obtained from the patient and analyzed, etc.

The patient's health certificate provides the patient's detected viral disease, the test result for the patient that indicates the presence or absence of COVID disease, the viral risk score, the probability of the patient being infected by a COVID variant, individual differences between the patient's viral disease diagnosed and the original COVID-19 virus strain (the first SARS-CoV-2 virus strain), the major COVID variant (the second SARS-CoV-2 virus strain) that is closely related to the patient's disease, possible individual traits of the patient's disease course based on the patient's individual physiological parameters, general statistics of the course of the patient's disease depending on their sex, age, area of living, etc., the projected post-COVID syndrome.

The certification server can be communicatively coupled to an internal API for transmission of health certificates to electronic medical records and human resources records in medical institutions. External APIs can be communicatively coupled to the certification server to query the health certificates associated with the patient. For external APIs, the system can output the necessary information based on the type of entity requesting the information, for example, to access a specific venue, which is any public place with a large number of people, where permission to enter is required and where the chance of a spread of a viral infection is greater.

The generated patient's health certificate can be tied to the person's ID and used for various digital identifications of the patient. The person's ID number for each respective patient can be electronically tied to their corresponding health certificate, and then the person ID can be used as a unique electronic element or identifier to access with subsequent queries for a health certificate of the patient. The system can output the necessary information based on the type of entity requesting the information, and can output to a requestor an indication the patient has or does not have a viral disease COVID variant. For this, the patient's health certificate includes a code (e.g., a QR code) capable of being scanned to display the health certificate on user interfaces or an electronic device.

In some embodiments of the present invention, step 3209 has an additional step, in which, based on the analysis of the set of differentials and the set of the patient's individual physiological parameters, a major COVID variant (the second SARS-CoV-2 virus strain) is identified, which is closely related to the patient's viral disease that has been diagnosed in step 3203. Then, the process returns to step 3205, in which the biochemical and biophysical data is compared with the predetermined symptom threshold values for the related major COVID variant (the second SARS-CoV-2 virus strain) to calculate differentials. Then, a new set of differentials is generated and analyzed using statistical methods, mathematical methods, machine learning techniques to detect correlations (tendencies) that are the mathematical or logical relationships between the values within the set. Based on the correlations (tendencies), the viral disease is diagnosed again, but with higher precision.

In some embodiments of the present invention, according to the algorithm, databases with patient's biochemical and biophysical data, patient's individual physiological parameters, diseases that accompany COVID-19, additional patient data, predetermined symptom threshold values for the original COVID-19 virus (the first SARS-CoV-2 virus strain), and predetermined symptom threshold values for at least one major COVID variant (the second SARS-CoV-2 virus strain) are generated. Then, these databases are analyzed using statistical methods, mathematical methods, machine learning techniques that are applied on all data saved on the databases. Based on the results of the analysis and detected correlations (tendencies), the viral disease is diagnosed again, but with higher accuracy.

In some embodiments of the present invention, the tests for COVID disease are conducted and results are awaited. According to the algorithm, the system can determine the IGG antibody index for the patient in step 3201, the system can determine any prior conditions associated with the patient in step 3203, then again the system can determine an IGM antibody index for the patient in step 3201, and the system determines the patient's individual physiological parameters in step 3209. The data can include manual testing and/or automated testing results, both in real-time and previously performed tests.

Steps 3202 and 3206 can incorporate medical guidelines associated with predetermined symptom threshold values for all major COVID variants to determine whether the IGG index or the IGM index, respectively, are at levels below or above the predetermined symptom threshold value. If tested positive, differentials are automatically determined for all major COVID variants. Also, again the same procedure is followed for the patient's individual physiological parameters. Whenever a patient tests positive, the system will list the data of differentials and data of the patient's individual physiological parameters.

The IGG index, IGM index, differentials, and the patient's individual physiological parameters can be used to generate a risk score or level. The risk score or level can be updated in a real-time or substantially real-time manner as additional test data is obtained and/or as medical guidelines are updated. The information is saved in step 3212 on the database and that data is analyzed using machine learning techniques. The reports and graphs from machine learning computers are stored on a cloud server for conclusions and suggestions. While a preferred embodiment has been set forth above, those skilled in the art will readily appreciate that other embodiments can be realized within the flowchart of the algorithm.

FIG. 33 is a flowchart illustrating the steps of the algorithm for detecting COVID variants according to a third embodiment of the invention. The symptom data values from at least one person are obtained in step 3301 from the sensors, biosensors of the present invention utilizing two different biological components, and through the medical tests (e.g., antigen test, molecular test, antibody test), medical examinations. The person's symptom data values are compared to predetermined symptom threshold values for the first original SARS-CoV-2 virus strain in step 3302 to calculate first differentials (positive or negative).

In step 3303, the second mutated SARS-CoV-2 virus strain is detected based on a correspondence of its symptoms to the first differentials detected in step 3302 by comparing the person's symptom data values to predetermined symptom threshold values for the first original SARS-CoV-2 virus strain. In an aspect, the second SARS-CoV-2 virus strain is detected such that its symptoms correspond to a majority of the first differentials. In another aspect, the second SARS-CoV-2 virus strain is detected such that its symptoms correspond to a minority of the first differentials. The person's symptom data values are compared to predetermined symptom threshold values for the second SARS-CoV-2 virus strain in step 3304 to calculate second differentials (positive or negative).

In step 3305, the third mutated SARS-CoV-2 virus strain is detected based on a correspondence of its symptoms to the second differentials detected in step 3304 by comparing the person's symptom data values to predetermined symptom threshold values for the second mutated SARS-CoV-2 virus strain. In an aspect, the third SARS-CoV-2 virus strain is detected such that its symptoms correspond to a majority of the second differential. In another aspect, the third SARS-CoV-2 virus strain is detected such that its symptoms correspond to a minority of the second differential. The person's symptom data values are compared to predetermined symptom threshold values for the third SARS-CoV-2 virus strain in step 3306 to calculate third differentials (positive or negative).

The metric of differentials is created in step 3307. The first, second and third differentials within metric of differentials are ordered in their values relative to symptoms of the first SARS-CoV-2 virus strain, symptoms of the second SARS-CoV-2 virus strain and symptoms of the third SARS-CoV-2 virus strain. In step 3308, the metric of differentials are compared to the known predetermined metric that contains known values of differentials indicating that the person has contracted the first SARS-CoV-2 virus strain, the second SARS-CoV-2 virus strain or the third SARS-CoV-2 virus strain.

Based on the comparison, a presence or absence of SARS-CoV-2 virus strain in a person is determined in step 3309. In an aspect, the determination that the person has contracted the SARS-CoV-2 virus strain occurs when at least one value within the metric exceeds the values within the predetermined known metric. In another aspect, the determination that the person has contracted the SARS-CoV-2 virus strain occurs when the majority of the values within the metric exceed the values within the predetermined known metric. In response to a determination that the person has or has not contracted the SARS-CoV-2 virus strain, a result (e.g., a person's health certificate) indicating the presence or absence of COVID disease is output in step 3310.

FIG. 34 is a diagram illustrating an example of the computer system for implementing the present invention. The computer system includes a general purpose computing device in the form of a host computer or a server, on which the steps of the algorithm of FIGS. 31-33 of the present invention are performed. For example, the steps of the algorithm of FIGS. 31-33 of the present invention may be deployed in part or in whole through a device (e.g., smartphone) that executes computer software, program codes, and/or instructions on a processor.

To execute this algorithm, a host computer or a server 3401 includes a central processing unit (CPU) 3402, a system memory 3403, and a system bus 3404 that couples various system components including the system memory to the central processing unit 3402. The system bus 3404 may be any of several types of bus structures including a memory bus or memory controller, a peripheral bus, and a local bus using any of a variety of bus architectures. The system memory includes a read-only memory (ROM) 3405 and random access memory (RAM) 3406. A basic input/output system 3407 (BIOS), containing the basic routines that help to transfer information between the elements within the computer 3401, such as during start-up, is stored in ROM 3405.

The computer or server 3401 may further include a hard disk drive 3408 for reading from and writing to a hard disk, not shown herein, a magnetic disk drive 3409 for reading from or writing to a removable magnetic disk 3410, and an optical disk drive 3411 for reading from or writing to a removable optical disk 3412 such as a CD-ROM, DVD-ROM or other optical media. The hard disk drive 3408, magnetic disk drive 3409, and optical disk drive 3411 are connected to the system bus 3404 by a hard disk drive interface 3413, a magnetic disk drive interface 3414, and an optical drive interface 3415, respectively.

The drives and their associated computer-readable media provide non-volatile storage of computer readable instructions, data structures, program modules, and other data for the server 3401. Although the exemplary environment described herein employs a hard disk (storage device 3416), a removable magnetic disk 3410, and a removable optical disk 3412, it should be appreciated by those skilled in the art that other types of computer readable media that can store data that is accessible by a computer, such as magnetic cassettes, flash memory cards, digital video disks, Bernoulli cartridges, random access memories (RAMs), read-only memories (ROMs) and the like may also be used in the exemplary operating environment.

A number of program modules may be stored on the hard disk (storage device 3416), magnetic disk 3410, optical disk 3412, ROM 3405, or RAM 3406, including an operating system 3417 (e.g., MICROSOFT WINDOWS, LINUX, APPLE OS X or similar). The server/computer 3401 includes a file system 3418 associated with or included within the operating system 3417, such as the Windows NT™ File System (NTFS) or similar, one or more application programs 3419, other program modules 3420, and program data 3421. A user may enter commands and information into the server 3401 through input devices such as a keyboard 3422, a webcam 3423, and pointing device (e.g., a mouse) 3424. Other input devices (not shown) may include a microphone, joystick, game pad, satellite dish, scanner or the like.

These and other input devices are often connected to the central processing unit 3402 through a serial port interface 3425 that is coupled to the system bus, and they may also be connected by other interfaces, such as a parallel port, game port, or universal serial bus (USB). A monitor 3426 or other type of display device is also connected to the system bus 3404 via an interface, such as a video adapter 3427. In addition to the monitor 3426, computers typically include other peripheral output devices (not shown), such as speakers and printers. A host adapter 3428 is used to connect to the storage device 3416.

The server/computer 3401 may operate in a networked environment using logical connections to one or more remote computers 3429. The remote computer (or computers) 3429 may be another personal computer, a server, a router, a network PC, a peer device, or other common network node, and it typically includes some or all of the elements described above relative to the server 3401, although here only a memory storage device 3430 with application software 3419 is illustrated. The logical connections include a local area network (LAN) 3431 and a wide area network (WAN) 3432. Such networking environments are common in offices, enterprise-wide computer networks, Intranets, and the Internet.

In a LAN environment, the server/computer 3401 is connected to the local area network 3431 through a network interface or adapter 3433. When used in a WAN networking environment, the server 3401 typically includes a modem 3434 or other means for establishing communications over the wide area network 3432, such as the Internet. The modem 3434, which may be internal or external, is connected to the system bus 3404 via the serial port interface 3425. In a networked environment, the program modules depicted relative to the computer or server 3401, or portions thereof, may be stored in the remote memory storage device. It will be appreciated that the network connections shown are merely exemplary and other means of establishing a communications link between the computers may be used.

FIG. 35 is a diagram illustrating another example of the computer system for implementing the present invention. For example, an article of manufacture, such as a computer 3501, a memory system, a magnetic or optical disk, some other storage device, or any type of electronic device or system can include one or more processors 3502 coupled to a non-transitory computer-readable medium 3512 such as a memory (e.g., removable storage media, as well as any memory including an electrical, optical, or electromagnetic conductor) having instructions 3513 stored thereon (e.g., computer program instructions), which when executed by the one or more processors 3502 result in performing the steps of the algorithm of FIGS. 31-33 of the present invention.

The computer 3501 can take the form of a computer system having a processor 3502 coupled to a number of components directly, and/or using a bus 3505. Such components can include main memory 3503, static or non-volatile memory 3504, and mass storage 3509. Other components coupled to the processor 3502 can include an output device 3506, such as a video display, an input device 3507, such as a keyboard, a cursor control device 3508, such as a mouse, and a signal generation device 3510 (e.g., a speaker or a light emitting diode (LED)). A network interface device 3511 to couple the processor 3502 and other components to a network 3514 can also be coupled to the bus 3505.

The instructions 3513 can further be transmitted or received over the network 3514 via the network interface device utilizing any one of a number of well-known transfer protocols (e.g., HTTP). While the non-transitory computer-readable medium 3512 is shown as a single medium, the term “computer-readable medium” should be taken to include a single medium or multiple media (e.g., a centralized or distributed database, or associated caches and servers, and or a variety of storage media, such as the processor 3502 registers, memories 3503 and 3504, and the storage device 3509) that store the one or more sets of instructions 3513.

Any of these elements coupled to the bus 3505 can be absent, present singly, or present in plural numbers, depending on the specific embodiment to be realized. In an example, one or more of the processor 3502, the memories 3503 and 3504 storage device 3509 can each include instructions 3513 that, when executed, can cause the computer 3501 to the steps of the algorithm of FIGS. 31-33 of the present invention. In alternative embodiments, the computer 3501 operates as a standalone device or can be connected (e.g., networked) to other machines. In a networked environment, the computer 3501 can operate in the capacity of a server or a client machine in server-client network environment, or as a peer machine in a peer-to-peer (or distributed) network environment. The computer 3501 can include a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine 3501 of the machines that individually or jointly execute a set (or multiple sets) of instructions to perform the steps of the algorithm of FIGS. 31-33 of the present invention.

FIG. 36 is a diagram illustrating yet another example of the computer system for implementing the present invention. The steps of the algorithm of FIGS. 31-33 of the present invention can be implemented as software, in hardware, or as a combination of software and hardware. The computer system for implementing the present invention includes one or more processors, such as a processor 3601. The processor 3601 can be a special purpose or a general purpose digital signal processor. The processor 3601 is connected to a communication infrastructure 3602 (for example, a bus or network). Various software implementations are described in terms of this exemplary computer system. After reading this description, it will become apparent to a person skilled in the relevant art how to implement the disclosed systems and methods using other computer systems and/or computer architectures.

The computer system also includes a main memory 3603, preferably random access memory (RAM), and may also include a secondary memory 3604. The secondary memory 3604 may include, for example, a hard disk drive 3605 and/or a removable storage drive 3606, representing a floppy disk drive, a magnetic tape drive, an optical disk drive, etc. The removable storage drive 3606 reads from and/or writes to a removable storage unit 3607 in a well-known manner. The removable storage unit 3607, represents a floppy disk, magnetic tape, optical disk, etc., which is read by and written to by the removable storage drive 3606. As will be appreciated, the removable storage unit 3607 includes a computer usable storage medium having stored therein computer software and/or data.

In alternative implementations, the secondary memory 3604 may include other similar means for allowing computer programs or other instructions to be loaded into the computer system. Such means may include, for example, a removable storage unit 3608 and an interface 3609. Examples of such means may include a program cartridge and cartridge interface (such as that found in video game devices), a removable memory chip (such as an EPROM, or PROM) and associated socket, and other removable storage units 3608 and interfaces 3609 which allow software and data to be transferred from the removable storage unit 3608 to the computer system.

Computer system may also include a communications interface 3610. Communications interface 3610 allows software and data to be transferred between the computer system and external devices. Examples of communications interface 3610 may include a modem, a network interface (such as an Ethernet card), a communications port, a PCMCIA slot and card, or other communications path interface devices. Software and data transferred via the communications interface 3610 are in the form of signals 3611 which may be electronic, electromagnetic, optical or other signals capable of being received by communications interface 3610. These signals 3611 are provided to communications interface 3610 via a communications path 3612. Communications path 3612 carries signals 3611 and may be implemented using wire or cable, fiber optics, a phone line, a cellular phone link, an RF link and other communications channels.

Computer programs (also called computer control logic) are stored in the main memory 3603 and/or the secondary memory 3604. Computer programs may also be received via the communications interface 3610. Such computer programs, when executed, enable the computer system to implement the steps of the algorithm of FIGS. 31-33 of the present invention. In particular, the computer programs, when executed, enable the processor 3601 to implement the processes disclosed herein. Accordingly, such computer programs operate to control computer system. By way of example, in various exemplary embodiments, the processes/methods performed by signal processing blocks of encoders and/or decoders can be performed by computer control logic. Where the disclosed systems and methods are implemented using software, the software may be stored in a computer program product and loaded into the computer system using the removable storage drive 3606, the hard drive 3605 communications interface 3610, or any other known method of transferring digital information into a computer system.

In this document, the terms computer program medium and computer readable medium are used to generally refer to media such as the removable storage drive 3606, a hard disk installed in hard disk drive 3605, and the signals 3611. These computer program products are means for providing software to the computer system. In another embodiment, disclosed features are implemented primarily in hardware using, for example, hardware components such as Application Specific Integrated Circuits (ASICs) and gate arrays. Implementation of a hardware state machine so as to perform the functions described herein will also be apparent to persons skilled in the relevant art.

FIG. 37 is a diagram illustrating the system for detecting COVID variants according to a first embodiment of the invention. Sensors (including biosensors utilizing two different biological components) 3701 gather biochemical and biophysical data from a person 3702. Also, the biochemical and biophysical data is obtained through laboratory medical tests 3703 and laboratory medical examinations 3704. Laboratory medical tests include tests for COVID disease that can be antigen tests, molecular tests, or antibody tests. All obtained biochemical and biophysical data is combined into a consolidated database 3705 on the server 3706.

Sensors 3701 collect biochemical and biophysical data from a person 3702 representing their symptoms and are either connected to the person 3702 or perform data collection remotely. Sensors 3701 collect the symptom data values from the person for detecting respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

Also, the symptom data values may be obtained at medical institutions that run laboratory medical tests 3703 and laboratory medical examinations 3704. Laboratory medical tests 3703 include the reverse transcription polymerase chain reaction (RT-PCR) test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay. Laboratory medical examinations 3704 include chest CT scans, checking for elevated body temperature, checking for low blood oxygen levels, etc.

The sensors 3701 include a smartphone, pulse oximeter, body temperature thermometer, heart pulse sensor, heart monitor, electrodermal activity (EDA) sensor, respiratory sensor, etc. The sensors 3701 include biosensors of the present invention utilizing two different biological components. The biosensors utilizing two different biological components have two different biological components (e.g., proteins (or protein structures), aptamers, lipids (e.g., sphingoglycolipids, sphingolipids), sugar chains, nucleic acids, DNA, RNA, genes, chromosomes, cell membranes, viruses, antigens, antibodies (or antibody fragments), blood, plasma, blood substitutes, lectins, haptens, hormones, receptors, enzymes, peptides, reagents, polymers, microbial cells, biomolecule dye) that are separated by an internal membrane and coupled to a physicochemical detector or amplifier within the biosensor. Types of biosensors of present invention include those that have proteins or aptamers as the first biological component and use whole cell metabolism, ligand debinding and antibody-antigen reaction. The types of person biological information that can be used for collection of the symptom data values include a whole blood, blood plasma, serum samples, isolated antibodies, blood compositions, blood substitutes, nasal swab, nasopharyngeal swab, oropharyngeal swab, throat swab, deep airway material, saliva.

The server 3706 is connected to sensors and biosensors 3701 via a data exchange system, collecting symptom data values, which includes antibody level, heart rate, blood pressure, pulse oxygen level, respiratory rhythm/rate, etc., and has a network connection to a person's user device. Sensors and biosensors 3701 may be in communication with a smartphone which, in turn, is in communication with at least one computing device via an Internet connection. The computing devices can be of different types, such as a PC, laptop, tablet, smartphone, smartwatch, etc. The process may be performed by the server 3706 or processor in conjunction with the user device (e.g., running a software program provided by the server or processor). Also the symptom data values 3705 can be collected through, manual input into the system, wireless computer protocols, LIS servers, HL7 diagnostic protocols, HIPAA compliant database queries, batch processing from medical records, any other means of digital entry, etc.

All symptom data values collected from person 3702 using sensors and biosensors 3701, medical tests 3703, examinations 3704 are combined into a consolidated database 3705 on the server 3706. The obtained data values within the consolidated database 3705 may provide direct evidence the person 3702 is experiencing one of the symptoms of COVID disease. The server 3706 may operate in a networked environment using logical connections to one or more remote computers. The remote computer (or computers) may be another personal computer, a server, a router, a network PC, a peer device, or other common network node.

The server 3706 is connected with a central site central processing unit and comprises a database 3705 with the received symptom data values and a database 3707 with the predetermined symptom threshold values for SARS-CoV-2 virus strains: original SARS-CoV-2 virus strain, Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc.

Instructions for detecting COVID variants have been programmed according to the computer implemented algorithm that performed by a central processing unit on the server 3706. The algorithm may be implemented on the computer (or another smart device, such as a smartphone, tablet, or laptop) or other software (Cloud server). When implemented on the smartphone, the algorithm may be a component of the application. When implemented on a computer, the algorithm may be a component of a non-transitory computer-readable medium (removable storage drive, a hard disk installed in a hard disk drive, flash memories, removable discs, non-removable discs, etc.) storing a program of instruction.

The algorithm comprises the steps of: receiving a plurality of symptom data values 3705 (the values of the person's biochemical and biophysical data) from a person 3702, calculating the first differentials 3708 by comparing the received values 3705 to predetermined symptom threshold values 3707 for the first original SARS-CoV-2 virus strain (wherein the first differentials 3708 are negative when the values 3705 do not exceed the first predetermined symptom threshold values 3707, and positive when the values 3705 exceed the first predetermined symptom threshold values 3707), using the first differentials to detect the second mutated SARS-CoV-2 virus strain based on a correspondence of its symptoms to the first differentials, calculating the second differentials 3708 by comparing the received values 3705 to predetermined symptom threshold values 3707 for the second SARS-CoV-2 virus strain (wherein the second differentials 3708 are negative when the values 3705 do not exceed the second predetermined symptom threshold values 3707, and positive when the values 3705 exceed the second predetermined symptom threshold values 3707), creating a metric of differentials 3709 in which the first and second differentials 3708 are ordered in their values relative to symptoms of the first SARS-CoV-2 virus strain and symptoms of the second SARS-CoV-2 virus strain, comparing the metric 3709 to the predetermined know metric 3710 that contains known values of differentials indicating that the person has contracted the first or second strain, determining a presence or absence of SARS-CoV-2 in a person 3702.

In an aspect, the determination that the person 3702 has contracted the SARS-CoV-2 virus strain occurs when at least one value within the metric 3709 exceeds the values within the predetermined known metric 3710. In another aspect, the determination that the person 3702 has contracted the SARS-CoV-2 virus strain occurs when the majority of the values within the metric 3709 exceed the values within the predetermined known metric 3710. In an aspect, the second mutated SARS-CoV-2 virus strain is detected such that its symptoms correspond to a majority of the differentials 3708 detected by comparing the received values 3705 to predetermined symptom threshold values 3707 for the first original SARS-CoV-2 virus strain. In another aspect, the second mutated SARS-CoV-2 virus strain is detected such that its symptoms correspond to a minority of the differentials 3708 detected by comparing the received values 3705 to predetermined symptom threshold values 3707 for the first original SARS-CoV-2 virus strain.

A person skilled in the relevant art will recognize other steps may be applied for implementing the algorithm of the present invention. Thus, an algorithm further comprises the step of detecting the third SARS-CoV-2 virus strain based on a correspondence of its symptoms to the second differentials 3708 calculated by comparing the received values 3705 to predetermined symptom threshold values 3707 for the second SARS-CoV-2 virus strain. Then third differentials 3708 (positive or negative) are calculated between the received values 3705 and the predetermined symptom threshold values 3707 for the third SARS-CoV-2 virus strain for further creating the metric of differentials 3709 in which the first, second and third differentials 3708 are ordered in their values relative to symptoms of the first, second and third strain. In an aspect, the third SARS-CoV-2 virus strain is detected such that its symptoms correspond to a majority of the second differentials 3708 detected by comparing the received values 3705 to predetermined symptom threshold values 3707 for the second SARS-CoV-2 virus strain. In another aspect, the third SARS-CoV-2 virus strain is detected such that its symptoms correspond to a minority of the second differentials 3708 detected by comparing the received values 3705 to predetermined symptom threshold values 3707 for the second SARS-CoV-2 virus strain.

In other aspects, an algorithm further comprises the step of identifying for each of a plurality of the first and second differentials 3708 an accurate value indicative of the likelihood that the person 3702 is experiencing the symptom of the first SARS-CoV-2 virus strain or the symptom of the second SARS-CoV-2 virus strain. Then the differentials 3708 within the metric of differentials 3709 are ordered in their accurate values relative to symptoms of the SARS-CoV-2 virus strains. In other aspects, an algorithm further comprises the step of combining the differentials 3708 into multiple groups based on the differences in the differentials 3708. Then the groups of the differentials within the metric of differentials 3709 are ordered relative to symptoms of the SARS-CoV-2 virus strains.

In other aspects, an algorithm further comprises the step of combining the similar symptoms of the SARS-CoV-2 virus strains into a group. Then the differentials 3708 within the metric of differentials 3709 are ordered relative to multiple groups of symptoms. In other aspects, an algorithm further comprises the step of determining the weighting coefficient for each symptom of the SARS-CoV-2 virus strain that characterizes the severity level of symptom. Then the differentials 3708 within the metric of differentials 3709 are ordered relative to the weighting coefficients. In other aspects, an algorithm further comprises the step of determining the complex symptom that shows the correlation of the several symptoms of the SARS-CoV-2 virus strains. Then the differentials 3708 within the metric of differentials 3709 are ordered relative to complex symptoms.

Databases that contain a set 3705 with the symptom data values (the values of the person's biochemical and biophysical data) obtained from sensors and through medical tests that include tests for COVID disease, a set 3707 with the predetermined symptom threshold values for major SARS-CoV-2 virus strains, a set 3708 with the calculated differentials, a set 3709 with the metric of differentials, a set 3710 with the predetermined known metric of differentials are generated and stored to the server 3706. Then, these databases are analyzed using machine learning techniques 3711 that are applied on the data saved on the databases. In another embodiment of the present invention, these databases are uploaded to the Cloud server that is shared by multiple computers.

In response to a determination that the person 3702 has or has not contracted the SARS-CoV-2 virus strain, a diagnosis indicating the presence or absence of a disease is generated. The diagnosis can be a test result indicating the presence or absence of COVID disease. The person's health certificate 3712 includes a QR code capable of being scanned on a user interface and can be tied to the person ID 3713 and used for various digital identifications of the person 3702. Or the number of the person ID 3713 for each respective person 3702 can be electronically tied to their corresponding person's health certificate 3718. In an aspect, the person's health certificate 3712 is storied to a database on a computer for further outputting or displaying. In another aspect, the person's health certificate 3712 is transmitted to a mobile electronic device using end-to-end encryption. In yet another aspect, the person's health certificate 3712 is loaded to a Cloud server that is shared by multiple computers.

FIG. 38 is a diagram illustrating the system for detecting COVID variants according to a second embodiment of the invention. Sensors (including biosensors utilizing two different biological components) 3801 collect biochemical and biophysical data from patients 3802 for detecting symptoms that indicate the presence of a COVID disease. Also, the biochemical and biophysical data is obtained through laboratory medical tests 3803 and laboratory medical examinations 3804. Laboratory medical tests include tests for COVID disease that can be antigen tests, molecular tests, or antibody tests. All obtained biochemical and biophysical data is combined into a consolidated database 3805. The patient's individual data is inputted into the system by the patient 3802 or by a doctor. The patient's individual data includes patient's individual physiological parameters 3806, patient's diseases that accompany COVID-19 3807, additional patient data 3808. All these sets are stored in dataset 3809.

Instructions for calculating differentials have been programmed according to the computer implemented algorithm: comparing the values of the patient's biochemical and biophysical data 3805 to predetermined symptom threshold values for the original COVID-19 virus strain (the first SARS-CoV-2 virus strain) to calculate differentials (positive or negative), using the differentials to detect major COVID variant (the second SARS-CoV-2 virus strain) that is closely related to the patient's disease based on a correspondence of its symptoms to the differentials, comparing the values of the patient's biochemical and biophysical data 3805 to predetermined symptom threshold values for the closely related major COVID variant (the second SARS-CoV-2 virus strain) to calculate differentials (positive or negative). The differentials are negative when the values of the patient's biochemical and biophysical data 3805 do not exceed the predetermined symptom threshold values, and positive when the values of the patient's biochemical and biophysical data 3805 exceed the predetermined symptom threshold values.

In an aspect, the major COVID variant (the second SARS-CoV-2 virus strain) is detected such that its symptoms correspond to a majority of the differential detected by comparing the values of the patient's biochemical and biophysical data 3805 to predetermined symptom threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected by comparing the values of the patient's biochemical and biophysical data 3805 to predetermined symptom threshold values for the original COVID-19 virus strain (the first SARS-CoV-2 virus strain).

In another embodiment of the present invention, instructions further comprises the step of detecting yet another major COVID variant (the third SARS-CoV-2 virus strain) based on a correspondence of its symptoms to the differentials detected by comparing the values of the patient's biochemical and biophysical data 3805 to predetermined symptom threshold values for the closely related major COVID variant (the second SARS-CoV-2 virus strain). Then differentials (positive or negative) are calculated between the values of the patient's biochemical and biophysical data 3805 and the predetermined symptom threshold values for yet another major COVID variant (the third SARS-CoV-2 virus strain).

A set of all differentials 3810 is generated. The set of differentials 3810 is analyzed to detect correlations (tendencies) 3815. To do this, the set of differentials 3810 is complemented by the plurality of the patient's individual data stored in dataset 3809 or by the plurality of the patient's biochemical and biophysical data stored in database 3805. Then the method of combinatorial statistical analysis 3811, the mathematical method of a dense network of curves (method of regression analysis) 3812, the methods of cluster analysis 3813, the machine learning techniques 3814 are used to detect correlations (tendencies). Correlations and tendencies are the mathematical or logical relationships between the values within the set 3810. The resulting plurality of correlations is stored in a database 3815. The algorithm may be implemented on the computer (or another smart device, such as a smartphone, tablet, or laptop) or other software (cloud server). When implemented on the smartphone, the algorithm may be a component of the application. When implemented on a computer, the algorithm may be a component of a non-transitory computer-readable medium (removable storage drive, a hard disk installed in a hard disk drive, flash memories, removable discs, non-removable discs, etc.) storing a program of instruction.

Databases that contain a set of patient's biochemical and biophysical data obtained from sensors and through medical tests that include tests for COVID disease, a set of patient's individual data 3809 (patient's individual physiological parameters 3806, patient's diseases that accompany COVID-19 3807, additional patient data 3808), a set of predetermined symptom threshold values for all major COVID variants, a set of differentials, a set of data about possible post-COVID syndromes are generated. The databases are uploaded and stored to the server 3816. Then, these databases are analyzed using machine learning techniques 3814 that are applied on the data saved on the databases. In another embodiment of the present invention, the databases are uploaded to the cloud server that is shared by multiple computers.

Based on the detected correlations (tendencies) 3815 due to the analysis, a patient's viral disease is diagnosed in the event that at least one correlation (tendency) detected indicates that the patient 3802 is likely to have contracted the COVID disease. In another embodiment of the present invention, the determining that the person has the COVID disease is also based on a confirmation of its symptoms with the values of the patient's biochemical and biophysical data 3805 collected from patients 3802. In another embodiment of the present invention, the detected correlations and tendencies stored in a database 3815 are further analyzed using cluster analysis 3813 to define the same or similar correlations (tendencies) and combining these correlations (tendencies) into a group 3817. The cluster analysis uses the differences in the correlations (tendencies) detected to define multiple groups of correlations (tendencies) 3817 that are same or similar. Based on the multiple groups of correlations (tendencies) 3817 detected, a patient's viral disease is diagnosed in the event that at least one detected group of correlations (tendencies) indicates that the patient 3802 is likely to have contracted the COVID disease.

In response to a determination that the patient 3802 has or has not contracted the disease, a diagnosis indicating the presence or absence of a disease is generated. The diagnosis can be a test result indicating the presence or absence of COVID disease. The system generates diagnosis in the form of a patient's health certificate 3818, which comprises the patient's viral disease as well as all information on the diagnosis, including the test result for COVID disease. The patient's health certificate 3818 includes a QR code capable of being scanned on a user interface and can be tied to the person's ID 3819 and used for various digital identifications of the patient 3802. Or the number of the person's ID 3819 for each respective patient 3802 can be electronically tied to their corresponding patient's health certificate 3818. In an aspect, the patient's health certificate 3818 is storied to a database on a computer for further outputting or displaying. In another aspect, the patient's health certificate 3818 is transmitted to a mobile electronic device using end-to-end encryption. In yet another aspect, the patient's health certificate 3818 is loaded to a Cloud server that is shared by multiple computers.

FIG. 39 is a diagram illustrating the system for detecting COVID variants according to a third embodiment of the invention. Sensors (including biosensors utilizing two different biological components) 3901 collect biochemical and biophysical data from a person 3902. The sensors 3901 are either connected to the person 3902 or perform data collection remotely, and may include a smartphone 3903, a pulse oximeter 3904, a body temperature thermometer 3905, etc., which send the data collected via a secure and encoded channel to a central server 3906. The system includes using a pulse oximeter 3904 to acquire at least the pulse and blood oxygen saturation percentage, which is transmitted wirelessly to a smartphone. The body temperature thermometer 3905 may be any suitable device configured to sense the body temperature and output information indicative of the body temperature. The body temperature thermometer 3905 may output information indicative of the body temperature to the user device 3903 (e.g., smartphone), for example, via direct, short-range, wireless communication signals (e.g., Bluetooth), via the local area network, etc.

The sensors 3901 include biosensors of the present invention utilizing two different biological components. The sensors 3901 may include a electrochemical immunosensor, atomic magnetometer (AM), graphene-based sensor, ion drift sensor, molecular electric transducer (MET), oscillator-based sensor, flame ionization sensor, spectrometer, fluorescence microscope, micro temperature sensor, motion sensor, conductivity sensor, electrical conductivity sensor, electrodermal activity (EDA) sensor, ECG sensor, EMG sensor, a heart pulse sensor, heart monitor, respiratory sensor, etc. For example, data indicative of the heart activity of the person may be received, for example, from the heart pulse sensor. Heart rate variability may be determined, for example, based on data received from the heart monitor. Sensors 3901 may be in communication with a smartphone 3903, which, in turn, is in communication with at least one computing device via a wide area network 3907 (WAN), such as the Internet. The computing devices can be of different types, such as a PC, laptop, tablet, smartphone, smartwatch, etc., using one, or different operating systems or platforms.

The central server 3906 is connected to the sensors 3901 via a data exchange system, collecting biochemical and biophysical data, which includes heart rate, blood pressure, pulse oxygen level, respiratory rhythm/rate, etc. The central server 3906 has a network connection to a user device (e.g., a smartphone 3903) and is connected to the wide area network 3907. The system may also be configured to periodically or continuously monitor the health of the person 3902 (e.g., at least once per day.) It should be appreciated that all data may be acquired manually (e.g., by requiring the person 3902 to enter the information), including respiratory rate (e.g., number of breaths per minute), body temperature, and blood pressure (e.g., systolic pressure, diastolic pressure), and used together with other values, such as Perfusion Index (PI %), Perfusion Index Trend Waveform, age, weight, sex, etc., to determine whether the person 3902 is suffering from a SARS-CoV-2 virus strain. The recording of the data is preferably done through the smartphone 3903, or an application operating thereon, using a simple user interface. Alternatively, the process may be performed by the central server 3906 in conjunction with the user device (smartphone) 3903 (e.g., running a software program provided by the central server 3906.)

The biochemical and biophysical data 3908 may be obtained at a medical institution that runs laboratory medical tests for COVID disease (e.g., antigen test, molecular test, antibody test) and laboratory medical examinations for COVID disease. Laboratory medical tests include the reverse transcription polymerase chain reaction (RT-PCR) test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay, etc. Laboratory medical examinations include chest CT scans, checking for elevated body temperature, checking for low blood oxygen levels, etc.

A set 3909 comprises decrypted all person's biochemical and biophysical data that has been obtained from sensors 3901 and through laboratory medical tests, laboratory medical examinations 3908. Biochemical and biophysical data stored in set 3909 may be obtained by the person 3902 themselves at home, either manually or automatically. This data may be inputted into the system by the person 3902 themselves, or by a doctor using a smartphone 3903 interface. The person's biochemical and biophysical data 3909 are stored on the central server 3906, which is connected to a medical server 3910 and an analytical server 3911, in datasets that are sent to the medical server 3910 and the analytical server 3911.

Separately, a database with predetermined symptom threshold values 3912 for major SARS-CoV-2 virus strains is generated on the medical server 3910. For this, medical guidelines containing up-to-date medical information for definition of predetermined symptom threshold values 3912 for major SARS-CoV-2 virus strains, or the listing of the predetermined symptom threshold values 3912 for major SARS-CoV-2 virus strains are uploaded to the medical server 3910. By relying on well-established, medically documented, famous scientific facts, predetermined symptom threshold values 3912 that indicate a COVID disease can be established.

The threshold value may be determined, based on the latest medical documentation, such that a value of obtained data below the lowest threshold value is indicative of a low likelihood the person 3902 has contracted a COVID disease. The medical guidelines used to determine predetermined symptom threshold values 3912 for major SARS-CoV-2 virus strains may be updated over time. Therefore, the system for detecting new SARS-CoV-2 virus strains provides a platform that can be updated so the predetermined symptom threshold values 3912 reflect the latest understanding of symptoms for major SARS-CoV-2 virus strains. The database comprising predetermined symptom threshold values 3912 for major SARS-CoV-2 virus strains is sent to a machine learning server 3913 for further action.

The medical server 3910 stores primary instructions for processing data in the database with the person's biochemical and biophysical data 3909 and in the database with predetermined symptom threshold values 3912 for major SARS-CoV-2 virus strains. The primary instructions are executed by the medical server 3910 to induce the system for detecting new SARS-CoV-2 virus strains to perform the following steps in accordance with the algorithm: comparing the values of the person's biochemical and biophysical data 3909 to predetermined symptom threshold values 3912 for the first original SARS-CoV-2 virus strain and finding the first differentials (positive or negative), using the first differentials to detect the second mutated SARS-CoV-2 virus strain based on a correspondence of its symptoms to the first differentials, comparing the values of the person's biochemical and biophysical data 3909 to predetermined symptom threshold values 3912 for the second SARS-CoV-2 virus strain and finding the second differentials (positive or negative).

In an embodiment of the present invention, the second mutated SARS-CoV-2 virus strain is defined such that its symptoms correspond to a majority of differentials detected by comparing the values of the person's biochemical and biophysical data 3909 to predetermined symptom threshold values 3912 for the first original SARS-CoV-2 virus strain. In another embodiment of the present invention, the second mutated SARS-CoV-2 virus strain is defined such that its symptoms correspond to a minority of differentials detected by comparing the values of the person's biochemical and biophysical data 3909 to predetermined symptom threshold values 3912 for the first original SARS-CoV-2 virus strain.

Set 3914 is a set of differentials detected based on the predetermined symptom threshold values 3912 for major SARS-CoV-2 virus strains conforming to medical guidelines that have been obtained by executing the primary instructions. Differentials can be both positive and negative. The differentials are negative when the values of the data 3909 received do not exceed the predetermined symptom threshold values 3912, and positive when the values of the data 3909 received exceed the predetermined symptom threshold values 3912. The set of differentials 3914 is stored in a database and sent to the analytical server 3912 for further analysis, as well as to the machine learning server 3913.

The analytical server 3911 stores the following databases: a database with person's biochemical and biophysical data 3909, a database with predetermined symptom threshold values 3912 for major SARS-CoV-2 virus strains, a database with differentials 3914 that have been determined using the primary instructions. The analytical server 3911 executes secondary instructions stored on the server, applying them to all data in the databases listed above. The secondary instructions induce the system to perform the following operations in accordance with the algorithm: creating a metric of differentials 3915 in which the differentials from the set of differentials 3914 are ordered in their values relative to symptoms of the SARS-CoV-2 virus strains, comparing the metric 3915 to the predetermined known metric that contains known values of differentials indicating that the person has contracted the SARS-CoV-2 virus strain, determining a presence or absence of SARS-CoV-2 in a person.

The created metric of differentials 3915 is stored in a database that is sent to a certification server 3916 for further actions and to the machine learning server 3913 to create machine learning techniques 3917. Also, the metric of differentials 3915 is stored on a non-transitory computer-readable medium (removable storage drive, a hard disk installed in hard disk drive, flash memories, removable discs, non-removable discs, etc.), a Cloud server, a computer, or any other equivalent device. In another embodiment of the present invention, the algorithm further comprises the steps of: creating a metric of differentials 3915, analyzing the metric of differentials 3915 to detect tendencies (correlations) within the metric indicative of relationships between the differentials, determining if the person 3902 has the first SARS-CoV-2 virus strain or the second SARS-CoV-2 virus strain when at least one detected tendency indicates the person 3902 has contracted the first or second virus strain, outputting a result indicating a presence or absence of SARS-CoV-2 in a person.

The machine learning server 3913 stores the following databases: a database with the person's biochemical and biophysical data 3909, a database with predetermined symptom threshold values 3912 for major SARS-CoV-2 virus strains, a database with differentials 3914 that have been calculated using the primary instructions, a database with metric of differentials 3915 that have been calculated using the secondary instructions, a database with predetermined known metric. The machine learning server 3913 applies machine learning techniques 3917 to all data stored in the databases listed above for detecting correlations between the differentials.

Also, machine learning techniques 3917 allow us to set predetermined symptom threshold values 3912 for major SARS-CoV-2 virus strains, and to update and/or adjust predetermined symptom threshold values 3912 for major SARS-CoV-2 virus strains. New predetermined symptom threshold values 3912 are sent to the medical server 3910 to update medical guidelines for major SARS-CoV-2 virus strains. The reports and graphs from machine learning server 3913 are stored on the cloud server and certification server 3916 for conclusions and suggestions, as well as on a non-transitory computer-readable medium.

Based on the comparing the metric 3915 to the predetermined known metric that contains known values of differentials indicating that the person has contracted the SARS-CoV-2 virus strain that have been calculated using the secondary instructions, diagnosis is performed on the certification server 3916 having a network connection with the user device 3903 (e.g., the smartphone), in which a software application is run. Software can be used to make a medical diagnosis based on the received information to determine the likelihood that the person 3902 has contracted the SARS-CoV-2 virus strain. The certification server 3916 analyses data entries, electronically, to find information to determine if the person 3902 has actually been infected by a SARS-CoV-2 virus strain or not and to determine whether there is information confirming infection by a SARS-CoV-2 virus strain. If yes, the certification server 3916 generates a diagnosis providing the projected patient's health condition in real-time.

The diagnosis is displayed on the smartphone 3903 screen, and the system may determine the likelihood that the person 3902 has contracted the SARS-CoV-2 virus strain in response to a request by the person 3902 (e.g., via the user device graphical interface.) The results provided to the person 3902 could be an indication (positive, negative), the likelihood (1-10, low, medium, high), the disease severity (uninfected, mild, moderate, and severe), etc. Also, the diagnosis can be provided as a person's health certificate 3918. The person's health certificate 3918 includes a QR code capable of being scanned to display the person's health certificate 3918 on a graphical interface on the user's electronic device 3903 (e.g., the smartphone). The person's health certificate 3918 comprises the representation of the person's biometric sample, which is one or more thumbprint sets, a retina scan, a DNA sample, etc.

The certification server 3916 can be communicatively coupled to an internal API for transmission of person's health certificates 3918 to electronic medical records and human resources records in medical institutions. External APIs can be communicatively coupled to the certification server 3916 to query the person's health certificates 3918 associated with the person 3902. For external APIs, the system can output the necessary information based on the type of entity requesting the information. For example, the system can output to a requestor via a graphical representation or report on a smartphone 3903 the person's health certificate 3918. The number of the person ID 3919 for each respective person 3902 can be electronically tied to their corresponding person's health certificate 3918. The person ID 3919 can be used as a unique electronic element or identifier to access subsequent queries for the person's health certificate 3918 of the person 3902. While a preferred embodiment has been set forth above, those skilled in the art will readily appreciate that other embodiments can be realized within the represented diagram of the system.

FIG. 40 is a diagram illustrating the system for detecting COVID variants according to a fourth embodiment of the invention. Multiple collectors 4001, 4002, 4003 and 4004 would be located and mounted on a known walk-through metal detector 4005, such as shown in FIG. 40. Each collector would include an air vacuum or be attached to a central air vacuum box 4006 which would gather and pull in air surrounding the collector. Accordingly, as a person 4007 passed through the walk-through detector 4005, air would be sampled in the immediate vicinity of the individual passing therethrough. By way of example and not by way of limitation, the collectors might be located approximately 25 to 50 cm away from the individual. The particular location would vary depending on the mounting location and depending on the sensitivity of the collector.

Each collector would be connected by a tube or passageway to a sensor 4008 or sensor located nearby. Accordingly, an airborne specimen is obtained. Once the collectors have gathered an airborne specimen or sample, the particulate matter in the specimen will be analyzed by the sensor or biosensors. As the values of the data are obtained, it will be transmitted via a transmitting system central processing unit 4009 to a server 4008 for analysis. A benefit of the present invention is that it could be employed with existing metal detectors in place which would be in close proximity to those passing into and through airports and government buildings. Accordingly, the structure for deploying such a system is already in place.

Instructions for detecting COVID variants have been programmed according to the computer implemented algorithm that performed by a central processing unit on the server 4008. The algorithm comprises the steps of: calculating the first differentials (positive or negative) by comparing the values received by collectors 4001, 4002, 4003 and 4004 to predetermined symptom threshold values for the first original SARS-CoV-2 virus strain, using the first differentials to detect the second mutated SARS-CoV-2 virus strain based on a correspondence of its symptoms to the first differentials, calculating the second differentials (positive or negative) by comparing the values received by collectors 4001, 4002, 4003 and 4004 to predetermined symptom threshold values for the second SARS-CoV-2 virus strain, creating a metric of differentials in which the first and second differentials are ordered in their values relative to symptoms of the first SARS-CoV-2 virus strain and symptoms of the second SARS-CoV-2 virus strain, comparing the metric to the predetermined metric that contains known values of differentials indicating that the person has contracted the first or second strain, determining a presence or absence of SARS-CoV-2 in a person 4007.

In another embodiment of the present invention, the algorithm further comprises the steps of: creating a metric of differentials, analyzing the metric of differentials to detect tendencies (correlations) within the metric indicative of relationships between the differentials, determining if the person 4007 has the first SARS-CoV-2 virus strain or the second SARS-CoV-2 virus strain when at least one detected tendency indicates the person 4007 has contracted the first or second virus strain, outputting a result indicating a presence or absence of SARS-CoV-2 in a person 4007.

FIG. 41 is a diagram illustrating the system for detecting COVID variants according to a fifth embodiment of the invention. Components 4101-4104 of the system represent a “testing” phase, components 4105-4107 of the system represent an “analysis” phase, and components 4108-4110 represent a “realization” phase of giving the person's certificate. In the “testing” phase, the person's antibody data through medical tests can be collected in real-time. A variety of manual and/or automated medical testing can transmit a person's antibody data to the system. The all person data could include, but is not limited to, manual and automatic laboratory medical test 4101 (e.g., antigen or antibody test) in the medical institutions, private medical test 4102 (e.g., private testing) and person's individual data 4103 (e.g., the person's individual physiological parameters, the person's diseases that accompany COVID disease).

The laboratory medical tests 4101, private medical tests 4102, and the person's individual data 4103 can be used to collect data on a variety of persons. The antibody data can be collected from a medical tests 4101 and 4102 in real-time, and/or from already administered tests. The types of person's biological information that can be used for collection of the antibody data can include a whole blood, blood plasma, serum samples, isolated antibodies, blood compositions, blood substitutes, nasal swab, nasopharyngeal swab, oropharyngeal swab, throat swab, deep airway material, saliva. The person's antibody data can be collected through, e.g., manual input into the system, wireless computer protocols, LIS servers, HL7 diagnostic protocols, HIPAA compliant database queries, batch processing from medical records, any other means of digital entry, etc.

All antibody data obtained through medical tests 4101 and 4102 are combined into a consolidated database 4104. A consolidated database 4104 maintaining person's antibody data is coupled to the analytical server 4105 and is updated in real-time. Configurable instructions for analysis of the data (partially or fully incorporated in the analytical server 4105) having differentials calculations logic programmed therein can receive as input the person's antibody data from consolidated database 4104, the person's individual data 4103, the predetermined symptom threshold values 4106, and detect the values of the differentials 4107.

An analytical server 4105 comprises a set of instructions for: calculating the first differentials 4107 (positive or negative) by comparing the values of the person's antibody data saved in consolidated database 4104 to predetermined IGM and IGG antibody threshold values 4106 for the first original SARS-CoV-2 virus strain, using the first differentials to detect the second mutated SARS-CoV-2 virus strain based on a correspondence of its symptoms to the first differentials, calculating the second differentials 4107 (positive or negative) by comparing the values of the person's antibody data saved in consolidated database 4104 to predetermined IGM and IGG antibody threshold values 4106 for the second SARS-CoV-2 virus strain, creating a metric of differentials in which the first and second differentials are ordered in their values relative to symptoms of the first SARS-CoV-2 virus strain and symptoms of the second SARS-CoV-2 virus strain, comparing the metric to the predetermined metric that contains known values of differentials indicating that the person has contracted the first or second strain, determining a presence or absence of SARS-CoV-2 in a person.

In another embodiment of the present invention, the algorithm further comprises the steps of: creating a metric of differentials, analyzing the metric of differentials to detect tendencies (correlations) within the metric indicative of relationships between the differentials, determining if the person has the first SARS-CoV-2 virus strain or the second SARS-CoV-2 virus strain when at least one detected tendency indicates the person has contracted the first or second virus strain, outputting a result indicating a presence or absence of SARS-CoV-2 in a person.

A certification server 4108 can receive as input the consolidated diagnosis from the analytical server 4105, and generates a person's health certificate 4109 for each patient based on this diagnosis. The person's health certificate 4109 comprises the representation of the biometric sample of the person. The biometric sample is one or more of a thumbprint set recorded from the person, a retina scan recorded from the person, and a DNA sample obtained from the patient and analyzed, etc. The person's health certificate 4109 includes a code (i.e., a QR code) capable of being scanned to display the health certificate on user interface at the electronic device 4110.

The system can receive as input at the certification server 4108 the values of the differentials 4107 from the analytical server 4105 in real-time to assist in generation of the diagnosis for the person. The differentials calculations logic programmed can include, e.g., medical guidelines on predetermined symptom threshold values 4106 for major SARS-CoV-2 virus strains. The differentials are calculated between the values of the person's antibody data at the consolidated database 4104 and predetermined symptom threshold values 4106 for SARS-CoV-2 virus strains to define the person's COVID disease. The values of the differentials 4107 can be updated in real-time as a set of rules for the probability of being infected by a new SARS-CoV-2 virus strain based on new values of person's antibody data in updated database 4104. Operation of the certification server 4108 is thereby dynamic based on ongoing changes in the values of the differentials 4107, as well as updated medical tests 4101 and 4102 for the person received at the analytical server 4105 in real-time.

In addition to using the person's antibody data saved in consolidated database 4104 and the person's individual data 4103 to analyze and determine the diagnosis for the person and the person's health certificate 4109, the system can receive as input an aggregate from other systems to assist in the analysis of the COVID disease. The diagnosis can be output by the system as safety levels of the probability of being infected by a new SARS-CoV-2 virus strain for the person, e.g., with Level 1 being the safest, and each increasing level representing an additional level of risk of the COVID viral disease that infects the person.

With respect to the major SARS-CoV-2 virus strains, the analytical server 4105 can used antibody test data for the analysis, as well as person's individual data. In one instance, the certification server 4108 analysis can be as follows: COVID disease IGG+ with IGG index>20 and no prior conditions, IGM negative with index<0.5, body temperature<37.8° C., and the certification server 4108 to output a viral disease Level 1 or safest level for the patient. In another instance, the analytical server 4105 analysis can be as follows: COVID disease IGG+ with IGG index>20 and no prior conditions, IGM negative with index<1, body temperature<37.2° C., and certification server 4108 to output a viral disease level of 2 or the next safest level for the person. The person's health certificate 4109 with safety level of the viral disease can be automatically updated in real-time based on additional medical tests 4101 and 4102 received by the system for the person and/or based on updated the values of the differentials 4107, person's individual data 4103.

The system can be initially programmed to a very high level of IGG antibodies and a very low level of IGM antibodies to denote a low COVID disease risk level. Such antibody levels can be used as predetermined symptom threshold values 4106 for analysis by the analytical server 4105. As the person's antibody data changes and as the system learns more about the disease through the updated the values of the differentials 4107, the diagnosis can be adjusted and applied to the existing dataset. The system can thereby continuously or substantially continuously update the risk level of the person's COVID viral disease based on the updated the values of the differentials 4107 and/or based on medical test data previously received by the system, reducing the need to re-test person if the data is available. In some embodiments of the present invention, such updating by the system can be based on the type of medical tests 4101 and 4102 taken previously by the person. For example, a re-test may be necessary to determine the current level of antibodies of the person. The continuously or substantially continuously updating of the medical tests 4101 and 4102 can be similarly used for any person's antibody data being recorded and aggregated into consolidated database 4104 that may inform the viral disease score determination.

The certification server 4108 can be communicatively coupled to an internal API for transmission of a person's health certificate 4109 to electronic medical records and human resources records in the medical institutions. External APIs can be communicatively coupled to the certification server 4108 to query the certification server 4108 for the person's health certificate 4109 associated with person. For external APIs, the system can output the necessary information based on the type of entity requesting the information. For example, the system can output to a requestor an indication that the patient is safe or not safe. As a further example, the system can output to a requestor via a graphical representation on an electronic device 4110 a person's health certificate 4109 with the diagnosis, whether the person is safe or not safe, and additional information regarding person's disease of major SARS-CoV-2 virus strain.

The person's health certificate 4109 generated for each person can be electronically stored in the certification server 4108. The generated person's health certificate 4109 can also be tied to the person ID base used for various digital identifications of the person. The person's ID number for each respective person can be electronically tied to their corresponding person's health certificate 4109. In this case, the person's ID can be used as a unique electronic element or identifier to access with subsequent queries for the person's health certificate 4109 of the person. For example, the person's health certificate 4109 together with the person's ID may be employed to access for a person to a public place with a large number of people, where permission to enter is required and where the spread of a viral infection is of great danger, for example, a stadium or an airplane.

In another embodiment of the present invention, the person associated with the encrypted unique person ID number can be provided with a selection to allow the person to electronically decide who has access to the information at the person's health certificate 4109 and for what purpose. For example, the person can selectively choose who has access to the person's health certificate 4109 and associated information on a case-by-case basis. In another embodiment of the present invention, the person can provide a one-time confirmation to allow all future access to the information at the person's health certificate 4109 until the person decides to opt-out of such access.

FIG. 42 is a diagram illustrating the system for detecting COVID variants implemented in the device known as a “Covidometer.” The “Covidometer” comprises a collector (e.g., a sensor chip or biochip) 4201 that collects a blood or sputum sample 4202 from the person 4203, a plurality of sensors and biosensors 4204 (e.g., a sensors panel) that gather data values from the sample 4202, a transmission system 4205 to transmit data values from the sensors and biosensors 4204, a central processing unit 4206 in communication with the transmission system 4205 to collect data values from the sensors and biosensors 4204, a storage 4207 that receives and stores sample 4202, a server 4208 in communication with the central processing unit 4206 that comprise a database 4209 with gathered data values and a database 4210 with predetermined symptom threshold values for SARS-CoV-2 virus strains, a software application 4211 that is downloadable to and executable by the central processing unit 4206 to detect tendencies, means (e.g., a display or printer) 4212 for outputting a result indicating a presence or absence of COVID disease in a person 4203.

The collector (e.g., the sensor chip or biochip) 4201 has incorporated thereon a number of biosensors 4213-4216 utilizing two different biological components, which are mounted thereon. The biosensors 4213-4216 interact with a sample 4202 (e.g., a whole blood, blood plasma, serum samples, isolated antibodies, blood compositions, blood substitutes, nasal swab, nasopharyngeal swab, oropharyngeal swab, throat swab, deep airway material, saliva) from the person 4203, which will be analyzed by sensors 4217-4220, which are installed on the sensors panel to gather data values from the sample 4202. In some aspects, the biosensors 4213-4216 work based on the use whole cell metabolism, ligand debinding and antibody-antigen reaction. The biosensors utilizing two different biological components produce electrical, magnetic or optical signals detectable by the sensors 4217-4220 that can be integrated into the memory device of FIG. 9 or can be integrated into the internal membrane of FIG. 10.

Once the collector (the sensor chip or biochip) 4201 has gathered a sample 4202 from the person 4203, the sample 4202 will be delivered in the storage 4207 that receives and stores collected the blood or sputum sample from the person. The purpose of the storage 4207 (e.g., the box for biological materials or blood flask storage cabinet) is to provide for preservation of the sample 4202 of the person 4203 to enable it to be reanalyzed by the “Covidometer.” The analysis of the sample 4202 will result in sending data values gathered from the sample 4202 by using the sensors 4217-4220 to a server 4208. The sensors 4217-4220 installed in the sensors panel 4204 of the “Covidometer” are replaceable so that a failure of any sensor could be addressed by a simple replacement of the sensor, allowing simple and robust connection with hardware components of the “Covidometer” by common protocols and procedures following universal standards. In some aspects, the sensors 4217-4220 are embedded in the memory device within the “Covidometer.”

The process to collect data values from the sample 4202 stored in the storage 4207 by using the sensors 4217-4220 may follow a process having a number of steps. At the initial stage data values will be retrieved from the sample 4202 of person 4203. At the next stage the symptom data values representing the COVID symptoms of person 4203 will be determined from the data values retrieved from the ample 4202. The COVID symptoms of person 4203 may be respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

The transmission system 4205 transmit the symptom data values gathered from the sensors 4217-4220, and thereafter the central processing unit 4206 in communication with the transmission system 4205 collect the symptom data values. The central processing unit 4206 is a transmitting or monitored central processing unit which is connected through the transmission system 4205 to biosensors utilizing two different biological components 4213-4216 and sensors 4217-4220. In an aspect, the transmitting or monitored central processing unit will be connected to a transmission system 4205, e.g., standard telecommunication network, and thereafter the symptom data values will be delivered to the central processing unit 4206. The symptom data values are transferred by the sensors 4217-4220 using a secure encoded channel, and levels of encryption are applied to all data transfer. In another aspect, the “Covidometer” may further include the transmitting system central processing unit remote from the central processing unit 4206. For example, the transmitting system central processing unit may be installed on a collector (biochip) 4201.

The central processing unit 4206 is connected to a transmission system 4205, and thereafter all the symptom data values gathered by using the biosensors 4213-4216 and sensors 4217-4220 will be delivered to a server 4208. The server 4208 in communication with the central processing unit 4206 comprises the database 4209 with the gathered symptom data values and the database 4210 with predetermined symptom threshold values for SARS-CoV-2 virus strains: Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc.

A software application 4211 receives information and executing by the central processing unit 4206 within the server 4208 comprised the databases 4209 and 4210, on which steps are performed according to the algorithm to manipulate the databases 4209 and 4210 of medical data for detect tendencies within the symptom data values. One of the ordinary skills in the art will further understand the various programming languages that can be employed to create one or more the software applications 4211 designed to implement and perform the steps of the algorithm. For example, the software applications 4211 can be structured in an object-orientated format using an object-oriented language, such as Java, C++, or one or more other languages. Alternatively, the software applications 4211 can be structured in a procedure-orientated format using a procedural language, such as assembly, C, etc.

The algorithm to detect tendencies comprises the steps of: calculating the first differentials (positive or negative) by comparing the received values stored in the database 4209 to predetermined symptom threshold values for the first original SARS-CoV-2 virus strain stored in the database 4210, using the first differentials to detect the second mutated SARS-CoV-2 virus strain based on a correspondence of its symptoms to the first differentials, calculating the second differentials (positive or negative) by comparing the received values stored in the database 4209 to predetermined symptom threshold values for the second SARS-CoV-2 virus strain stored in the database 4210, creating a metric of differentials in which the first and second differentials are ordered in their values relative to symptoms of the first SARS-CoV-2 virus strain and symptoms of the second SARS-CoV-2 virus strain, comparing the metric to the predetermined metric that contains known values of differentials indicating that the person has contracted the first or second strain, determining a presence or absence of SARS-CoV-2 in a person 4203, outputting a result indicating a presence or absence of COVID disease in a person 4203 by using the means 4212.

In another embodiment of the present invention, the algorithm further comprises the steps of: creating the metric of differentials, analyzing the metric of differentials to detect tendencies indicative of relationships between the differentials within the metric, determining if the person has the first SARS-CoV-2 virus strain or the second SARS-CoV-2 virus strain when at least one detected tendency indicates the person 4203 has contracted the first or second virus strain, outputting a result indicating a presence or absence of COVID disease in a person 4203 by using the means 4212.

Means 4212 outputs a result indicating a presence or absence of COVID disease in a person 4203. Means 4212 for outputting a result are an electronic device (such as a liquid-crystal display (LCD)) or part of a device (such as the screen of a tablet) that presents the result in visual form for patient 4203. Means 4212 can be any piece of computer hardware equipment which converts information into a human-perceptible form, such as text, graphics, audio, or video. Examples of means 4212 include monitors (e.g., a computer monitor or studio monitor), speakers (e.g., computer speakers), headphones, projectors (e.g., a LED projector), printers (e.g., inkjet printers, laser printers, thermal printers, dot matrix printers), tactile displays, braille displays, terminals for outputting information (e.g., a monochromatic terminal), punched cards, etc. It should be understood that other and further modifications of the “Covidometer,” apart from those shown or suggested herein, may be made within the spirit and scope of the present invention.

FIG. 43 is a diagram illustrating hardware components for implementing the device of the “Covidometer.” The system for detecting COVID variants of the present invention can be implemented in the device of the “Covidometer” that determines if a person has a viral disease of a first original COVID-19 virus strain or second mutated COVID variant and requires no lab work. The “Covidometer” of FIG. 43 is the small, portable battery powered device 4301 with computing resources in the form of a storage 4302 for blood and sputum samples (e.g., a box for biological materials), processor 4303, memory device 4304 with the sensors 4305 integrated into the memory device 4304 (in another embodiment, the sensors 4305 can be integrated into the internal membrane separating two biological components within the device 4301), analyzer 4306 to detect tendencies, screen 4307, and it can determine at home whether a person has contracted COVID disease.

FIGS. 44-46 illustrate examples of the operation of the device of the “Covidometer.” As shown in FIG. 44, the “Covidometer” 4401 is configured to cooperate with the blood or sputum sample 4402 by using biosensors 4403 utilizing two different biological components. Types of biosensors 4403 utilizing two different biological components of the “Covidometer” 4401 include those that use whole cell metabolism, ligand debinding and antibody-antigen reaction. The types of blood or sputum samples 4402 of a person that can be used for collection of the symptom data values include a whole blood, blood plasma, serum samples, isolated antibodies, blood compositions, blood substitutes, nasal swab, nasopharyngeal swab, oropharyngeal swab, throat swab, deep airway material, saliva.

The whole sampling process is carried out by the biosensors 4403 utilizing two different biological components. The biosensors 4403 produce an electrical, magnetic or optical signals (e.g., electrochemical change to detect presence of antibodies) detectable by sensors 4404 (e.g., the electrochemical immunosensors capable of distinguishing IgM and IgG antibodies from each other) installed in the device of the “Covidometer” 4401. In an embodiment, the sensors 4404 may be integrated and embedded into a memory device within the “Covidometer” 4401. In another embodiment, the sensors 4404 may be integrated and embedded into an internal membrane separating two biological components within the “Covidometer” 4401.

The biosensors 4403 utilizing two different biological components uses aptamers as the first biological component, which are short, artificially synthesized pieces of DNA or RNA that specifically interact with the blood or sputum sample 4402. In some aspects, the biosensors 4403 utilizing two different biological components are applied to a replaceable biochip coated with a conductive layer of reduced graphene oxide. When aptamers bind to blood or sputum proteins 4402, they gain or lose an extra electron, and this change the resistance of the conductive layer. The current passing through it increases or decreases, which is recorded by the sensors 4404 (e.g., the electrochemical immunosensors) of the portable device 4401.

The principle of operation of the “Covidometer” 4401 is similar to the measurement of blood sugar using a glucometer. To determine at home whether a person has contracted SARS-CoV-2 virus strain, the person needs to drop blood 4402 (or place sputum 4402) on the biosensors 4403. When the first biological component of the biosensors 4403 interacts with a sample 4402 containing excess predetermined protein threshold values, the electrical conductivity of the biosensors 4403 changes, what will be recorded by the sensors 4404. Thereafter the data gathered by using the sensors 4404 will be analyzed in the “Covidometer” to identify that the person has the SARS-CoV-2 virus strain in response to the sensors 4404 detecting a value that is greater than or less than the thresholds for the sensors 4404. After a few minutes, the screen of the “Covidometer” 4401 will display the result indicating a presence or absence of COVID disease in a person.

In another embodiment of the present invention, an aptamer is tagged with a fluorescent tag and mixed with blood or sputum samples 4402, where it finds viral proteins, binds to them and visually shows their presence in the blood or sputum 4402. Aptamers literally “illuminate” them due to fluorescence. To do this, the “Covidometer” 4401 uses a replaceable electrochemical biochip 4403, onto which several types of aptamers with fluorescent tags “attached” to them can be applied at once, and thus conduct a comprehensive examination of a sample 4402 obtained from a person. In another embodiment of the present invention, biosensors 4403 utilizing two different biological components use a dye as the second biological component. This combination of aptamer (DNA) and dye-biomolecule can significantly improve the effectiveness of the “Covidometer” 4401 with blood or sputum sample 4402 in detecting COVID disease in a person.

FIG. 45 is a diagram illustrating another example of the operation of the device of the “Covidometer.” The “Covidometer” for detecting SARS-CoV-2 in sample obtained from a person is based on magnetic particle spectroscopy technology (MPS). The “Covidometer” has a tube 4501 containing tiny magnetic iron oxide nanoparticles 4502 suspended in a liquid. Magnetic nanoparticles 4502 are coated with molecules, such as antibodies 4503, that recognize and attach to pieces of protein unique to the SARS-CoV-2 virus strain. The “Covidometer” testing involves taking blood or sputum sample from a person and mixing them with two biological components of the biosensor 4504 utilizing two different biological components, which destroy any viral particles. This releases viral proteins and genetic material (RNA) and makes these viral targets accessible to antibodies 4503.

The “Covidometer” then applies a magnetic field to the tube 4501. One part of each viral protein will stick to the specific antibody 4503 on the surface of the magnetic nanoparticle 4502, and the other part will hang freely. The dangling part can stick to another antibody 4503 on another magnetic nanoparticle 4502, turning the protein into a bridge holding the two magnetic nanoparticles together. As this process is repeated, clumps of magnetic nanoparticles are formed, causing the magnetic signals 4505 emanating from the tube 4501 to weaken, that is detected by detectors 4506 (by detectors integrated into the memory device of the “Covidometer”, or by detectors integrated into the internal membrane separating two biological components within the “Covidometer”) and gives a positive test result by the “Covidometer.”

In another embodiment of the present invention, the basis of the “Covidometer” is a silicon chip with two electrodes, a nanowire or nanoribbon, onto which antibodies 4503 are applied. The principle of operation of the “Covidometer” is similar to the example noted above, but is based on recording signals of electric current flowing through the nanowire or nanoribbon. When viral protein particles of blood or sputum enter the “Covidometer”, they bind to antibodies applied to the surface, what changes the electrical conductivity of the nanowire or nanoribbon, what is detected by detectors 4506 (by detectors integrated into the memory device of the “Covidometer”, or by detectors integrated into the internal membrane separating two biological components within the “Covidometer”) and gives a positive test result by the “Covidometer.”

FIG. 46 is a diagram illustrating yet another example of the operation of the device of the “Covidometer.” The disposable test strip 4601 is inserted into the “Covidometer” for testing, which analyzes blood or saliva samples obtained from a person. Blood or saliva 4602 is applied to the end of the strip 4601 and the result appears on the screen of the “Covidometer” within seconds. To set up such a system for detecting SARS-CoV-2 in blood or saliva 4602, the “Covidometer” uses a biosensor 4603 utilizing two different biological components, where proteins 4604 and antibodies 4605 are the first and second biological components, respectively, which are separated by an internal membrane 4606 within the biosensor 4603.

The antibodies 4605 bind specifically to the virus in blood or saliva 4602, resulting in a series of chemical reactions to attach the antibodies 4605 to the strip 4601. Once inserted into the “Covidometer”, the strip 4601 with proteins 4604 and antibodies 4605 is exposed to an electrical current 4607 generated by the internal circuit board 4608. If blood or saliva 4602 applied to the strip 4601 contains SARS-CoV-2, then the viral particles bind to the proteins 4604 and antibodies 4605, slightly deforming them in this process. These subtle movements create distortions in the electrical current 4607 (the more virus, the more distortion) what is detected by detectors 4609 within the “Covidometer” (by detectors integrated into the memory device of the “Covidometer”, or by detectors integrated into the internal membrane separating two biological components within the “Covidometer”) into numerical values that appear on the screen of the “Covidometer.”

In another embodiment of the present invention, the “Covidometer” of the present invention can detect not only the basic original SARS-CoV-2 virus strain, but also its derivative strains within blood or saliva 4602, as it analyzes angiotensin, —converting enzyme 2 (ACE2), —a viral receptor that is common to all known major SARS-CoV-2 virus strains. To do this, the biosensor 4603 uses proteins 4604 that glow when mixed with virus components in blood or saliva 4602. The protein constructs 4604 recognize specific molecules on the surface of a virus or antibody, bind to them, and then emit light as a result of a biochemical reaction. This optical signal is detected by detectors 4609 (by detectors integrated into the memory device of the “Covidometer”, or by detectors integrated into the internal membrane separating two biological components within the “Covidometer”) what gives a positive test result by the “Covidometer.”

Having thus described a preferred embodiment, it should be apparent to those skilled in the art that certain advantages of the described method and apparatus have been achieved. While various embodiments of the present invention have been described above, it should be understood that they have been presented by way of example only, and not limitation, and therefore the examples and embodiments described herein are non-limiting examples. A person skilled in the relevant art will recognize that other components and configurations may be used without parting from the spirit and scope of the embodiments. Furthermore, the various features of the embodiments described herein may be extracted and/or combined to form new embodiments, and each specific element includes all technical equivalents that operate in a similar manner to accomplish a similar purpose.

In the drawings and the description of the drawings herein, certain terminology is used for convenience only, and is not to be taken as limiting the embodiments of the present invention. References to “one embodiment,” “an embodiment,” etc., may indicate that the embodiment(s) of the invention so described may include a particular feature, structure, or characteristic, but not every embodiment necessarily includes the particular feature, structure, or characteristic. The terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. Also, in the following claims, the terms “including” and “comprising” are open-ended, that is, a system, device, article, or process that includes elements in addition to those listed after such a term in a claim are still deemed to fall within the scope of that claim.

What is claimed is:

1. A system for determining COVID disease in a person, the system comprising:
(a) a protein biosensor configured to:
interact with a blood sample obtained from a person and change a chemical structure of the blood sample;
contain two compositionally different biological components, wherein:
the first biological component and the second biological component are coupled to the same transducer within the protein biosensor;
the first biological component and the second biological component are not in contact and are separated from each other by an internal membrane within the protein biosensor; and
the first biological component represents proteins and the second biological component is different from the first biological component in chemical composition;
contain the internal membrane, wherein the internal membrane:
is located between the first and second biological components, functioning as a physical barrier between them; and
has a first contact surface with the first biological component and a second contact surface with the second biological component;
output both the first signal and the second signal, respectively, when the first biological component and the second biological component change the chemical structure of the blood sample, wherein:
the first and second signals comprise measurable parameters; and
the second signal is different from the first signal;
(b) a wireless communication device configured to communicate using a wireless peer-to-peer or machine-type-communication protocol;
(c) a memory device having one or more integrated detectors, the memory device being configured to:
receive a first signal and a second signal from the protein biosensor via the one or more integrated detectors;
wherein the one or more integrated detectors are embedded into the memory device and are configured to decrypt the first or the second signal into corresponding values;
include a memory device controller that is coupled to each of the one or more integrated detectors;
wherein the memory device controller is configured to set a first signal threshold and a second signal threshold for each of the one or more integrated detectors;
transmit an indication, via the wireless communication device, to another device responsive to a determination that a value detected by the one or more integrated detectors is greater than or less than the first signal threshold; and
transmit an indication, via the wireless communication device, to another device responsive to a determination that a value detected by the one or more integrated detectors is greater than or less than the second signal threshold.

2. The system of claim 1, wherein:
the blood sample obtained from a person represents at least one of a whole blood, blood plasma, serum samples, isolated antibodies, blood compositions, or blood substitutes; and
the protein biosensor is configured to interact with at least one of a whole blood, blood plasma, serum samples, isolated antibodies, blood compositions, or blood substitutes, respectively.

3. The system of claim 1, wherein the second biological component within the protein biosensor represents lipids or microbial cells.

4. The system of claim 1, wherein:
the transducer is embedded into a metal alloy board that outputs the first and second signals; and
the metal alloy board is a silicon chip.

5. The system of claim 1, wherein:
the internal membrane within the protein biosensor is a semi-permeable multilayered membrane; and
at least one of the membrane layers represents graphene.

6. The system of claim 1, wherein:
the protein biosensor uses graphene coatings for binding to the blood sample; and
the graphene coating may be a layer of reduced graphene oxide.

7. The system of claim 1, wherein:
each of the one or more integrated detectors is further coupled to the wireless communication device via the detector output; and
the memory device transmit an indication to another device using the wireless communication device through the detector output.

8. The system of claim 1, wherein in response to the detected change within the blood sample, the memory device further updates, by the memory device controller, the first and second signal thresholds for each of the one or more integrated detectors.

9. The system of claim 8, wherein:
at least one of the one or more integrated detectors are configured to perform electrical, magnetic or optical measurements; and
the memory device is further configured to update, by the memory device controller, the first and second signal thresholds for each of the one or more integrated detectors based on the detected change in the electrical, magnetic or optical characteristics of the blood sample.

10. The system of claim 8, wherein:
at least one of the one or more integrated detectors contains a detector that measures temperature or relative molecular motion; and
the memory device is further configured to update, by the memory device controller, the first and second signal thresholds for each of the one or more integrated detectors based on the detected change in the characteristics of temperature or relative molecular motion of the blood sample.

11. The system of claim 1, wherein the indication identifies the presence of SARS-CoV-2 in said person when the first signal threshold or the second signal threshold for each of the one or more integrated detectors is set to a limit threshold for the SARS-CoV-2 virus strain.
